(19) United States
(12) Patent Application Publication
(10) Pub. No.: US 2016/0341694 A1
(43) Pub. Date: Nov. 24, 2016

(54) METHOD AND APPARATUS TO CONCENTRATE AND DETECT AN ANALYTE IN A SAMPLE

(71) Applicant: Headway Technologies, Inc., Milpitas, CA (US)

(72) Inventors: Jaime E. Arenas, San Ramon, CA (US); Celine Hu, Tiburon, CA (US); David Hu, Los Altos, CA (US); Greg Lyon, San Jose, CA (US); Bin Guo, Pleasanton, CA (US)

(21) Appl. No.: 15/154,701

(22) Filed: May 13, 2016

Related U.S. Application Data
(60) Provisional application No. 62/165,546, filed on May 22, 2015.

(51) Int. Cl. G01N 27/447; G01N 27/453; C12Q 1/68

(52) U.S. Cl. CPC G01N 27/44739; C12Q 1/6804; G01N 27/44791; G01N 27/453

(57) ABSTRACT

Devices and methods for detecting the presence or absence and/or one or more characteristics of analytes are disclosed. Embodiments of the disclosed devices include an actuator having a staging reservoir which can be operably connected with an electrophoresis assembly. In some embodiments, the disclosed methods include operating the subject devices, for example, by actuating the actuator to operably connect, e.g., fluidically connect, components of the devices, and electrophoresing or otherwise propelling a sample through portions of the devices. In some embodiments, the disclosed methods include detecting an analyte and/or analyzing properties of a sample, such as one or more characteristics of an analyte in a sample. The analyte of interest can be a charged molecule or can be modified to be charged using, for example, one or more ionic moieties.

### CROSS-REFERENCE

This application claims priority benefit of U.S. Provisional Application No. 61/165,546, filed May 22, 2015, which application is incorporated herein by reference in its entirety and for all purposes.

### FIELD OF THE INVENTION

The invention relates to sample preparation, microfluidics, macrofluidics, and detection of analytes present at low concentrations in a sample.

### INTRODUCTION

Analyte detection devices are particularly useful in a clinical laboratory setting, where such devices are regularly employed in clinical and other laboratory assays. However, such devices often require a high analyte concentration, a low sample volume, or both. For example, some analyte detection devices and/or systems employ an analyte amplification step, such as Polymerase Chain Reaction (PCR), to increase the analyte concentration in a set sample volume prior to detection. In addition, some analyte detection devices and/or systems, such as microfluidic devices and/or systems, are configured to work with relatively small sample volumes. Accordingly, there is a need in the art for devices and/or systems which increase analyte concentration and reduce sample volume thereby facilitating analyte detection by devices and/or systems which are limited by a minimum analyte concentration and/or a maximum sample volume. The methods, devices, and systems disclosed herein address these and related issues.

### BRIEF SUMMARY OF THE INVENTION

Devices and methods for detecting the presence or absence and/or one or more characteristics of analytes are disclosed. Embodiments of the disclosed devices include an actuator having a staging reservoir which can be operably connected with an electrophoresis assembly. In some embodiments, the disclosed methods include operating the subject devices, for example, by actuating the actuator to operably connect, e.g., fluidically connect, components of the devices, and electrophoresing or otherwise propelling a sample through portions of the devices. In some embodiments, the disclosed methods include detecting an analyte and/or analyzing properties of a sample, such as one or more characteristics of an analyte in a sample. The analyte of interest can be a charged molecule or can be modified to be charged using, for example, one or more ionic moieties.

More specifically, in some embodiments, the devices include an electrophoresis assembly including a sample reservoir; a second assembly, such as an analysis assembly, such as a microfluidic assembly, including an analysis element; and an actuator including a staging reservoir and actuable between a first configuration and a second configuration. In some embodiments, a staging reservoir is operably, e.g., fluidically, connected with the sample reservoir when the actuator is in the first configuration and is operably, e.g., fluidically, connected with the analysis area when the actuator is in the second configuration. In some embodiments, an actuator includes a valve and actuating the actuator includes rotating the valve, such as rotating the valve 90° or other suitable distance, between a first configuration and a second configuration. In some embodiments, actuating the actuator includes sliding the actuator or a portion thereof from a first position to a second position and thereby moving the actuator or portion thereof between a first configuration and a second configuration.

In some embodiments, the subject devices include an electrophoresis assembly which is configured to move an analyte via electrophoresis, for example, from the sample reservoir to the staging reservoir. In some embodiments, the electrophoresis assemblies include a first electrode, e.g., a microelectrode, and a second electrode, such as a microelectrode, wherein the electrodes are configured so that an electric field may be generated between the electrodes. A variety of suitable electrodes are known in the art, including microelectrodes (e.g., electrodes having at least one dimension of from 0.1 to 50 μm). A suitable electrode may have any suitable electrode area, such as from 10⁻¹⁴ m² to 10⁻² m², or greater. It should be noted that any suitable embodiment described herein the term microelectrode may be used in place of the term electrode and vice versa. In some aspects, generating an electric field between the electrodes moves an analyte via electrophoresis from the sample reservoir to the staging reservoir.

Embodiments of the subject disclosure also include devices having an electrophoresis assembly including a first buffer reservoir, such as a low salt buffer reservoir and a second buffer reservoir, such as a high salt buffer reservoir operably, e.g., fluidically, connected to the sample reservoir. In some embodiments, the first buffer reservoir and the sample reservoir are one and the same, i.e., a single reservoir which includes or is configured to include both sample and buffer. In other embodiments, the first buffer reservoir may be a separate reservoir which is fluidically connected to the sample reservoir. As used herein, “low salt buffers” are buffers having a relatively low conductivity, e.g., a lower conductivity than a high salt buffer. Low salt buffers may be solutions containing a 0.01-300 mM, inclusive, such as 0.1-300 mM, inclusive, such as 1-99 mM, inclusive, concentration of charged ions. Low salt buffers may also be solutions containing a 0.1 mM to 1M, such as 1 mM to 0.3 M, such as 10 mM to 0.1 M, inclusive, concentration of charged ions. Also, as used herein, “high salt buffers” are buffers having a relatively high conductivity, e.g., a higher conductivity than a low salt buffer. High salt buffers may be solutions containing 100-3000 mM, inclusive, such as 100-2000 mM, inclusive, such as 500-1000 mM, inclusive, ion concentration. In some embodiments, high salt buffers contain greater than 300 mM concentration of charged ions, such as greater than 500 mM, greater than 1M, or greater than 2 M. High salt buffers may also be solutions containing a 0.1 M to 5 M, such as 0.15 M to 2 M, such as 0.2 M to 1 M, inclusive, ion concentration. Ions may include monovalent ions such as Na⁺, K⁺, Li⁺, H⁺, OFF; multivalent ions such as Mg⁺⁺, Ca⁺⁺, Mn⁺⁺, borate, tetraborate, phosphate, sulfate, bicarbonate; or charged species of organic salts and buffers such as tris, tricine, MOPS, CAPS, MES, taurine, urea, citrate, acetate, glycine, etc. Other species affecting the conductivity of a buffer solution may include surfactants, detergents, ampholites, zwitterions, etc. In some aspects, the low salt buffer reservoir is operably, e.g., fluidically, connected to the high salt buffer reservoir when the actuator is in a first configuration and/or is not operably, e.g., fluidically, connected to the high salt buffer reservoir when the actuator is in the second configuration.

In some embodiments of the subject devices, the electrophoresis assembly includes a membrane configured, such as by having a characteristic porosity, to inhibit movement of analyte through the membrane. Such a membrane may be positioned within the device downstream of the staging reservoir and configured to concentrate the analyte in and/or near the staging reservoir. Filtering elements, such as membranes according to the subject embodiments may include one or a combination of materials including polymeric materials, such as plastic and/or rubber, and/or paper materials, and/or gels, such as hydroxymethylcellulose, and are described in greater detail below. In some embodiments, a staging reservoir is operably connected to a large volume reservoir via a connector, such as a conduit, such as a tube.

Embodiments of the devices may include a housing, and in some embodiments, at least a portion of a sample reservoir is defined by the housing. In some embodiments, at least a portion of the actuator, for example, a portion of the actuator including a staging reservoir, is positioned within a housing of the device.

Embodiments of the devices also include an analysis assembly, such as a microfluidic assembly including a propulsion assembly, such as one or more pumps and associated connectors, configured to propel the analyte through a portion of the analysis assembly, such as the microfluidic assembly, for example, from the staging reservoir to the analysis element. In some embodiments, an analysis assembly, such as a microfluidic assembly includes a first microelectrode and a second microelectrode, wherein the microelectrodes are configured such that an electric field may be generated between the microelectrodes, and wherein generating an electric field between the microelectrodes moves an analyte via electrophoresis from the staging reservoir to the analysis element.

In some embodiments, a solution sample in which an analyte has not yet been concentrated, such as concentrated within a staging reservoir using electrophoresis as described herein, may be referred to as a sample, an initial sample, and/or an unprocessed sample. A solution sample in which an analyte has been concentrated, such as concentrated within a staging reservoir using electrophoresis as described herein, may be referred to as a sample and/or a processed sample. Furthermore, according to the subject embodiments, “processing” a sample refers to concentrating an analyte in and/or near the staging reservoir. Accordingly, processing a sample converts an unprocessed sample into a processed sample, such as a sample having a concentrated analyte therein and/or a smaller volume than the unprocessed sample. Processing a sample may include conducting electrophoresis using an electrophoresis assembly, as described herein.

In various embodiments, a sample, e.g., an unprocessed sample, and/or a sample reservoir, such as a large volume reservoir, has a volume of, for example, 0.1 μL or greater, such as 1 μL or greater, such as 5 μL or greater, such as 10 μL or greater, such as 100 μL or greater, such as 1 mL or greater, such as 100 mL or greater, such as 1000 mL or greater, or 5000 mL or greater. In some embodiments, a sample, e.g., an unprocessed sample, and/or a sample reservoir, such as a large volume reservoir, has a volume of 100 μL or less, such as 10 μL or less, such as 1 μL or less. In some embodiments, a sample, e.g., an unprocessed sample, and/or a sample reservoir, such as a large volume reservoir, has a volume ranging, for example, from 0.01 μL to 5000 mL, 0.1 μL to 1000 mL, 1 μL to 50 mL, 1 μL to 1 mL, 1 μL to 500 μL, 1 μL to 100 μL, or 5 μL to 50 μL, with each of the listed ranges provided in this disclosure being inclusive, i.e., including, and not excluding, both of their terminal values. In various embodiments, a sample reservoir, such as a large volume reservoir, is configured to receive entirely therein a sample, e.g., an unprocessed sample. As such, in some aspects, a sample reservoir, such as a large volume reservoir, has a volume equal to or larger, such as slightly larger, than a volume of a sample, e.g., an unprocessed sample, received therein.

In various aspects, a staging reservoir, as described herein, is configured to receive entirely therein a sample, such as a processed sample. As such, in some aspects, a staging reservoir has a volume equal to or larger, such as slightly larger, than a volume of a sample, e.g., a processed sample, received therein. In some embodiments, a sample, e.g., a processed sample, and/or a staging reservoir, has a volume ranging, for example, from 0.01 μL to 100 mL, 0.01 μL to 1 mL, 0.01 μL to 1 mL, 0.1 μL to 500 μL, 0.1 μL to 100 μL, 1 μL to 50 μL, or 1 μL to 10 μL, each inclusive. Also, in various embodiments, a sample, e.g., a processed sample, and/or a staging reservoir, has a volume, for example, of 0.01 μL or greater, 0.1 μL or greater, such as 1 μL or greater, such as 5 μL or greater, such as 10 μL or greater, such as 50 μL or greater, such as 100 μL or greater, such as 500 μL or greater, such as 1 mL or greater, such as 10 mL or greater, such as 100 mL or greater. In some embodiments, a sample, e.g., a processed sample, and/or a staging reservoir, has a volume, for example, of 0.01 μL or less, 0.1 μL or less, 1 μL or less, 5 μL or less, 10 μL or less, 50 μL or less, 100 μL or less, 500 μL or less, 1 mL or less, 10 mL or less, or 100 mL or less.

In some embodiments, a sample, e.g., an unprocessed sample or a processed sample, and/or a reservoir, such as a sample reservoir, such as a large volume reservoir, or a staging reservoir, may have a volume in an inclusive range from 10 μl to 50 mL, such as from 20 μl to 20 mL, such as from 20 μl to 100 μl. A sample, e.g., an unprocessed sample or a processed sample, and/or a reservoir, such as a sample reservoir, such as a large volume reservoir, or a staging reservoir, may have a volume in an inclusive range from 100 μl to 200 μl, from 200 μl to 300 μl, from 300 μl to 400 μl, from 400 μl to 500 μl, from 500 μl to 600 μl, from 600 μl to 700 μl, from 700 μl to 800 μl, from 800 μl to 900 μl, from 900 μl to 1 mL, from 1 mL to 10 mL, from 10 mL to 25 mL, or from 25 mL to 50 mL. In some embodiments, a sample, e.g., an unprocessed sample or a processed sample, and/or a reservoir, such as a sample reservoir, such as a large volume reservoir, or a staging reservoir, has a volume of from 10 μl to 500 μl, or from 500 μl to 10 mL. In some embodiments, the sample, e.g., an unprocessed sample or a processed sample, and/or a reservoir, such as a sample reservoir, such as a large volume reservoir, or a staging reservoir, has a volume of 1 μl or greater, such as 10 μl or greater, such as 20 μl or greater, such as 50 μl or greater, such as 100 μl or greater, such as 500 μl or greater, such as 1 mL or greater.

In some embodiments of the disclosed embodiments, an unprocessed sample, and/or a sample reservoir, such as a large volume reservoir, has a volume which is larger, such as significantly larger, than a volume of a processed sample, and/or a staging reservoir. For example, in some aspects, a volume of an unprocessed sample, and/or a sample reservoir, such as a large volume reservoir, has a volume which is, for example, 2× (two times) or more, 3× or more, 5× or more, 10× or more, 20× or more, 50× or more, 100× or more, 500× or more, 1000× or more, 2000× or more, 5000× or more, or 10000× or more, than the volume of a processed sample, and/or a staging reservoir. In some embodiments, a volume of a processed sample, and/or a staging reservoir is smaller by (e.g., reduced by) a factor of, for example, 2× or more, 3× or more, 5× or more, 10× or more, 20× or more, 50× or more, 100× or more, 500× or more, 1000× or more, 2000× or more, 5000× or more, or 10000× or more, relative to a volume of an unprocessed sample, and/or a sample reservoir, such as a large volume reservoir. In some embodiments, a volume of a processed sample, and/or a staging reservoir is smaller than a volume of an unprocessed sample, and/or a sample reservoir, such as a large volume reservoir by a factor ranging, for example, from 1.5× to 5000×, 1.5× to 1000×, 2× to 5000×, 2× to 1000×, 2× to 500×, 2× to 100×, 2× to 50×, 2× to 10×, or 2× to 5×, each inclusive.

The present disclosure also includes methods for introducing an analyte in a sample to an analysis assembly, such as a microfluidic assembly or a portion thereof. In some embodiments of the present disclosure, the methods are methods of introducing an analyte in a sample to an analysis assembly, such as a microfluidic assembly. Such methods may include electrophoresing an analyte, e.g., by applying an electric potential to a sample including the analyte, into a staging reservoir of an actuator. Applying such an electric potential may include generating an electric field between two electrodes. Electrophoresis according to the disclosed embodiments may include the use of an electrophoresis assembly, e.g., as described herein to move the analyte within the device. The analyte can be moved within the device, for example, by electrophoresing the analyte from the sample reservoir, e.g., large volume reservoir, to the staging reservoir. In some embodiments, the sample is moved from the sample reservoir, e.g., large volume reservoir, to the staging reservoir via a connector, such as a conduit, such as a tube. In some embodiments, the staging reservoir has one or more characteristics of the large volume reservoirs described herein.

Methods according to the present disclosure may also include concentrating the analyte in the staging reservoir. Concentrating an analyte may be achieved by applying an electric field across a membrane or other filtering element which restricts flow of analyte therethough. Such an electric field may be generated between electrodes, e.g., microelectrodes on respective sides of the membrane.

In some embodiments, the actuator can be moved, e.g., rotated, to operably, e.g., fluidically, connect the staging reservoir to an analysis element of the analysis assembly, such as the microfluidic assembly. In some embodiments, the actuator includes a valve and actuating the actuator includes rotating the valve, e.g., rotating the valve 90° or other suitable distance, from a first configuration to second configuration. In some embodiments, actuating the actuator includes sliding the actuator or a portion thereof from a first position to a second position and thereby moving the actuator or portion thereof between a first configuration and a second configuration.

In various embodiments, the disclosed methods include a step of moving, e.g., propelling, the sample or an analyte contained therein into the analysis element of the analysis assembly, such as the microfluidic assembly, and/or performing analysis on the sample or an analyte contained therein. Such propelling may be achieved by applying an electric field between electrodes such as, for example, a first microelectrode at a first end of the analysis assembly, such as the microfluidic assembly and a second microelectrode at a second end of the assembly. Propelling may also be achieved by applying a pressure, such as a fluidic pressure, to a sample using a propulsion element such as a pump. It should be noted that either a positive or a negative pressure pump may be utilized and positioned appropriately to accomplish the desired movement.

In some embodiments, the disclosure provides methods for introducing or applying an analyte of interest in a sample to a portion of an analysis assembly, e.g., a microfluidic assembly, such as an analysis element. Such an introduction can be made by providing one or more aqueous samples in one or more large volume reservoirs of an electrophoresis assembly, wherein the samples contain one or more analytes of interest. In some embodiments, e.g., where electrophoresis is used to move an analyte, the analytes of interest are charged or associated with a charged molecule, such as an ionic molecule or a carrier molecule that is charged. Such analytes can then be electrophoresed, such as electrophoresed from or via the large volume reservoir to a staging reservoir and concentrated therein. The concentrated analyte-containing sample or an analyte contained therein is then moved to an analysis element and analysis conducted thereon. The concentrated analyte-containing sample or analyte contained therein may be moved to an analysis element by conducting electrophoresis, utilizing a pressure induced flow, and/or via mechanical transfer. In some embodiments, a concentrated analyte-containing sample or analyte contained therein may be transferred, such as transferred indirectly, to an analysis element, such as an analysis element of a separate device manually or using one or more robotic components and/or semi-automated methods, such as methods of which, at least a part, or only a part, involve a computer-controlled transfer. The large volume reservoir can include, for example, a container such as a microwell plate, an eppendorf tube, or a test tube. The large volume reservoir can be a well that is integral with a portion of the device, such as a housing. In some embodiments, the analyte of interest is a biomolecule, such as a peptide, a protein, a nucleic acid, a lipid or a sugar. An analyte associated with a charged molecule can be, for example, an analyte attached to an ionic moiety or an analyte attached to an antibody that is ionically charged.

In some embodiments of the present disclosure, a sample, e.g., an unprocessed sample, is first admixed with an antibody which specifically binds to the analyte of interest. Such an antibody can, in some embodiments, and either before or after binding to the analyte, be modified with at least one ionic moiety. Alternatively, the analyte of interest can be modified to be charged, for example, directly with an ionic moiety. In some embodiments, one or more polynucleotides can be used to modify antibodies specific for one or more analytes, e.g., via a covalent linkage, to introduce a charge thereto. In addition, or alternatively, the analytes themselves can be modified with one or more polynucleotides to introduce a charge thereto.

In some embodiments of the present disclosure, the analysis element is a capture site and the method involves moving the charged molecule(s) across the capture site. In some embodiments, the capture site includes at least one capture agent which allows the analyte of interest to be captured. The movement over the capture site may be by pressure induced flow, electrophoresis, or any other suitable method. The method can further include detecting the analyte bound by the capture agent. The capture agent can bind the analyte or an ionic moiety bound to the analyte. The capture agent may be any suitable capture moiety known in the art, e.g., an antibody or a nucleic acid. The detection can involve detecting a label on the antibody, the analyte, or the ionic moiety.

Various methods of attaching ionic moieties to analytes of interest and subsequently detecting such analytes, e.g., via the attachment of antibody intermediates, are described in U.S. Pat. No. 8,263,022, the disclosure of which is incorporated by reference herein in its entirety and for all purposes. Such methods may be utilized in connection with any of the embodiments described herein as appropriate.

In some embodiments of the present disclosure, the analyte of interest is attached to a particle, e.g., a microparticle, and/or other suitable solid support before electrophoresis. The microparticle can be a magnetic microparticle. The microparticle or other suitable solid support may be coated with a capture agent having a specific affinity for the analyte, e.g., a nucleic acid or antibody capable of specifically binding to the analyte. In some embodiments, the microparticle is coated with one or more capture agents including at least one receptor, antibody, or anti-ligand specific for the analyte of interest. The method may include the step of removing the analyte from the microparticle prior to electrophoresis. Where the microparticle is coated with an antibody, the antibody can, in some embodiments, recognize the analyte or be the analyte. As described in more detail below, the term “antibody” as used herein refers to immunoglobulins including two heavy and two light chains, and antigen binding fragments thereof (including, e.g., Fab, Fab′ F(ab′)2, Fabc, and scFv). The term “antibody” also includes one or more immunoglobulin chains or fragments that may be chemically conjugated to, or expressed as, fusion proteins with other proteins, single chain antibodies, and bispecific antibodies. Antibodies used in association with the disclosed devices and methods can be monoclonal or polyclonal. A second antibody can also be provided and can bind to a different site on the analyte. The detection of the analyte can involve detecting a label on the second antibody. For example, a first and/or second antibody can be labeled with one or more fluorophores and analyzed with an apparatus by exciting the fluorophores with a laser of appropriate wavelength and measuring the emitted fluorescent light. In some embodiments, an ionic moiety can be attached at any step before electrophoresis via an indirect attachment. Such an indirect attachment can be, for example, via an avidin/biotin attachment. The detection of an analyte can also be indirect. For example, according to the subject embodiments, the presence of an analyte in a sample may be determined by detecting the presence of a product, such as an enzymatic product, of an enzyme, that is associated with the analyte.

### BRIEF DESCRIPTION OF THE DRAWINGS

FIG. 1 provides an embodiment of a system including an electrophoresis assembly and a microfluidic assembly according to the present disclosure.

FIG. 2 provides a partial cut-away view of one embodiment of a portion of a device according to the present disclosure.

FIG. 3 provides a partial cut-away view of one embodiment of a portion of a device according to the present disclosure.

FIGS. 4, A and B, provide schematic representations of an embodiment of the subject device. FIG. 4, A, depicts a device having an actuator in a first configuration whereas FIG. 4, B, depicts the device with the actuator in a second configuration.

FIG. 5 provides a graphical representation showing a reduction in the Limit of Detection (LOD) of an oligonucleotide analyte using a device and method according to the present disclosure.

FIG. 6 provides an embodiment of a system including an electrophoresis assembly according to the present disclosure.

FIGS. 7, A and B, provide partial cut-away views of an embodiment of a system including an electrophoresis assembly and a sliding actuator according to the present disclosure. FIG. 7A shows the actuator in a first configuration and FIG. 7B shows the actuator in a second configuration.

FIGS. 8, A and B, provide partial cut-away views of an embodiment of a system including an electrophoresis assembly and a rotating actuator according to the present disclosure. FIG. 8A shows the actuator in a first configuration and FIG. 8B shows the actuator in a second configuration.

FIGS. 9, A and B, provide partial cut-away views of one embodiment of a portion of a device according to the present disclosure. FIG. 9A shows a portion of a device in a first configuration and FIG. 9B shows the portion of the device in a second configuration.

FIG. 10 provides an embodiment of a system including an electrophoresis assembly according to the present disclosure.

FIG. 11 provides an embodiment of a system including an electrophoresis assembly according to the present disclosure.

### DETAILED DESCRIPTION OF THE INVENTION

### I. Introduction

Devices and methods for detecting the presence or absence or one or more characteristics of analytes are disclosed. Embodiments of the disclosed devices include an actuator having a staging reservoir which can be operably connected with an electrophoresis assembly. In some embodiments, the disclosed methods include operating the subject devices, for example, by actuating the actuator to operably connect, e.g., fluidically connect, components of the devices, and electrophoresing or otherwise propelling a sample through portions of the devices. In some embodiments, the disclosed methods include a step of detecting the presence or absence of one or more analytes and/or analyzing properties of a sample, such as one or more characteristics of an analyte in a sample. The analyte of interest can be a charged molecule or can be modified to be charged using, for example, one or more ionic moieties.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “an analyte” includes a plurality of such analytes and reference to “the material” includes reference to one or more materials and equivalents thereof.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent such publications may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

A. Devices

As noted above, the present disclosure includes devices for detecting the presence or absence or one or more characteristics of analytes. Embodiments of the disclosed devices include one or more electrophoresis assemblies, such as an electrophoresis assembly including a sample reservoir. As used herein, the term “electrophoresis” refers to the movement of charged molecules or particles in solution in response to an electric field. As such, an “electrophoresis assembly” is a device or portion thereof configured for moving one or more charged molecules or particles in a medium, e.g., a solution, in response to an electric field. In various embodiments, the disclosed electrophoresis assemblies include one or more sample reservoirs. A sample reservoir is a container, e.g., a container within an electrophoresis assembly, sized and/or shaped to receive a sample, as described herein. A sample reservoir may have one, two, or more, open ends and may include, for example, an opening for receiving fluid at a first end and an opening for expelling fluid at a second end. A sample reservoir may have, for example, a tubular or conical shape extending from a first opening at a first end to a second opening at a second end opposite the first end. In some embodiments, a sample reservoir is a large volume reservoir. Furthermore, electrophoresis assemblies may be microfluidic devices or have one or more of the characteristics of microfluidic devices described herein. In some embodiments, electrophoresis assemblies are not microfluidic devices, such as microfluidic assemblies. In some aspects, electrophoresis assemblies may be macrofluidic devices, such as macrofluidic assemblies.

Various embodiments of the disclosed devices also include one or more analysis assemblies. The phrase “analysis assembly”, as used herein, refers to an assembly having one or more components for analyzing a sample or an analyte contained therein, such as analyzing a sample by detecting an analyte and/or characteristics thereof, as described herein. For example, an analysis assembly may include an analysis element. One type of analysis assembly is a microfluidic assembly, such as a microfluidic assembly as described herein. Accordingly, any of the characteristics of microfluidic assemblies described herein may also alternatively, or additionally, be characteristics of analysis assemblies. Also, in some embodiments, an analysis assembly is not a microfluidic assembly. In other words, in some embodiments, analysis assemblies do not include one or more aspects of microfluidic devices, such as microfluidic components.

As noted above, embodiments of the disclosed devices may also include one or more analysis assemblies, such as microfluidic assemblies, such as a microfluidic assembly including one or more analysis elements. A “microfluidic assembly”, as used herein, is an assembly including one or more aspects of microfluidic devices. A microfluidic device may have, for example, one or more channels or vessels with a size less than 1 mm in at least one dimension. The device may have at least one channel or vessel with at least one dimension, e.g., diameter and/or radius and/or length and/or width and/or height, of 1.0 mm or less, 0.5 mm or less, 0.2 mm or less, 0.1 mm or less, 50 μm or less, 20 μm or less, 10 μm or less, 5 μm or less, or 1 μm or less. In some embodiments, the device may have at least one channel or vessel with at least one dimension, e.g., diameter and/or radius and/or length and/or width and/or height, of about 1.0 mm to about 0.5 mm, about 0.5 mm to about 0.2 mm, about 0.2 mm to about 0.1 mm, about 0.1 mm to about 50 μm, about 50 μm to about 20 μm, about 20 μm to about 10 μm, about 10 μm to about 5 μm, or about 5 μm to about 1 μm. Furthermore, microfluidic(s) devices may be configured to receive and/or channel one or more of the solutions described herein therethrough. An analysis element, area or zone, or a detection element, area or zone (which terms may be used interchangeably herein) of an analysis assembly, such as a microfluidic assembly, is an element, area or zone, which may be integral with and/or included in an analysis assembly or separate, e.g., reversibly disconnectable, from an analysis assembly, and which is configured for detecting the presence or absence of an analyte and/or one or more properties of an analyte. Furthermore, in some embodiments, the subject devices only include an electrophoresis assembly and/or do not include an analysis assembly, such as a microfluidic assembly, as described herein. Such devices may also include an analysis element.

As noted above, various embodiments of the subject devices include one or more analysis elements for detecting an analyte and/or analyzing one or more characteristics thereof. Such an analysis element may be included in an analysis assembly, such as a microfluidic assembly or may be in a detection device which is separate and independent from an analysis assembly, such as a microfluidic assembly, and/or an electrophoresis assembly as described herein. In devices wherein an analysis element is included in a separate and independent, e.g., unattached, device from an electrophoresis assembly, the devices may include one or more transfer elements for transferring a sample to the analysis element. Accordingly, the subject methods may include transferring, e.g., transferring via a container such as a tube or pipet, a sample from an electrophoresis assembly or a portion thereof, such as a staging reservoir, to an analysis element.

Microfluidic devices or assemblies as described herein can include one or more micropumps, valves, temperature regulators, etc. In some embodiments, microfluidic devices or assemblies are not included in electrophoresis assemblies. In some embodiments, microfluidic devices or microfluidic assemblies do not include electrodes such that a substance can be propelled through the device by generating an electric field between the electrodes. In other words, in some embodiments of microfluidic devices or assemblies, a substance, such as a fluid, may be propelled therethrough only by a means other than by electrophoresis, such as by a positive or negative pressure pumping element.

Devices according to the present disclosure may also include one or more actuators, such as an actuator having a staging reservoir. A staging reservoir is a container or space, e.g., a container or space within an actuator, sized and/or shaped to receive a sample, as described herein. A staging reservoir may have one, two, or more, open ends and may include, for example, an opening for receiving fluid at a first end and an opening for expelling fluid at a second end. A staging reservoir may have, for example, a tubular shape extending from a first opening at a first end to a second opening at a second end opposite the first end. Staging reservoirs according to the subject embodiments may also be movable, e.g., rotatable or slidable, e.g., manually rotatable and/or automatically rotatable (or manually slidable and/or automatically slidable), within an actuator and/or within a housing.

Embodiments of actuators may be actuable, e.g., rotatable or slidable, between two or more configurations, such as between three, four, or five, or more configurations. For example, an actuator may be actuable between a first and second configuration, wherein the staging reservoir is operably connected, e.g., fluidically connected, with an electrophoresis assembly or a portion thereof, such as a sample reservoir, when the actuator is in the first configuration and/or is operably connected, e.g., fluidically connected, with an analysis assembly, such as a microfluidic assembly or a portion thereof, and/or an analysis element, when the actuator is in the second configuration. As described further below, in some embodiments, actuating the actuator may include rotating the actuator or a portion thereof, e.g., an end portion, 360° or less, such as 180° or less, such as 90° or less, such as 45° or less, such as 30° or less, such as 15° or less in a single rotational direction and/or in two opposite rotational directions, e.g., rotating an amount in a first direction and then an equal amount in a second direction opposite the first. In some embodiments, actuating the actuator as described herein may include rotating the actuator or a portion thereof, e.g., an end portion, in a radius extending from 0° to 360°, inclusive, such as from 0° to 180°, such as from 0° to 90°, such as from 0° to 45°, such as from 0° to 30°, such as from 0° to 15°, each inclusive, in a first rotational direction and/or in a second rotational direction opposite the first. As described further below, in some embodiments, actuating the actuator may include sliding the actuator or a portion thereof, e.g., slidably moving the actuator from a first configuration to a second configuration, e.g., in a horizontal, vertical or other suitable direction. Such slidable movement may be relative to a housing and/or a portion of an electrophoretic assembly. Also, in various embodiments, the subject devices include one or more actuators, such as only a single actuator, or two, three, four, five, ten, fifty, one-hundred, or one-thousand, or more actuators.

By “operably connected”, as used herein, is meant connected in a specific way, e.g., in a manner allowing fluid to move (i.e., fluidically) and/or electricity to be transmitted, that allows the disclosed devices and their various components to operate effectively in the manner described herein. For example, a staging reservoir operably connected with a sample reservoir may allow a sample, such as a fluid sample, to be transmitted, e.g., flowed, between the staging reservoir and the sample reservoir such that the sample can be transferred from the sample reservoir to the staging reservoir. Likewise, for example, a staging reservoir operably connected with an analysis element may allow a sample, such as a fluid sample, to be transmitted, e.g., flowed, between the staging reservoir and the analysis element such that the sample can be transferred from the staging reservoir to the analysis element. In addition, an operable connection may include, for example, a fluidic connection and/or an electrical connection.

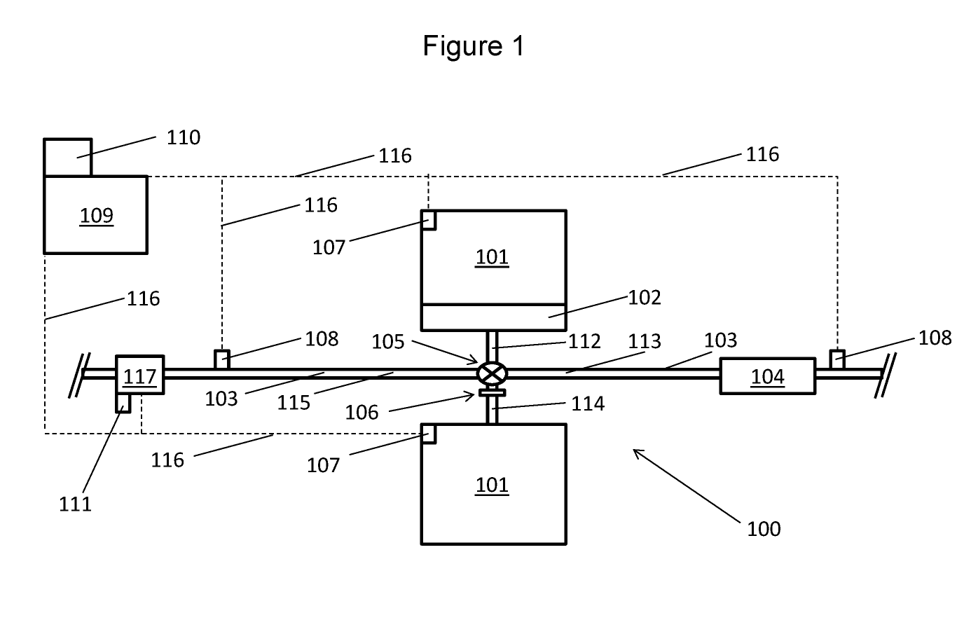

A schematic of an embodiment of a device according to the present disclosure is shown in FIG. 1. FIG. 1 specifically illustrates an electrophoresis assembly 101 having a sample reservoir 102, an analysis assembly, such as a microfluidic assembly 103 and an analysis element 104. The device shown in FIG. 1 also includes an actuator 105 having a staging reservoir and which is actuable between a first configuration and a second configuration, wherein the staging reservoir is operably, e.g., fluidically, connected with the sample reservoir 102 when the actuator is in the first configuration and is operably connected with the analysis element 104, via the microfluidic assembly 103, when the actuator 105 is in the second configuration.

As is also shown in FIG. 1, the disclosed devices may include an electrophoresis assembly 101 including one or more filtering elements 106, such as a membrane, positioned downstream of an actuator 105. The electrophoresis assembly 101 of the device 100 shown in FIG. 1 also includes electrodes, e.g., microelectrodes 107, operably coupled, such as electrically coupled, to one or more power supply 109, such as a power supply 109 having a power supply controller 110, such as an electronic controller including, for example, a computer, for turning the power supply 109 on and off and controlling the voltage and/or current provided by the power supply 109. Operable connections 116, e.g., electrical connections, each one of which are optional, are shown in FIG. 1. Other operable connections which are not shown are possible as well. A first microelectrode and a second microelectrode of an electrophoresis assembly may be configured so that an electric field may be generated between the electrodes to move an analyte, for example, from a sample reservoir to a staging reservoir. The one or more power supply 109 may also be operably coupled to optionally present electrodes, e.g., microelectrodes 108, within the microfluidic assembly 103 of the device 100. The microelectrodes 108 of the microfluidic assembly may be operatively coupled to the same or a different power supply than the microelectrodes 107 of the electrophoresis assembly 101. A first microelectrode and a second microelectrode of a microfluidic assembly may be configured so that an electric field may be generated between the electrodes to move an analyte, for example, from a staging reservoir to an analysis element.

Furthermore, a microfluidic assembly 103, such as a microfluidic assembly that does not include one or more electrodes, also may optionally include a propulsion assembly including a propulsion element 117, such as a pump, such as a microfluidic pump. The propulsion element 117 also may include a propulsion controller 111, including for example, a computer and/or manual controller, and configured for controlling the flow of one or more liquids within the microfluidic assembly by adjusting the amount of propulsion provided by the propulsion element 117. In some embodiments, this may include an adjustment of the flow rate of a fluid within microfluidic assembly 103. In other words, the propulsion assembly may be configured to propel an analyte within a device, for example, from a staging reservoir to an analysis element.

As is described in greater detail below, the embodiment of the device shown in FIG. 1 may be configured such that an analyte, including one or more particles associated with, e.g., bound to, an analyte, may be electrophoresed from sample reservoir 102 into the actuator 105 or a portion thereof, e.g., a staging reservoir, using the electrophoresis assembly 101. The actuator 105 is configured to be actuated, e.g., manually or automatically actuated, such as actuated by rotation, such as actuated by rotation within a vertical plane, e.g., a plane perpendicular to a ground surface, or a horizontal plane, to disconnect the staging reservoir from the electrophoresis assembly 101 or a portion thereof, such as a sample reservoir, and/or operably connect the staging reservoir with a microfluidic assembly 103 and/or an analysis element 104. In some embodiments, actuation includes rotating an actuator or a portion thereof along an arc length which defines a vertical or horizontal plane. In various aspects, actuation includes moving, e.g., rotating, an actuator between a first and second configuration and may also include moving an actuator between one or more additional configurations, such as a third, fourth, and/or fifth configuration. In various aspects, actuation includes moving, e.g., rotating, an actuator between five or more, such as ten or more, such as twenty or more, such as fifty or more configurations. The microfluidic assembly 103 is configured to thereafter move the analyte out of the staging reservoir of the actuator 105 to an analysis element 104.

In addition, and as is described further below, the elements shown in FIG. 1, e.g., sample reservoir and/or connector 112, etc., may be filled with fluid, such as one or more suitable buffers that serve the purpose of connecting the electrodes 107 and/or 108 to form an electrical field. Forming such an electric field may allow a charged analyte to be electrophoresed, for example, from a sample reservoir 102 to a staging reservoir of actuator 105. Elements which are illustrated in FIG. 1 and their corresponding numerical designations are summarized as follows:

TABLE 1

| Element | Numerical Designation |
|---|---|
| Electrophoresis assembly | 101 |
| Sample reservoir | 102 |
| Microfluidic assembly | 103 |
| Analysis element | 104 |
| Actuator | 105 |
| Filtering element(s) | 106 |
| Electrodes | 107, 108 |
| Power supply | 109 |
| Power supply controller | 110 |
| Propulsion controller | 111 |
| Connectors | 112, 113, 114, 115, |
| Operably connection(s) | 116 |
| Propulsion element | 117 |

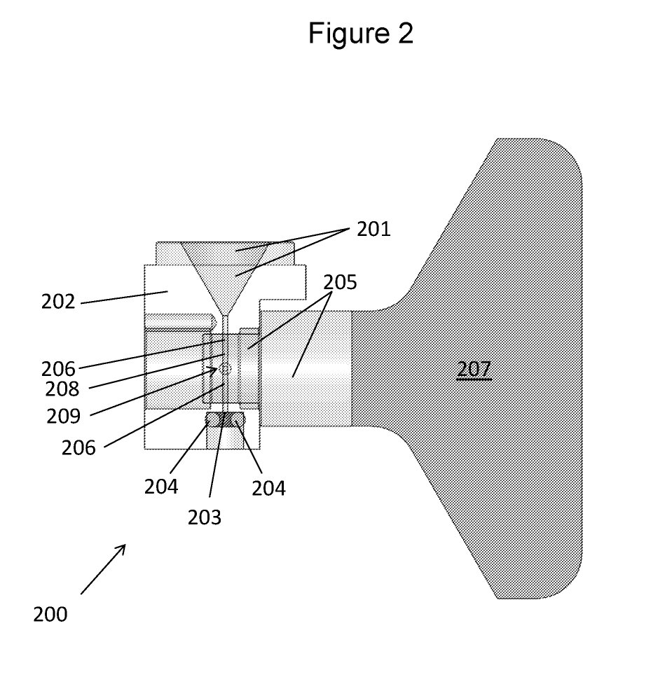

One embodiment of a portion of a disclosed device is shown in FIG. 2. FIG. 2 specifically provides a partial cut-away version of a portion 200 of an exemplary device and shows a sample reservoir 201 of an electrophoresis assembly. The device 200 also includes a housing 202 defining the sample reservoir 201. The device shown in FIG. 2 also includes a filtering element, such as a filter, for example, at or proximate position 203, and held within the housing 202 by a retaining element, e.g., an O-ring 204. The portion of the device shown in FIG. 2 also includes an actuator 205 having a staging reservoir 206 and a handle 207 for manual actuation, e.g., rotation, of the actuator 205. The staging reservoir 206 can be actuated between a first configuration 208 and a second configuration 209 when the actuator 205, which is shown in the first configuration, is actuated. Elements which are illustrated in FIG. 2 and their corresponding numerical designations are summarized as follows:

TABLE 2

| Element | Numerical Designation |
|---|---|
| Sample reservoir | 201 |
| Housing | 202 |
| Proximate position of filter | 203 |
| O-ring | 204 |
| Actuator | 205 |
| Staging reservoir | 206 |
| Handle | 207 |
| First configuration (of staging reservoir) | 208 |
| Second configuration (of staging reservoir) | 209 |

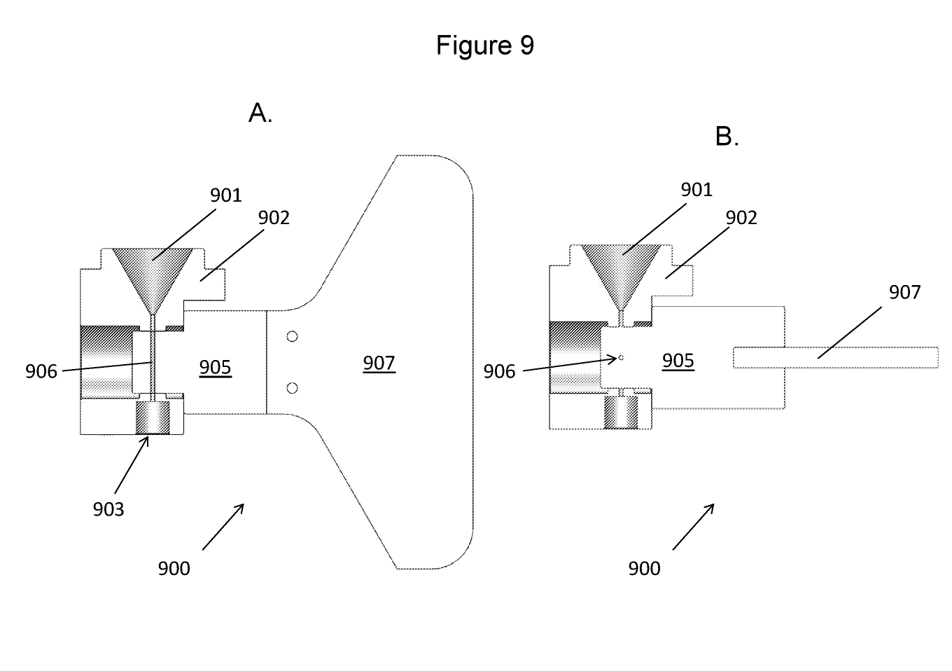

A simplified cross-sectional view of a device as provided in FIG. 2 is shown in FIGS. 9A and 9B. The embodiment of the device of FIGS. 9A and 9B may have any of the characteristics or features of the embodiment of the device shown in FIG. 2. FIGS. 9A and 9B show a device including a sample reservoir 901 of an electrophoresis assembly. The device 900 also includes a housing 902 defining the sample reservoir 901. The device shown in FIGS. 9A and 9B also includes a filtering element, such as a filter, for example, at or proximate position 903, and held within the housing 902 by a retaining element. The portion of the device shown in FIGS. 9A and 9B also includes an actuator 905 having a staging reservoir 906 and a handle 907 for manual actuation, e.g., rotation, of the actuator 905. The actuator 905 and portions thereof, e.g., the staging reservoir 906 and/or the handle 907, can be actuated, such as actuated by rotation, between a first configuration as shown in FIG. 9A and a second configuration as shown in FIG. 9B, when the actuator 905, is actuated. As is shown in FIG. 9A, when the actuator 905 is in the first configuration, the staging reservoir 906 is operably coupled to the sample reservoir 901. Actuating the actuator 905 to the second configuration causes the staging reservoir 906 to no longer be operably connected to the sample reservoir 901. Instead, the staging reservoir 906 becomes operably connected to another aspect, such as an analysis assembly, such as a microfluidic assembly and/or a sample transfer element and/or a detection device and/or an analysis element, as each of such components are described herein. Actuation of the actuator 905 can be manual or automatic. Actuation may also include rotating the actuator and/or portions thereof about an axis, such as a horizontal and/or vertical axis, such as an axis of symmetry. Actuation of an actuator may also include rotating an actuator or a portion thereof, e.g., a staging reservoir, in an arc defining a plane, such as a vertical or horizontal plane. Elements which are illustrated in FIGS. 9A and 9B and their corresponding numerical designations are summarized as follows:

TABLE 3

| Element | Numerical Designation |
|---|---|
| Sample reservoir | 901 |
| Housing | 902 |
| Proximate position of filtering element | 903 |
| Actuator | 905 |
| Staging reservoir | 906 |
| Handle | 907 |

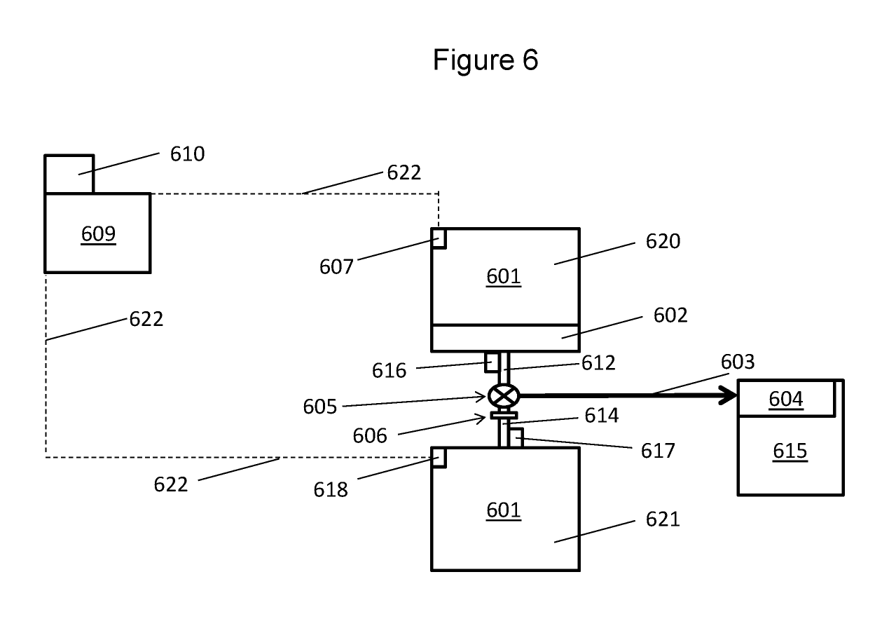

In some embodiments, the subject devices include an electrophoresis assembly and do not include an analysis assembly, such as a microfluidic assembly. A schematic of such an embodiment is shown in FIG. 6. FIG. 6 specifically illustrates an electrophoresis assembly 601 having a sample reservoir 602, and an analysis element 604. Electrophoresis assembly may also include a first buffer reservoir 620, such as a low salt buffer reservoir, and a second buffer reservoir 621, such as a high salt buffer reservoir. Sample reservoir 602 and first buffer reservoir 621 may be one and the same. Electrophoresis assembly 601 may also include connectors 612, 614, for operably, e.g., fluidically, connecting portions of the device, such as the sample reservoir 602 and the actuator 605. The device shown in FIG. 6 also includes an actuator 605 having a staging reservoir and which is actuable between a first configuration and a second configuration, wherein the staging reservoir is operably, e.g., fluidically, connected with the sample reservoir 602 when the actuator is in the first configuration. The staging reservoir may also be operably connected with the analysis element 604 when the actuator 605 is in the second configuration or operably connected, e.g., at a suitable time, with a transfer element, e.g., a container, such as a tube or pipette, for transferring, such as transferring automatically or manually, to the analysis element 604. It should be noted that no direct connection between the staging reservoir of actuator 605 and analysis element 604 is required. Instead, in some embodiments, one or more suitable intermediate devices and/or transfer elements may be utilized to transfer the analyte from the staging reservoir to analysis element 604. The transfer process is shown schematically in FIG. 6 as element 603. In some embodiments, the transfer process may occur via one or more exchangeable connectors, e.g., tubes, configured to connect the staging area 605 to, for example, an input port of one or more other, e.g., separate and independent, device configured for sample analysis, such as by analyte detection, such as a device including an analysis element 604. A detection device 615 including the analysis element 604 is shown in FIG. 6. As such, in some embodiments, the subject devices include an electrophoresis assembly operably connected, such as reversibly and/or removably coupled, to one or more detection device, such as a detection device 615 including one or more analysis element 604. In some embodiments of the subject devices, such as the device shown in FIG. 6, a detection device 615 is a separate device, e.g., a device having its components substantially contained within a different housing, than the device including the electrophoresis assembly 601.

Furthermore, in some embodiments, the subject devices may include one or more electrodes in addition to electrodes 607 and 618 within an electrophoresis assembly, such as a third electrode. An optional third electrode 616 and an additionally optional fourth electrode 617 are shown in FIG. 6. Such one or more additional electrodes may be included in, such as integral with, and/or operably attached to connectors 612 and 614, for operably, e.g., electrically, connecting portions of the device. Such one or more additional electrodes may also optionally be operably connected, such as electrically connected, to a power supply, e.g., 609, via, for example, one or more electrical connections (not shown in FIG. 6).

As is also shown in FIG. 6, the disclosed devices may include an electrophoresis assembly 601 including one or more filtering elements 606, such as a membrane, positioned downstream of an actuator 605. In some embodiments, one or more filters, such as a membrane, are removably attached to and/or replaceable within an electrophoresis assembly. The electrophoresis assembly 601 of the device shown in FIG. 6 also includes electrodes, e.g., microelectrodes, such as a first electrode 607 and second electrode 618, each operably coupled, such as electrically coupled, e.g., via electrical connections 622, to one or more power supply 609, such as a power supply 609 having a power supply controller 610, such as an electronic controller including, for example, a computer, for turning the power supply 609 on and off and controlling the voltage and/or current provided by the power supply 609. By controlling the polarity any suitable two or more of electrodes 607, 616, 617 and 618, a charged analyte can be electrophoresed in one or more steps to the staging reservoir of actuator 605. Other operable connections which are not shown are possible as well.

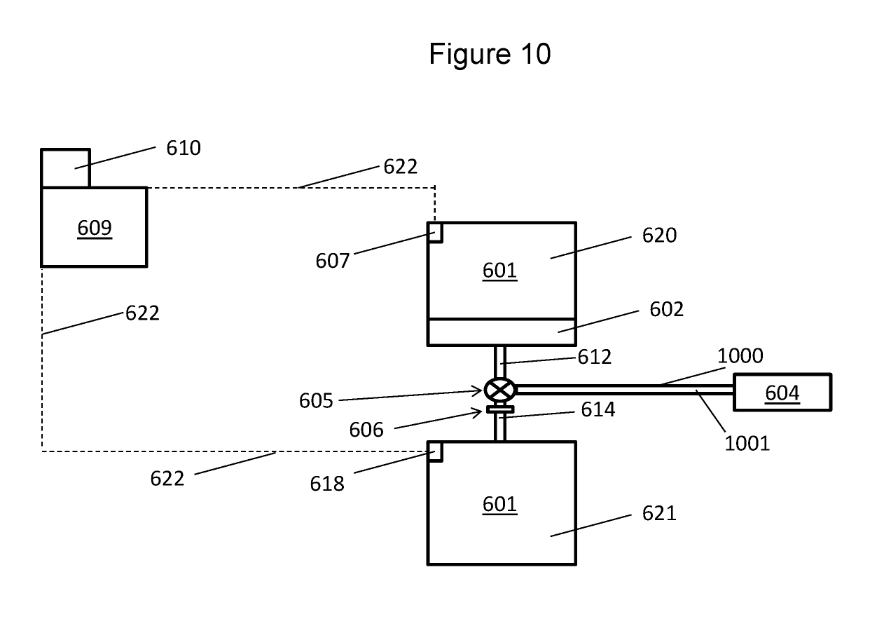

In some embodiments of the subject devices, such as the device shown in FIG. 10, an analysis element 604 is a portion of the same device as the electrophoresis assembly 601. The embodiment of the device shown in FIG. 10 includes many of the same components as shown and labelled in FIG. 6. However, the analysis area 604 of FIG. 10 is not a separate device than a device including the electrophoresis assembly 601. Instead, analysis area 604 is included in the analysis assembly 1000 of a single device and is operably connected to a staging reservoir of actuator 605 via an analysis assembly sample transfer component 1001, such as a conduit, e.g., capillary tubing. Elements which are illustrated in FIGS. 6, 10 and 11 and their corresponding numerical designations are summarized as follows:

TABLE 4

| Element | Numerical Designation |
|---|---|
| Electrophoresis assembly | 601 |
| Sample reservoir | 602 |
| Transfer process | 603 |
| Analysis element | 604 |
| Actuator | 605 |
| Filtering element(s) | 606 |
| First electrode | 607 |
| Power supply | 609 |
| Power supply controller | 610 |
| Connector | 612 |
| Connector | 614 |
| Detection device | 615 |
| Electrode(s) | 616-618 |
| First buffer reservoir | 620 |
| Second buffer reservoir | 621 |
| Electrical connections | 622 |
| Analysis assembly | 1000 |
| Sample transfer component | 1001 |
| Electrode | 1101 |

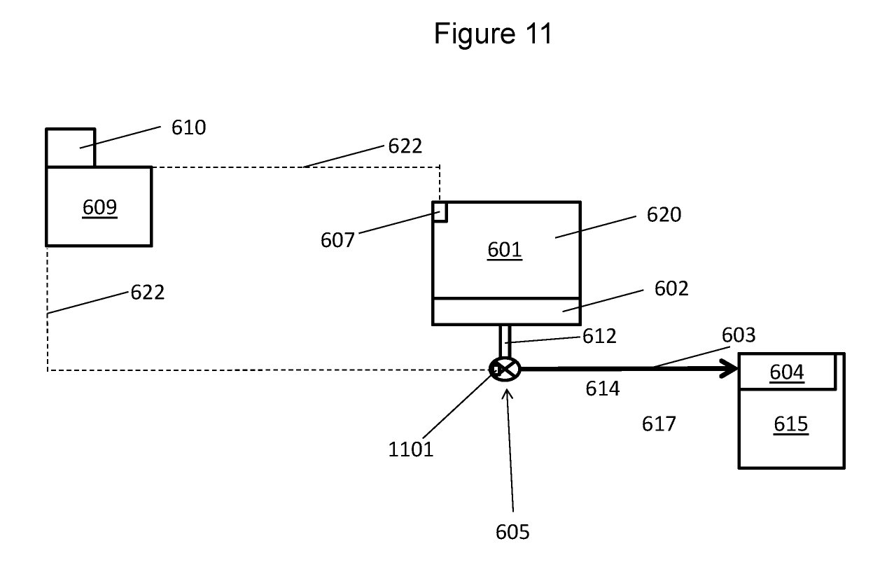

In some embodiments of the subject devices, such as the device depicted schematically in FIG. 11, the device includes an electrode 1101 within the staging reservoir of actuator 605. Electrode 1101 can be operably connected to power supply 609. As such, by controlling the polarity of electrodes 607 and 1101, a charged analyte can be electrophoresed to the staging reservoir of actuator 605. The embodiment of the device depicted schematically in FIG. 11 also includes many of the same components as shown and labelled in FIG. 6. Also, in some versions of the subject embodiments, there is no flow of fluid, such as buffer fluid. However, movement of analyte, such as a charged analyte, can occur in response to an electric field in the device. As such, in some embodiments, fluid, such as buffer fluid, remains in a first buffer reservoir 620, such as a low salt buffer reservoir, or a high salt buffer reservoir and/or a sample reservoir 602.

In some embodiments, electrophoresis assemblies of the disclosed devices do not include one or more filters, such as a membrane. In various aspects, one or more filters, such as a membrane, may be included in the actuator. As such, in some embodiments, one or more filters are removably attached to and/or replaceable within an actuator. In such embodiments, a filter is configured to actuate, e.g., rotate, with the actuator. In other words, in some embodiments, actuating the actuator, moves, such as rotates, a filter with respect to other portions of a device, such as an electrophoresis assembly.

Embodiments of the subject devices include one or more housings having an opening configured to allow passage of liquid therethrough from a first end to a second end of the housing and/or from a first end to a third end of the housing. The housing or a portion thereof, e.g., the opening, can define a sample reservoir of an electrophoresis assembly. Such a sample reservoir can be a large volume reservoir and can be shaped, for example, as a cone and/or cylinder with an opening at a first end to allow passage of liquid, such as a sample, into the housing. A sample reservoir, such as a large volume reservoir, can have any of the volumes and/or other dimensions as described herein. A sample reservoir can also include an opening at a second end, such as an opening which is smaller than the opening at the first end, for allowing passage of liquid, such as a sample, out of the sample reservoir and/or into a staging reservoir of a device. Furthermore, housings of the subject devices may include or define one or more portions of the electrophoresis assemblies and/or analysis assemblies, such as microfluidic assemblies described herein.

In some embodiments of the subject devices, a staging reservoir is operably connected to a sample reservoir, such as a large volume reservoir by a connector, such as tube configured to allow fluid flow therethrough. In some embodiments, housings of the subject devices define, and/or are operably connected to, a connector, such as a first sample conveying region, such as a conduit, between a sample reservoir and a staging reservoir. A first sample conveying region may be configured to allow passage of liquid, such as a liquid containing a sample therethrough from a sample reservoir to a staging reservoir. A first sample conveying region may have a length of, for example, 10 cm or less, such as 5 cm or less, such as 1 cm or less, such as 5 mm or less, such as 1 mm or less, such as 0.1 mm or less, 500 μm or less, 100 μm or less, 50 μm or less, 20 μm or less, 10 μm or less, 5 μm or less, or 1 μm or less, and/or inclusively ranging from 0 mm to any of these listed lengths. A first sample conveying region may have a length ranging from, for example, 1 μm to 50 cm, such as 1 μm to 20 cm, such as 1 μm to 10 cm, such as from 0.1 mm to 5 cm, such as from 0.5 mm to 3 cm, such as from 1 mm to 1 cm, each range inclusive. A first sample conveying region may have a length of, for example, 50 cm or greater, 20 cm or greater, 10 cm or greater, 5 cm or greater, 3 cm or greater, 1 cm or greater, 5 mm or greater, 3 mm or greater, or 1 mm or greater. A first sample conveying region may have a length of, for example, 50 cm or less, 20 cm or less, or 10 cm or less, or any other length or other dimensions that are compatible with the functionality of the device. In some embodiments, a first sample conveying region may have a cross-sectional diameter or radius of, for example, 5 cm or less, such as 1 cm or less, such as 0.1 cm or less, such as 0.01 cm or less, such as 0.001 cm or less. A first sample conveying region may also be part of an electrophoresis assembly. Also, in some embodiments, the devices do not include a first sample conveying region and the sample reservoir is immediately adjacent to the staging reservoir.

In various embodiments, devices according to the subject disclosure include a plurality, e.g., 2, 3, 4, 5, 10, 50, 100, 1000, or more, sample reservoirs, such as large volume reservoirs, which are each removably exchangeable and/or operably connectable to other portions of the devices, such as one or more staging reservoir. In some embodiments, at least two of the plurality of sample reservoirs have different volume capacities.

In some embodiments, housings of the disclosed devices define, and/or are operably connected to, a second sample conveying region, such as a conduit, between a staging reservoir and a filtering element. A second sample conveying region may be part of an electrophoresis assembly. Such second sample conveying region may be configured to allow passage of liquid therethrough from a staging reservoir to a filtering element. A second sample conveying region may have a length of, for example, 10 cm or less, such as 5 cm or less, such as 1 cm or less, such as 5 mm or less, such as 1 mm or less, such as 0.1 mm or less, 500 μm or less, 100 μm or less, 50 μm or less, 20 μm or less, 10 μm or less, 5 μm or less, or 1 μm or less, and/or inclusively ranging from 0 mm to any of these listed lengths. A second sample conveying region may have a length ranging from, for example, 1 μm to 50 cm, such as 1 μm to 20 cm, such as 1 μm to 10 cm, such as from 0.1 mm to 5 cm, such as from 0.5 mm to 3 cm, such as from 1 mm to 1 cm, each range inclusive. A second sample conveying region may have a length of, for example, 20 cm or greater, 10 cm or greater, 5 cm or greater, 3 cm or greater, 1 cm or greater, 5 mm or greater, 3 mm or greater, or 1 mm or greater. A second sample conveying region may have a length of, for example, 50 cm or less, 20 cm or less, or 10 cm or less, or any other length or other dimensions that are compatible with the functionality of the device. In some embodiments, a second sample conveying region may have a cross-sectional diameter or radius of, for example, 5 cm or less, such as 1 cm or less, such as 0.1 cm or less, such as 0.01 cm or less, such as 0.001 cm or less, or any other cross-sectional diameter or radius that is compatible with the functionality of the device. In some embodiments, the devices do not include a second sample conveying region and the staging reservoir and/or actuator is immediately adjacent to the filter.

In some embodiments, housings of the subject devices define, and/or are operably connected to, a third sample conveying region, such as a conduit, between a staging reservoir and a sample outlet. In some embodiments, a third sample conveying region may be oriented perpendicularly to a first sample conveying region. A third sample conveying region may also be configured to allow passage of liquid, such as a sample, therethrough from a staging reservoir to a sample outlet. A third sample conveying region may have any of the same dimensions as the first or second sample conveying regions. In some embodiments, a third sample conveying region has a length which is longer than the lengths of the first and/or second sample conveying regions. A third sample conveying region may have a length of, for example, 500 cm or less, 200 cm or less, 100 cm or less, 50 cm or less, 10 cm or less, such as 5 cm or less, such as 1 cm or less, such as 5 mm or less, such as 1 mm or less, such as 0.1 mm or less, 500 μm or less, 100 μm or less, 50 μm or less, 20 μm or less, 10 μm or less, 5 μm or less, or 1 μm or less, and/or inclusively ranging from 0 mm to any of these listed lengths. A third sample conveying region may have a length ranging from, for example, 1 μm to 500 cm, 1 μm to 200 cm, such as 1 μm to 50 cm, such as 1 μm to 20 cm, such as 1 μm to 10 cm, such as from 0.1 mm to 5 cm, such as from 0.5 mm to 3 cm, such as from 1 mm to 1 cm. A third sample conveying region may have a length of, for example, 500 cm or greater, 200 cm or greater, 100 cm or greater, 50 cm or greater, 20 cm or greater, 10 cm or greater, 5 cm or greater, 3 cm or greater, 1 cm or greater, 5 mm or greater, 3 mm or greater, or 1 mm or greater, or any other length or other dimensions that are compatible with the functionality of the device. In some embodiments, a third sample conveying region may have a cross-sectional diameter or radius of, for example, 5 cm or less, such as 1 cm or less, such as 0.1 cm or less, such as 0.01 cm or less, such as 0.001 cm or less, or any other cross-sectional diameter or radius that is compatible with the functionality of the device. In some embodiments, the third sample conveying region is configured to transfer a sample therethrough to a detection region and/or another separate device for analysis. A sample outlet may be part of and/or operably connected to an analysis assembly, such as a microfluidic assembly, and/or an analysis element. Also, in some embodiments, the devices do not include a third sample conveying region and the staging reservoir and/or actuator is immediately adjacent to a portion of the analysis assembly, such as a microfluidic assembly.

In some aspects, housings of the subject devices define a fluid conveying region, such as a conduit, between a staging reservoir and portion of an analysis assembly, such as a microfluidic assembly, such as a fluid supply for supplying non-sample containing fluid to the staging reservoir. In some embodiments, a fluid conveying region may be oriented perpendicularly to a first sample conveying region and may have any of the dimensions of the first, second, and/or third sample conveying regions described herein. A fluid conveying region may have a length of, for example, 500 cm or less, 200, cm or less, 100 cm or less, 10 cm or less, such as 5 cm or less, such as 1 cm or less, such as 5 mm or less, such as 1 mm or less, such as 0.1 mm or less, 500 μm or less, 100 μm or less, 50 μm or less, 20 μm or less, 10 μm or less, 5 μm or less, or 1 μm or less, and/or inclusively ranging from 0 mm to any of these listed lengths. A fluid conveying region may have a length ranging from, for example, 1 μm to 500 cm, such as 1 μm to 200 cm, such as 1 μm to 100 cm, such as 1 μm to 10 cm, such as from 0.1 mm to 5 cm, such as from 0.5 mm to 3 cm, such as from 1 mm to 1 cm. A fluid conveying region may have a length of, for example, 500 cm or greater, 200 cm or greater, 100 cm or greater, 20 cm or greater, 10 cm or greater, 5 cm or greater, 3 cm or greater, 1 cm or greater, 5 mm or greater, 3 mm or greater, or 1 mm or greater, or any other length or other dimensions that are compatible with the functionality of the device. A fluid conveying region may be part of and/or operably connected to an analysis assembly, such as a microfluidic assembly and/or an analysis element when, for example, an actuator is in a specific rotational configuration. In some embodiments, the devices do not include one or more of the sample and/or fluid conveying regions as described above.

In various embodiments, housings of the subject devices may define a filtering conduit, which may be part of an electrophoresis assembly, and which may be configured for operably connecting a staging reservoir and an electrophoresis assembly container configured for receiving liquid, e.g., waste liquid, therein. A filtering conduit may include a filter, such as any of the filters described herein, at a first end. Such a filter may be attached to and/or mounted in the filtering conduit by one or more retaining elements, e.g., an o-ring, such as a plastic and/or rubber o-ring. Such a filter may also be entirely contained within the housing or a portion thereof, e.g., a filtering conduit.

In some embodiments, the devices or portions thereof, e.g., an actuator, do not rotate in actuation. For example, in some embodiments, actuators of the devices are configured to slide, for example within or along a housing, between two or more configurations. In some embodiments, actuators are moved in a first direction, e.g., pushed in, and/or moved in a second direction opposite the first direction, e.g., pulled out to actuate the actuator. In such embodiments, the actuator and/or staging reservoir may slide within the housing between a first configuration, in which the staging reservoir is operably connected and/or aligned with one or more aspects of an electrophoresis assembly, such as a sample reservoir, such as a large volume reservoir, and a second configuration, in which the staging reservoir is operably connected and/or aligned with one or more aspects of an analysis assembly, such as a microfluidic assembly, such as a connector or channel thereof. In some embodiments, actuating a sliding actuator may fluidically disconnect a sample reservoir from a channel or conduit of an electrophoresis assembly and/or fluidically connect the sample reservoir with a channel or conduit of an analysis assembly, such as a microfluidic assembly. Such channels or conduits can be vertically oriented and/or horizontally oriented. Actuating the actuator can in some embodiments, disconnect and/or remove an operable connection between a sample reservoir and an electrophoresis assembly or portions thereof and/or a sample reservoir and an analysis assembly, such as a microfluidic assembly or portions thereof. Additionally, the described methods include actuating a sliding actuator by sliding the actuator from a first configuration to a second configuration and from the second configuration to the first configuration, wherein in the first configuration, a staging reservoir of the actuator is operably connected with an electrophoresis assembly and in the second configuration, the staging reservoir is operably connected with an analysis assembly, such as a microfluidic assembly. Other embodiments may also include one, two, three, five, or ten or more additional configurations, such as an, additional, e.g., third, configuration in which such a staging reservoir is operably connected with a second analysis assembly, such as a second microfluidic assembly and/or an additional configuration in which a staging reservoir is operably connected with a second electrophoresis assembly.

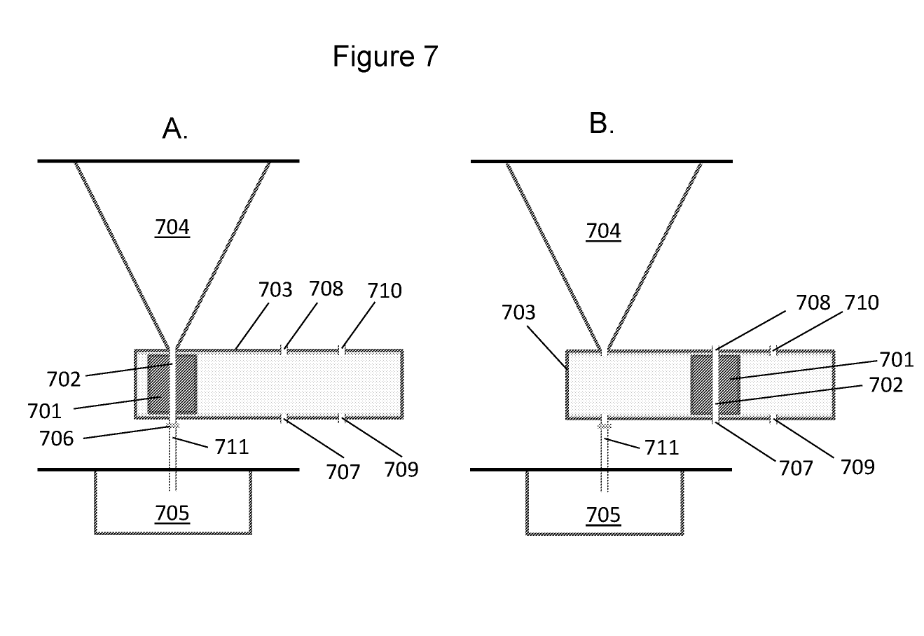

A cross-sectional view of an embodiment of a device including an actuator configured to slide, for example within or along a housing, between two or more configurations is shown in FIGS. 7A and 7B. FIGS. 7A and 7B show, at least in part, an electrophoresis assembly which may have any of the characteristics of the electrophoresis assemblies described herein. FIGS. 7A and 7B also depict an actuator 701 including a staging reservoir 702 and which is slidable in a housing 703. Housing 703 may have any of the characteristics of the housings described herein. The device shown in FIGS. 7A and 7B also includes a first reservoir 704 and a second reservoir 705 of an electrophoresis assembly. First reservoir 704 and a second reservoir 705 may have any of the same characteristics as any of the reservoirs of electrophoresis assemblies described herein. Also show in FIG. 7 is a filtering element 706, which may have any of the characteristics of the filtering elements described herein. The actuator 701 of the device shown in FIGS. 7A and 7B is movable, e.g., slidable, between a first configuration, as shown in FIG. 7A, a second configuration, shown in FIG. 7B, and a third configuration (not shown). An actuator which is configured to slide, such as slide along a housing, at least between a first configuration and a second configuration is referred to herein as a “sliding actuator”. One embodiment of a sliding actuator is provided in FIG. 7. In the first configuration, the staging reservoir 702 is operably connected to the first reservoir 704 and second reservoir 705 of the electrophoresis assembly. In the first configuration, the sample may be concentrated in the staging reservoir 702, as described throughout this disclosure. In the second configuration, the staging reservoir 702 is operably connected to a first fluidic channel 707 and/or a second fluidic channel 708. The first fluidic channel 707 and/or second fluidic channel 708 may in turn be configured to allow access to the sample in the staging reservoir 702 and for the sample to be removed from the staging reservoir 702, e.g., removed by automatically and/or manually transferring out of the staging reservoir 702 into another element, such as a tube or well, or a separate detection device. In some embodiments, the first fluidic channel 707 and/or second fluidic channel 708 may be operably connected, for example, via a connector, to a detection device and/or analysis element as described herein. In the third configuration, the staging reservoir 702 is operably connected to a third fluidic channel 709 and/or a fourth fluidic channel 710. The third fluidic channel 709 and/or fourth fluidic channel 710 may in turn be configured to allow access to the sample in the staging reservoir 702 and for the sample to be removed from the staging reservoir 702, e.g., removed by automatically and/or manually transferring out of the staging reservoir 702 into another element, such as a tube or well. In some embodiments, the third fluidic channel 709 and/or fourth fluidic channel 710 is operably connected, for example, via a connector, to a detection device and/or analysis element as described herein. In further configurations, which are not shown, the actuator 701 of the device is movable, e.g., slidable, between a first configuration, as shown in FIG. 7A, and configurations additional to the second and third configurations described above. In addition, in some embodiments, the staging reservoir 702 may be operably, e.g., fluidically, connected via a connector 711, e.g., a lower connector, to a second reservoir 705 when, for example, the actuator 701 is in a particular configuration, e.g., a configuration as shown in FIG. 7A, but not another configuration, e.g., a configuration as shown in FIG. 7B. Elements which are illustrated in FIGS. 7A and 7B and their corresponding numerical designations are summarized as follows:

TABLE 5

| Element | Numerical Designation |
|---|---|
| Actuator | 701 |
| Staging reservoir | 702 |
| Housing | 703 |
| First reservoir | 704 |
| Second reservoir | 705 |
| Filtering element | 706 |
| First fluidic channel | 707 |
| Second fluidic channel | 708 |
| Third fluidic channel | 709 |
| Fourth fluidic channel | 710 |
| Connector | 711 |

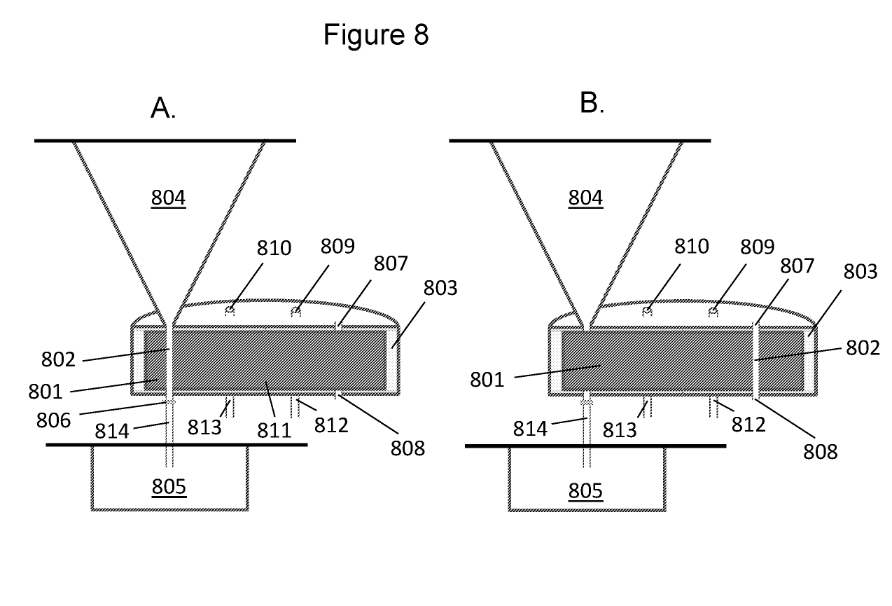

A cross-sectional view of an embodiment of a device including an actuator configured to rotate, for example within a housing, between two or more configurations is shown in FIGS. 8A and 8B. FIGS. 8A and 8B show, at least in part, an electrophoresis assembly which may have any of the characteristics of electrophoresis assemblies described herein. FIGS. 8A and 8B also depict an actuator 801 including a staging reservoir 802 and which is rotatable, e.g., rotatable in a horizontal plane e.g., a plane parallel to a ground surface, in a housing 803, such as a cylindrically shaped housing. As noted above, in some embodiments, actuation includes rotating an actuator or a portion thereof along an arc length which defines a vertical or horizontal plane. In some embodiments, actuators, such as the actuator 801 shown in FIG. 8, and/or housings, such as housing 803, may have a round, and/or cylindrical shape and/or may be shaped as a disk and/or drum. An actuator having a disk shape and configured to rotate about a vertical axis is referred to herein as a “disk actuator”. One embodiment of a disk actuator is provided in FIG. 8. Various embodiments of actuators may have a plurality, such as two, three, four, five, or more staging reservoirs therein, each of which is defined, at least in part, by one or two openings in the actuator. In some embodiments, actuators may be configured to rotate about an axis, e.g., an axis of symmetry, such as a vertical axis 811 or a horizontal axis. Housing 803 may have any of the characteristics of the housings described herein. The device shown in FIGS. 8A and 8B also includes a first reservoir 804 and a second reservoir 805 of an electrophoresis assembly. First reservoir 804 and a second reservoir 805 may have any of the same characteristics as any of the reservoirs of the electrophoresis assemblies described herein. Also show in FIG. 8 is a filtering element 806, which may have any of the characteristics of the filtering elements described herein. The actuator 801 of the device shown in FIGS. 8A and 8B is movable, e.g., rotatable, between a first configuration, as shown in FIG. 8A, a second configuration, shown in FIG. 8B, a third configuration (not shown), and a fourth configuration. In the first configuration, the staging reservoir 802 is operably connected to the first reservoir 804 and second reservoir 805 of the electrophoresis assembly. In the first configuration, the sample may be concentrated in the staging reservoir 802, as described throughout this disclosure. In the second configuration, the staging reservoir 802 is operably connected to a first fluidic channel 807 and/or a second fluidic channel 808. The first fluidic channel 807 and/or second fluidic channel 808 may in turn be configured to allow access to the sample in the staging reservoir 802 and for the sample to be removed from the staging reservoir 802, e.g., removed by automatically and/or manually transferring out of the staging reservoir 802 into another element, such as a tube or well, or a separate detection device. In some embodiments, the first fluidic channel 807 and/or second fluidic channel 808 may be operably connected, for example, via a connector, to a detection device and/or analysis element as described herein. In the third configuration, the staging reservoir 802 is operably connected to a third fluidic channel 809 or a fourth fluidic channel 810. In the third configuration, the staging reservoir 802 may also be operably connected to a fifth fluidic channel 812 or a sixth fluidic channel 813 each of which may optionally be included in the device. The third fluidic channel 809, or fourth fluidic channel 810, and optionally, fifth fluidic channel 812 and/or sixth fluidic channel 813, may in turn be configured to allow access to the sample in the staging reservoir 802 and for the sample to be removed from the staging reservoir 802, e.g., removed by automatically and/or manually transferring out of the a staging reservoir 802 into another element, such as a tube or well. In some embodiments, the third fluidic channel 809 and/or fourth fluidic channel 810, and optionally, the fifth fluidic channel 812 and/or sixth fluidic channel 813, is operably connected, for example, via a connector, to a detection device and/or analysis element as described herein. In further configurations, which are not shown, the staging reservoir 802 of the actuator 801 of the device is movable, e.g., rotatable, between a first configuration, as shown in FIG. 8A, and configurations additional to the second and third configurations described above. Furthermore, in some embodiments, the staging reservoir 802 may be operably, e.g., fluidically, connected via a connector 814, e.g., a lower connector, to a second reservoir 805 when, for example, the actuator 801 is in a first configuration, e.g., a configuration as shown in FIG. 8A, but not a second configuration, e.g., a configuration as shown in FIG. 8B. Elements which are illustrated in FIGS. 8A and 8B and their corresponding numerical designations are summarized as follows:

TABLE 6

| Element | Numerical Designation |
|---|---|
| Actuator | 801 |
| Staging reservoir | 802 |
| Housing | 803 |
| First reservoir | 804 |
| Second reservoir | 805 |
| Filtering element | 806 |
| First fluidic channel | 807 |
| Second fluidic channel | 808 |
| Third fluidic channel | 809 |
| Fourth fluidic channel | 810 |
| Vertical axis | 811 |
| Fifth fluidic channel | 812 |
| Sixth fluidic channel | 813 |
| Connector | 814 |

Housings and/or electrophoretic assemblies or portions thereof, and/or analysis assemblies, such as a microfluidic assemblies, or portions thereof, and/or actuators and/or portions thereof, of the subject devices may be made of any material with properties, e.g., resistance to corrosion, and/or durability, which allow the housing to carry out its function within the devices as described herein. For example, housings may be composed of one or more metallic materials, such as stainless steel, and/or polymeric materials or combinations thereof, such as rubber and/or plastic. Further, components described herein, such as housings and/or electrophoretic assemblies or portions thereof, and/or analysis assemblies, such as microfluidic assemblies or portions thereof, and/or actuators and/or portions thereof, of the subject devices may be composed fully or partially of one or more of polystyrene, polycarbonate, polypropylene, parylene-C, Polyether ether ketone (PEEK), Polytetrafluoroethylene (PTFE), Ethylene ChloroTriFluoroEthylene (ECTFE), Poly(methyl methacrylate) (PMMA), Polydimethylsiloxane (PDMS), Polyphenylsulfone (PPSF), or any combinations thereof.

In various embodiments, the subject devices include a housing having an opening therein configured for receiving at least a portion of an actuator and/or for allowing the received portion of the actuator to rotate therein. Such an opening may be shaped, for example, as a cylinder and may be configured for receiving, e.g., receiving therein, a reciprocating cylindrically shaped portion of the actuator, such as a portion including a staging reservoir. In various embodiments, housings of the subject devices are stationary while a portion of an actuator is moved, e.g., rotated, therein. In some aspects, housings are operably connected with an actuator or apportion thereof. For example, in some embodiments, a housing is reciprocally coupled to an actuator such that a liquid-tight seal is formed between the actuator and housing such that liquid cannot enter or exit the housing or actuator except through conduits within the housing as described herein.

In some embodiments, actuators as described herein are configured to rotate, such as rotate about an axis, such as a single axis, such as an axis of symmetry. In some embodiments, actuators are configured to rotate, e.g., rotate within a housing, 360° or more, 360° or less, such as 180° or less, such as 90° or less, such as 45° or less, such as 30° or less, such as 15° or less, or in accordance with any of the actuations described below. Embodiments of actuators can be configured to actuate in an inclusive range from 0°-15°, 15°-20°, 20°-25°, 25°-30°, 30°-35°, 35°-40°, 40°-45°, 45°-50°, 50°-55°, 55-60°, 65-70°, 70°-75°, 75-80°, 80°-85°, 85°-90°, 90°-95°, 95°-100°, 100°-120°, 120°-170°, 175°-180°, 180°-185°, 185°-190°, 190°-360°, or from 0° up to the upper or lower limit of any of these provided ranges, and an actuation of the device can be in any of these rotational amounts. Such an actuation can be in a single direction, e.g., a single rotational direction, and a device may be configured to also actuate, such as by rotating, an amount in any of the listed ranges in the opposite direction. In some embodiments, actuators are configured to rotate bidirectionally and/or unidirectionally, e.g., only in a single direction. In various aspects, actuators are symmetrical with respect to one or two planes, such as planes of symmetry. Furthermore, in some embodiments, actuating the actuator as described herein may include rotating the actuator or a portion thereof, e.g., an end portion, 360° or less, such as 180° or less, such as 90° or less, such as 45° or less, such as 30° or less, such as 15° or less in a single rotational direction and/or in two opposite rotational directions, e.g., rotating an amount in a first direction and then an equal amount in a second direction opposite the first. In some embodiments, actuating the actuator as described herein may include rotating the actuator or a portion thereof, e.g., an end portion, in a radius extending from 0° to 360°, inclusive, such as from 0° to 180°, such as from 0° to 90°, such as from 0° to 45°, such as from 0° to 30°, such as from 0° to 15°, each inclusive, in a first rotational direction and/or in a second rotational direction opposite the first. In some embodiments, actuating the actuator includes rotating the actuator between the first and second configurations.

As noted above, in some embodiments, actuators of the subject devices have a one or more portions, such as one or more handle, such as a valve handle, such as a handle shaped as a rectangular plate, configured for manual actuation of the actuator. In some embodiments, manual actuation includes an operator physically touching an actuator and exerting force on the actuator to actuate, e.g., move, the actuator or a portion thereof. In some embodiments, actuators have an end portion, such as a portion at an end of the actuator which is opposite from an end of the actuator having the handle. In some embodiments, actuators have a connecting portion connecting an end portion to a handle portion of an actuator. Such a connecting portion may be shaped as a cylinder and/or may be fixedly connected and/or integral with an end portion and/or handle of an actuator.

Embodiments of the disclosed devices also include actuators which are automatic actuators. Automatic actuators can include an automatic controller, such as an electronic controller, such as a computer controller, for controlling actuation of the actuator. Automatic actuators, in some embodiments, are not manually operated actuators and are configured to operate without an operator performing a manual actuation of the device, such as by rotation. In various embodiments, automatic actuators may include an interface, such as an input, e.g., buttons and/or switches, for receiving instructions, such as instructions designating when and/or how an actuator will actuate, from an operator. Automatic actuators may provide automatic actuation between, for example, first and second configurations as described herein, one or a plurality of times. Such automatic actuation can be actuation in a first direction, such as a first rotational direction, and/or a second direction multiple times and/or in varying degrees. Embodiments of automatic actuators can also provide actuation on a schedule set by a controller, such as a schedule that designates actuation or patterns of actuation that occur on regular or irregular intervals. Automatic actuators may also be configured to actuate based on an input from a separate source, such as one or more sensors, e.g., analyte and/or temperature sensors, or a timer of a device. Embodiments of automatic actuators can also include a display for producing an output providing information relating to actuation for an operator. Automatic actuators can include wireless capabilities, for example, for wireless activation and/or deactivation. Embodiments of automatic actuators can also be operated via a mobile device, such as a mobile telephone. Automatic actuators may also include one or more computer and/or power source, such as a battery, and/or can be operatively coupled to a power source. Furthermore, automatic actuators may include one or more propelling element, such as a motor, such as an electric motor, to move the actuator in actuation and/or to drive actuation of the actuator in one or more directions, e.g., a first direction, such as a rotational or linear direction, and/or a second direction opposite the first direction.

An actuator or a portion thereof, e.g., an end portion of an actuator, may be configured to be received into and/or fully retained within and/or rotated within a housing of a device. An end portion of an actuator may include the staging reservoir therein and/or define the edges of the staging reservoir. An end portion of an actuator may also be shaped substantially as a cylinder.

Actuators or portions thereof, e.g., an end portion of an actuator, may include and/or define a staging reservoir therein. Such a staging reservoir may be a passage, e.g., a cylindrical container or conduit, such as a tube, extending from a first opening at a first side of an end portion of an actuator, through the end portion, to a second opening at a second side of an end portion of an actuator opposite the first side. The end portion of the actuator may be actuable such that in a first configuration the first opening and second openings are operably connected, e.g., fluidically connected, to the electrophoresis assembly and in a second configuration, after the end portion is rotated, the first opening, or the second opening is operably connected, e.g., fluidically connected, to an analysis assembly, such as a microfluidic assembly and the opening which is not operably connected to the assembly is operably connected to a fluid conveying region including, for example, a non-sample containing fluid source and/or is sealed, for example, by a surface of the housing. A staging reservoir may also be symmetrical about one or two planes and/or about an axis, such as an axis of symmetry, running down the center of the staging reservoir. In some embodiments, actuators of the subject devices are valves, e.g., fluidic valves, such as manually and/or automatically rotatable valves.

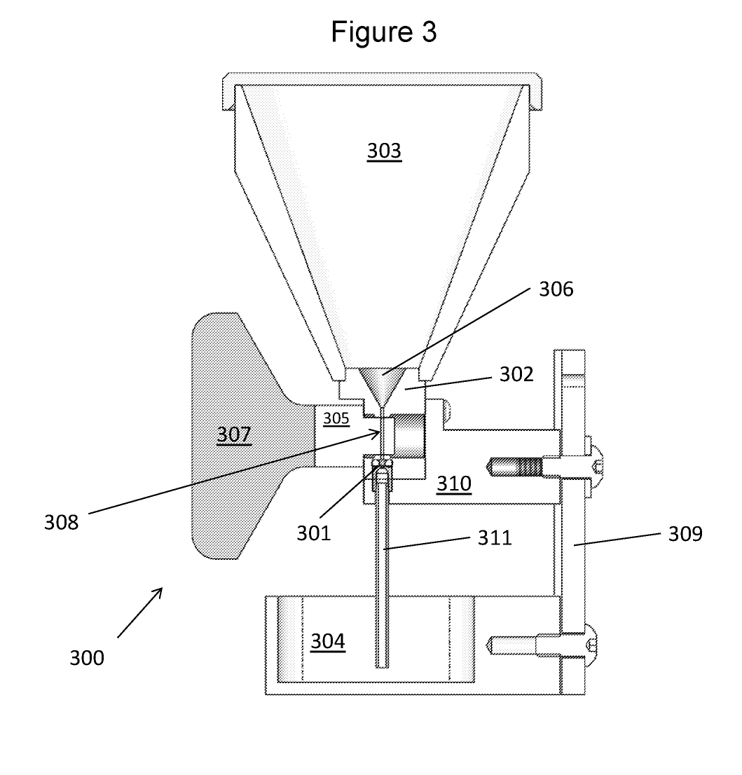

A different view of the embodiment of the portion of the device shown in FIG. 2 is shown in FIG. 3. FIG. 3 provides a portion 300 of a device having an electrophoresis assembly including a first reservoir 303, such as a low salt buffer reservoir, and a second reservoir 304, such as a high salt buffer reservoir. The first reservoir may be operably connected, e.g., fluidically connected, to a sample reservoir 306. In some embodiments, first reservoir 303 and sample reservoir 306 are one and the same. The first reservoir may also be operably connected to the second reservoir when an actuator is in a first configuration and/or not be operably connected to the second reservoir when the actuator is in the second configuration. In some embodiments, the staging reservoir 308 is operably, e.g., fluidically, connected via a connector 311 to the second reservoir when, for example, the actuator 305 is in a particular configuration, e.g., a first and/or second configuration.

FIG. 3 also depicts a housing 302 of the device defining a sample reservoir 306 and having a filtering element 301, such as a membrane, therein. Also shown is an actuator 305 including a staging reservoir 308 and a handle 307. The housing 302 is also connected to a stable support 309 by an anchor element 310. Furthermore, FIG. 3 depicts staging reservoir 308 in a first configuration wherein the staging reservoir 308 is operably, e.g., fluidically, connected to sample reservoir 306. In other embodiments, devices of the subject embodiments do not include a second reservoir, filtering element or connector connecting a staging reservoir to a second reservoir. In such embodiments, for example, the device is configured such that fluid, such as buffer fluid does not flow through the staging reservoir. Rather, analyte migrates within a fluid, such as a buffer fluid, for example, in response to an electric field, and becomes concentrated in the staging reservoir. Elements which are illustrated in FIG. 3 and their corresponding numerical designations are summarized as follows:

TABLE 7

| Element | Numerical Designation |
|---|---|
| Filtering element | 301 |
| Housing | 302 |
| First reservoir | 303 |
| Second reservoir | 304 |
| Actuator | 305 |
| Sample reservoir | 306 |
| Handle | 307 |
| Staging reservoir | 308 |
| Stable support | 309 |
| Anchor element | 310 |
| Connector | 311 |

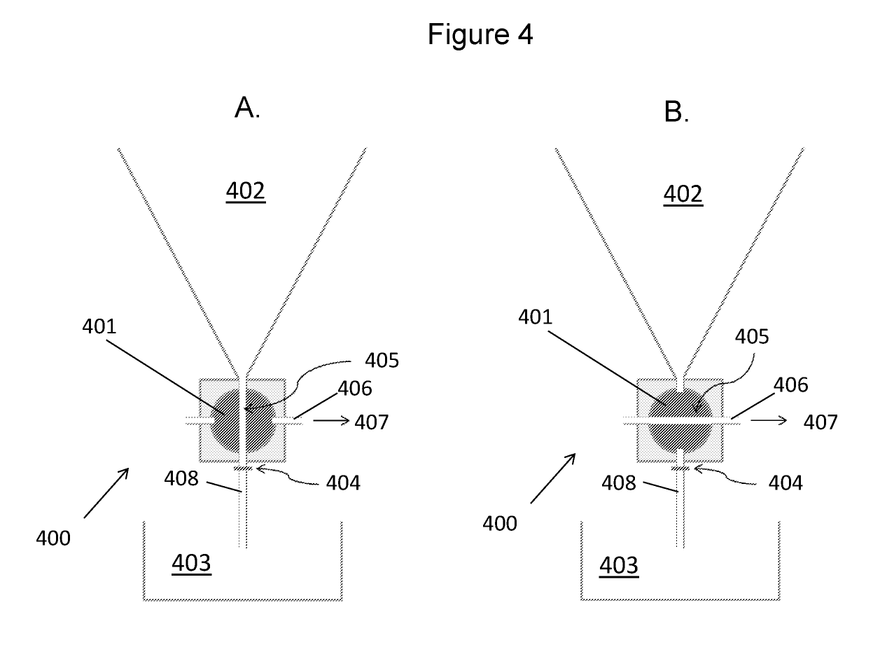

FIGS. 4A and 4B provide schematic representations of an embodiment of a device according to the present disclosure. The represented device has an electrophoresis path which is separate from an analyte detection area, such as an analysis element, and may be configured to transfer, e.g., directly transfer, a concentrated analyte to an analysis element of, for example, an analysis assembly, such as a microfluidic assembly and/or a detection device, such as a separate detection device. FIG. 4A depicts a device having an actuator 401 in a first configuration whereas FIG. 4B depicts the device with the actuator 401 in a second configuration. The actuator 401 also includes a staging reservoir 405. FIGS. 4A and 4B also show an electrophoresis assembly 400 having a first reservoir 402, such as an upper reservoir, e.g., a sample reservoir, and a second reservoir 403, such as a lower reservoir, as well as a filtering element 404, e.g., a membrane. In some embodiments, the staging reservoir 405 may be operably, e.g., fluidically, connected via a connector 408, e.g., a lower connector, to a second reservoir 403 when, for example, the actuator 401 is in a first configuration, e.g., a configuration as shown in FIG. 4A, but not a second configuration, e.g., a configuration as shown in FIG. 4B. Methods of operation of the embodiment of the device is shown in FIGS. 4A and 4B are described in further detail below. Also, in some embodiments, devices of the subject embodiments do not include a second reservoir, filtering element or connector connecting a staging reservoir to the second reservoir. In such embodiments, for example, the device is configured such that fluid, such as buffer fluid does not flow through the staging reservoir. Rather, analyte migrates within a fluid, such as a buffer fluid, for example, in response to an electric field, and becomes concentrated in the staging reservoir. Elements which are illustrated in FIG. 4 and their corresponding numerical designations are summarized as follows:

TABLE 8

| Element | Numerical Designation |
|---|---|
| Actuator | 401 |
| First reservoir | 402 |
| Second reservoir | 403 |
| Filtering element | 404 |
| Actuator | 405 |
| Fluidic channel | 406 |
| Analysis element/Detection area | 407 |
| Connector | 408 |

FIG. 1 is provided to aid in the understanding of the invention. It will be appreciated that FIG. 1 is for illustration and is not intended to limit the invention in any fashion. The system shown in FIG. 1 includes an electrophoresis assembly 101 including a sample reservoir 102, e.g., a large volume reservoir (LVR), into which a sample containing an analyte at low concentration can be introduced, and an actuator 105 including a staging reservoir integral to, for example, the electrophoresis assembly and/or an analysis assembly, such as a microfluidic assembly 103, a connector 112, such as a tube, such as a tube defined by a housing, through which an analyte in the sample reservoir 102, can be electrophoretically transported to the staging reservoir. The device of FIG. 1 also includes a microfluidic assembly 103 and an analysis element 104 in which an analyte or one or more properties thereof can be detected. The term “microfluidic chip” may be used herein to describe an analysis assembly, such as a microfluidic device, wherein the analysis element is a “chip”.

The analysis element 104 may include one or more microfluidic channels through which analyte-containing fluid can flow and/or which may contain an aqueous buffer solution through which analyte can be transported. Alternatively, the analysis element 104 can be a well or chamber including immobilized capture agents. In some embodiments, at least a portion of a housing or material surrounding the analysis element 104 is transparent, to facilitate detection of signal from the analysis element, e.g., fluorescence emissions. In some embodiments, analysis elements are integral with or part of an analysis assembly, such as a microfluidic assembly. In other embodiments, analysis elements are separate from, e.g., reversibly disconnectable and/or not integral with, an analysis assembly. The device of FIG. 1 also includes one or more filtering element 106, such as a membrane, within the electrophoresis assembly 101 configured for blocking the flow of an analyte and/or separation of an analyte from other components of a sample. In some aspects, a membrane is positioned downstream of a staging reservoir of an actuator 105 and/or is configured to concentrate an analyte in the staging reservoir by inhibiting flow of analyte through the membrane while allowing a flow of non-analyte containing fluid therethrough. In some embodiments of the electrophoresis assemblies, a filtering element includes one or more of, an exclusion gel, a viscous medium, and a sieve. The microfluidic channels within the microfluidic assembly 103 through which analyte is transported into the analysis element, in some embodiments of the subject devices, does not contain exclusion gels, a viscous medium, filters, sieves, etc. for separation of analyte.

As noted above, embodiments of the devices may include one or more filtering elements, also referred to herein as a filter. Filtering elements may include one or more membranes. A filtering element may be composed of a material selected based on one or more characteristics of the analyte to be concentrated (and/or one or more characteristics of one or more materials bound to an analyte), e.g., molecular weight, charge, etc. Thus, for example, the pore size of a membrane or the size exclusion characteristics of a gel may be selected based on the size or other characteristics of the analyte to be concentrated. Filtering elements, e.g., membranes, with a porosity that prevents the passage of molecules larger than a certain size can be selected based on the size and properties of the analyte of interest. In some embodiments, a filtering element prevents and/or substantially inhibits one or more analyte of interest from flowing therethrough but allows other substances, e.g., a solution in which an analyte is suspended such as a buffer solution, to flow therethrough. In some embodiments, the subject membranes include a plurality of pores which in turn allow the membranes to have a characteristic porosity and/or permeability. Furthermore, in some embodiments, the filtering element's, e.g., membrane's, porosity is such that it permits the electro-osmotic passage of small ions and molecules but prevents the passage of the larger analyte molecules. In other words, a filtering element, e.g., membrane, according to the subject embodiments may be configured to inhibit movement of an analyte through the filtering element.

Filtering elements, e.g., membranes, according to the subject embodiments may have a molecular weight cut off (MWCO) raging from, for example, 1,000 daltons to 10,000,000 daltons, such as from 1,000 daltons to 5,000,000 daltons, such as from 1,000 daltons to 1,000,000 daltons, 1,000 daltons to 500,000 daltons, such as from 1,000 daltons to 100,000 daltons, such as from 5,000 daltons to 100,000 daltons, 10,000 daltons to 100,000 daltons, or 50,000 daltons to 100,000 daltons. For example, a suitable filtering element may have a MWCO of about 1,000 daltons, about 2,000 daltons, about 3,000 daltons, about 4,000 daltons, about 5,000 daltons, about 6,000 daltons, about 7,000 daltons, about 8,000 daltons, about 9,000 daltons, about 10,000 daltons, about 15,000 daltons, about 20,000 daltons, about 30,000 daltons, about 40,000 daltons, about 50,000 daltons, about 60,000 daltons, about 70,000 daltons, about 80,000 daltons, about 90,000 daltons, about 100,000 daltons, about 250,000 daltons, about 500,000 daltons, about 1,000,000 daltons, about 5,000,000 daltons, or about 10,000,000 daltons. Filtering elements, e.g., membranes, according to the subject embodiments, may also have a molecular weight cut off (MWCO) which is 100,000 daltons or lager, 500,000 daltons or lager, 1,000,000 daltons or lager, 5,000,000 daltons or lager, or 10,000,000 daltons or lager. Filtering elements according to the subject embodiments may be composed, e.g., composed in full or in part, of any suitable materials including, e.g., cellulose, such as reconstituted cellulose and/or regenerated cellulose, polypropylene, such as hydrophilic polypropylene, nylon, such as modified nylon, or polyethersulfone, such as modified polyethersulfone, or any combinations thereof.

Also, as shown in FIG. 1, electrodes, such as microelectrodes 108, are positioned so that when an electric potential is applied to the electrodes, e.g., a positive charge is applied to one electrode and a negative charge is applied to another, charged analyte is transported through a solution to the appropriate region of the device, such as a staging reservoir and/or analysis element. The same is true for microelectrodes 107 shown in FIG. 1. Additional electrodes may optionally be positioned to transport analyte into and/or out of an area of the device, e.g., the analysis element 104. In some embodiments, electrodes within or proximate to the staging reservoir are not included. In addition, electrodes can be integrated into, e.g., positioned within, the LVR and/or staging reservoir or may be external electrodes placed into a portion of a device such as a sample reservoir and/or staging reservoir chamber, in contact with a sample, such as a sample including an analyte, or other solution. Power supply 109 is any power supply or source of electric current suitable for electrophoresis, e.g., one or more battery.

Analysis elements according to the subject embodiments may be included in analysis assemblies, such as microfluidic assemblies, or may be included in one or more separate, e.g., unattached and/or independent, devices, such as detection devices, which are operably and, in some embodiments reversibly, connectable to aspects of the subject devices, such as one or more staging reservoir. In other words, all the embodiments of analysis elements described or illustrated as part of analysis assemblies, such as microfluidic assemblies, may also alternatively be present in one or more other devices or device portions, such as detection devices, which are separate from the analysis or microfluidic assemblies. Analysis elements may be configured for detecting an analyte and/or analyzing properties of a sample, e.g., a processed sample, such as one or more characteristics of an analyte in a sample. Analysis elements may be one or more analysis instruments, e.g., separate and independently operating instruments, and may be configured to directly or indirectly measure or detect an analyte or a characteristic of an analyte or a component in a processed sample. Analysis elements may be configured to detect an analyte or analyze properties of a sample by performing one or more assays including, for example: well-plate based assays, enzyme-linked immunosorbent assay (ELISA), immunoassays, colorimetric assays, phosphogenic assays, luminogenic assays, fluorimetric assays, radionuclide-based assays, Nuclear magnetic resonance (NMR), spectrometry (e.g., mass spectrometry (MS)), capillary electrophoresis (CE), capillary zone electrophoresis (CZE), Polyacrylamide gel electrophoresis (PAGE), gel electrophoresis, biosensor-based device assays, fluidic device assays including lab-on-chip, lateral flow assays, passive flow assays, chromatography, quantitative polymerase chain reaction (qPCR), polynucleotide sequencing, e.g., DNA sequencing or RNA sequencing, or any combinations thereof. As such, detection devices according to the subject embodiments, may be devices configured to detect an analyte or analyze properties of a sample by using any appropriate methods or assays and may include one or more components necessary to do so, e.g., one or more biosensor. Detection devices according to the subject embodiments may also include one or more reservoir, such as a tube or well, which may be operably connected to a staging reservoir as described herein. For example, actuation of an actuator, such as actuation to a second configuration, may operably connect a staging reservoir to a reservoir of a detection device.

Analysis elements, according to various embodiments of the disclosed devices and methods, can include “capture agents” associated with a substrate in the analysis element of a device. Capture agents, which are discussed in further detail below, are agents that specifically bind to an analyte, a carrier molecule, an ionic moiety, and/or other molecule associated with the analyte. Examples of capture agents include antibodies and polynucleotides. In some instances, the capture agents are immobilized at a “capture site” or one or more capture sites in an analysis element. As used herein, the term “capture site” refers to a physical surface of an analysis element, e.g., an analysis element of an analysis assembly, such as a microfluidic assembly and/or microfluidic device, that is modified to include at least one capture agent, e.g., at least two capture agents, such as an array of capture agent molecules. Alternatively, in other embodiments, the analysis element does not include a capture agent and the sample is analyzed, e.g., detected, via other suitable means, for example, as it flows through a detector.

As is described in further detail below, in the operation of the subject devices, a sample solution, such as an aqueous liquid that contains, or is suspected of containing the analyte, is introduced into one or more sample reservoirs 102, e.g., a large volume reservoir. A sample reservoir 102, can be one or more containers, such as any of a variety of containers for holding liquids, including but not limited to: a test tube, a microfuge tube, a well of a microwell plate, a tissue culture dish, and the like. In some embodiments, a sample, e.g., an unprocessed sample, and/or a reservoir, such as a sample reservoir, such as a large volume reservoir, may have a volume of a size listed above or in a range listed above. In addition, the volume and/or liquid capacity of a sample reservoir, such as a large volume reservoir, can range from, for example, 0.1 microliter to 100 mL, such as from 0.1 microliter to 10 mL, such as from 0.1 microliter to 1 mL, such as from 0.1 microliter to 10 microliters, such as 0.1 microliter to 1 microliter. The volume and/or liquid capacity of a sample reservoir can also range from, for example, 1 microliter to 100 mL, such as from 10 microliters to 100 mL or 1 microliter to 10 mL. The volume and/or liquid capacity of a sample reservoir can also range from, for example, 100 microliters to 2 mL or 5 microliters to 1 mL. The volume and/or liquid capacity of a sample reservoir can also range from, for example, 1 nanoliter to 500 microliters, such as from 1 nanoliter to 100 microliters, such as from 1 nanoliter to 10 microliters, such as from 1 nanoliter to 5 microliters, such as from 1 microliter to 10 microliters. The volume and/or liquid capacity of a sample reservoir can also range from, for example, 0.1 microliter to 100 microliters, such as from 5 microliters to 100 microliters, such as from 10 microliters to 100 microliters, or may range from 0.1 microliter to 10 microliters. The liquid capacity and/or volume of a sample reservoir can also range from, for example, 1 picoliter to 10 microliters, such as from 1 picoliter to 5 microliters, such as from 1 picoliter to 2 microliters. All of such listed ranges are inclusive. The volume and/or liquid capacity of a sample reservoir can also be, for example, 5000 microliters or less, such as 1000 microliters or less, such as 500 microliters or less, such as 200 microliters or less, such as 100 microliters or less, such as 50 microliters or less, such as 25 microliters or less, such as 10 microliters or less, such as 1 microliter or less. The volume and/or liquid capacity of a sample reservoir can be, for example, 1 microliter or greater, such as 5 microliters or greater, such as 10 microliters or greater, such as 25 microliters or greater, such as 50 microliters or greater, such as 100 microliters or greater, such as 200 microliters or greater, such as 500 microliters or greater, such as 1000 microliters or greater, such as 5000 microliters or greater. The volume and/or liquid capacity of a sample reservoir can also be, for example, greater than 1 microliter, greater than 10 microliters, or greater than 100 microliters. The liquid capacity and/or volume of a sample reservoir can also be, for example, less than 1 microliter, less than 10 microliters, or less than 100 microliters. In addition, an unprocessed sample may have any of the volumes provided herein for a sample reservoir, such as an LVR.

In various embodiments, one or more sample reservoirs, e.g., LVRs, can be associated with, such as operably connectable to, one or more staging reservoir. In some embodiments 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 175, 200, 250 or more sample reservoirs, are included in a single device. For example, if a microtiter plate has 100 wells, the number of sample reservoirs, e.g., large volume reservoirs, can be 100.

In addition, samples, e.g., unprocessed or processed samples, of the subject disclosure may have a variety of volumes including all of the volumes listed above. In addition, the volume of a sample, such as an unprocessed sample or a processed sample, can range from, for example, 0.1 microliter to 100 mL, such as from 0.1 microliter to 10 mL, such as from 0.1 microliter to 10 microliters or from 5 microliters to 1 mL. The volume of a sample, such as an unprocessed sample or a processed sample, can also range from, for example, 1 microliter to 100 mL, such as from 10 microliters to 100 mL or 1 microliter to 10 mL, or from 1 nanoliter to 5 microliters. The volume of a sample, such as an unprocessed sample or a processed sample, can also range from, for example, 100 microliters to 2 mL. The volume of a sample, such as an unprocessed sample or a processed sample, can also range from, for example, 1 nanoliter to 500 microliters, such as from 1 nanoliter to 100 microliters, such as from 1 nanoliter to 10 microliters, such as from 1 microliter to 10 microliters. The volume of a sample, such as an unprocessed sample or a processed sample, can also range from, for example, 0.1 microliter to 100 microliters, such as from 5 microliters to 100 microliters, such as from 10 microliters to 100 microliters, or may range from 0.1 microliter to 10 microliters. The volume of a sample, such as an unprocessed sample or a processed sample, can also range from, for example, 1 picoliter to 10 microliters, such as from 1 picoliter to 5 microliters, such as from 1 picoliter to 2 microliters. All of such listed ranges are inclusive. The volume of a sample, such as an unprocessed sample or a processed sample, can also be, for example, 5000 microliters or less, such as 1000 microliters or less, such as 500 microliters or less, such as 200 microliters or less, such as 100 microliters or less, such as 50 microliters or less, such as 25 microliters or less, such as 10 microliters or less, such as 1 microliter or less. The volume of a sample, such as an unprocessed sample or a processed sample, can also be, for example, 1 microliter or greater, such as 10 microliters or greater, such as 25 microliters or greater, such as 50 microliters or greater, such as 100 microliters or greater, such as 200 microliters or greater, such as 500 microliters or greater, such as 1000 microliters or greater, such as 5000 microliters or greater. The volume of a sample, such as an unprocessed sample or a processed sample, can also be, for example, greater than 1 microliter, greater than 10 microliters, or greater than 100 microliters. The volume of a sample, such as an unprocessed sample or a processed sample, can also be, for example, less than 1 microliter, less than 10 microliters, or less than 100 microliters.

As described above, in some embodiments, a solution sample in which an analyte has not yet been concentrated, such as concentrated within a staging reservoir using electrophoresis as described herein, may be referred to as a sample, an initial sample, and/or an unprocessed sample. Also, a solution sample in which an analyte has been concentrated, such as concentrated within a staging reservoir using electrophoresis as described herein, may be referred to as a sample and/or a processed sample.

In various embodiments, a sample, e.g., an unprocessed sample, and/or a sample reservoir, such as a large volume reservoir, has a volume of, for example, 0.1 μL or greater, such as 1 μL or greater, such as 5 μL or greater, such as 10 μL or greater, such as 100 μL or greater, such as 1 mL or greater, such as 100 mL or greater, such as 1000 mL or greater, or 5000 mL or greater. In some embodiments, a sample, e.g., an unprocessed sample, and/or a sample reservoir, such as a large volume reservoir, has a volume of 100 μL or less, such as 10 μL or less, such as 1 μL or less. In some embodiments, a sample, e.g., an unprocessed sample, and/or a sample reservoir, such as a large volume reservoir, has a volume ranging, for example, from 0.01 μL to 5000 mL, 0.1 μL to 1000 mL, 1 μL to 50 mL, 1 μL to 1 mL, 1 μL to 500 μL, 1 μL to 100 μL, or 5 μL to 50 μL, with each of the listed ranges provided in this disclosure being inclusive, i.e., including, and not excluding, both of their terminal values. In various embodiments, a sample reservoir, such as a large volume reservoir, is configured to receive entirely therein a sample, e.g., an unprocessed sample. As such, in some aspects, a sample reservoir, such as a large volume reservoir, has a volume larger, such as slightly larger, than a volume of a sample, e.g., an unprocessed sample, received therein.

In various aspects, a staging reservoir, as described herein, is configured to receive entirely therein a sample, such as a processed sample. As such, in some aspects, a staging reservoir has a volume larger, such as slightly larger, than a volume of a sample, e.g., a processed sample, received therein. In some embodiments, a sample, e.g., a processed sample, and/or a staging reservoir, has a volume ranging, for example, from 0.01 μL to 100 mL, 0.01 μL to 1 mL, 0.01 μL to 1 mL, 0.1 μL to 500 μL, 0.1 μL to 100 μL, 1 μL to 50 μL, or 1 μL to 10 μL, each inclusive or from 1 nL to 5 μL, inclusive. Also, in various embodiments, a sample, e.g., a processed sample, and/or a staging reservoir, has a volume, for example, of 0.01 μL or greater, 0.1 μL or greater, such as 1 μL or greater, such as 5 μL or greater, such as 10 μL or greater, such as 50 μL or greater, such as 100 μL or greater, such as 500 μL or greater, such as 1 mL or greater, such as 10 mL or greater, such as 100 mL or greater. In some embodiments, a sample, e.g., a processed sample, and/or a staging reservoir, has a volume, for example, of 0.01 μL or less, 0.1 μL or less, 1 μL or less, 5 μL or less, 10 μL or less, 50 μL or less, 100 μL or less, 500 μL or less, 1 mL or less, 10 mL or less, or 100 mL or less.

In some embodiments, a sample, e.g., an unprocessed sample or a processed sample, and/or a reservoir, such as a sample reservoir, such as a large volume reservoir, or a staging reservoir, may have a volume in an inclusive range from 10 μl to 50 mL, such as from 20 μl to 20 mL, such as from 20 μl to 100 μl. A sample, e.g., an unprocessed sample or a processed sample, and/or a reservoir, such as a sample reservoir, such as a large volume reservoir, or a staging reservoir, may have a volume in an inclusive range from 100 μl to 200 μl, from 200 μl to 300 μl, from 300 μl to 400 μl, from 400 μl to 500 μl, from 500 μl to 600 μl, from 600 μl to 700 μl, from 700 μl to 800 μl, from 800 μl to 900 μl, from 900 μl to 1 mL, from 1 mL to 10 mL, from 10 mL to 25 mL, or from 25 mL to 50 mL. In some embodiments, a sample, e.g., an unprocessed sample or a processed sample, and/or a reservoir, such as a sample reservoir, such as a large volume reservoir, or a staging reservoir, has a volume of from 10 μl to 500 μl, or from 500 μl to 10 mL. In some embodiments, the sample, e.g., an unprocessed sample or a processed sample, and/or a reservoir, such as a sample reservoir, such as a large volume reservoir, or a staging reservoir, has a volume of 1 μl or greater, such as 10 μl or greater, such as 20 μl or greater, such as 50 μl or greater, such as 100 μl or greater, such as 500 μl or greater, such as 1 mL or greater.

In some embodiments of the disclosed embodiments, an unprocessed sample, and/or a sample reservoir, such as a large volume reservoir, has a volume which is larger, such as significantly larger, than a volume of a processed sample, and/or a staging reservoir. For example, in some aspects, a volume of an unprocessed sample, and/or a sample reservoir, such as a large volume reservoir, has a volume which is, for example, 2× (two times) or more, 3× or more, 5× or more, 10× or more, 20× or more, 50× or more, 100× or more, 500× or more, 1000× or more, 2000× or more, 5000× or more, or 10000× or more, than the volume of a processed sample, and/or a staging reservoir. In some embodiments, a volume of a processed sample, and/or a staging reservoir is smaller by (e.g., reduced by) a factor of, for example, 2× or more, 3× or more, 5× or more, 10× or more, 20× or more, 50× or more, 100× or more, 500× or more, 1000× or more, 2000× or more, 5000× or more, or 10000× or more, relative to a volume of an unprocessed sample, and/or a sample reservoir, such as a large volume reservoir. In some embodiments, a volume of a processed sample, and/or a staging reservoir is smaller than a volume of an unprocessed sample, and/or a sample reservoir, such as a large volume reservoir by a factor ranging, for example, from 1.5× to 5000×, 1.5× to 1000×, 2× to 5000×, 2× to 1000×, 2× to 500×, 2× to 100×, 2× to 50×, 2× to 10×, or 2× to 5×, each inclusive.

In some embodiments, an unprocessed sample, has a volume which is larger, such as considerably larger, than a volume of a processed sample. For example, an unprocessed sample may have a volume which is larger than a volume of a processed sample by 0.1 μL or more, such as 1 μL or more, such as 10 μL or more, such as 50 μL or more, such as 100 μL or more, such as 500 μL or more, such as 1 mL or more, such as 10 mL or more, such as 50 mL or more, such as 100 mL or more, such as 500 mL or more, such as 1000 mL or more, such as 2000 mL or more, such as 5000 mL or more. In some embodiments an unprocessed sample has a volume which is 2× (two times) or more, 3× or more, 5× or more, 10× or more, 20× or more, 50× or more, 100× or more, 500× or more, 1000× or more, 2000× or more, 5000× or more, or 10000× or more, than the volume of a processed sample. Accordingly, processing a sample, as is described herein, may reduce the sample's volume by a factor of 2× (two times) or more, 3× or more, 5× or more, 10× or more, 20× or more, 50× or more, 100× or more, 500× or more, 1000× or more, 2000× or more, 5000× or more, or 10000× or more. Also, processing a sample, may reduce the sample's volume by 0.1 μL or more, such as 1 μL or more, such as 10 μL or more, such as 50 μL or more, such as 100 μL or more, such as 500 μL or more, such as 1 mL or more, such as 10 mL or more, such as 50 mL or more, such as 100 mL or more, such as 500 mL or more, such as 1000 mL or more, such as 2000 mL or more, such as 5000 mL or more.

In some aspects, a sample reservoir, such as a large volume reservoir, has a volume which is larger, such as considerably larger than a volume of a staging reservoir. For example, a sample reservoir, such as a large volume reservoir, may have a volume which is larger than a volume of a staging reservoir by 0.1 μL or more, such as 1 μL or more, such as 10 μL or more, such as 50 μL or more, such as 100 μL or more, such as 500 μL or more, such as 1 mL or more, such as 10 mL or more, such as 50 mL or more, such as 100 mL or more, such as 500 mL or more, such as 1000 mL or more, such as 2000 mL or more, such as 5000 mL or more. In some embodiments a sample reservoir, such as a large volume reservoir, has a volume which is 2× (two times) or more, 3× or more, 5× or more, 10× or more, 20× or more, 50× or more, 100× or more, 500× or more, 1000× or more, 2000× or more, 5000× or more, or 10000× or more, the volume of a staging reservoir. Furthermore, as is described herein, a sample reservoir, such as a large volume reservoir, may be configured to receive therein, such as receive entirely therein, an unprocessed sample having any of the volumes provided in this disclosure. Also, a staging reservoir may be configured to receive therein, such as receive entirely therein, a processed sample having any of the volumes provided herein.

In accordance with the subject disclosure, the subject devices or portions thereof, e.g., an electrophoresis assembly, are configured to move an analyte via electrophoresis from a first portion to a second portion of a device. For example, an analyte may be electrophoretically transported within a device, e.g., transported directly to a staging reservoir from a sample reservoir or via a connector to a staging reservoir. As indicated above, electrophoresis refers to the movement of charged molecules or particles in solution in response to an electric field. The mobility of an analyte is based on (1) a net charge of the analyte molecule itself and/or (2) a net charge of an ionic moiety associated with the analyte, as described below. A net charge is the combination ionic charge that a molecule has, and it can be positive, negative, or neutral. As noted above, the analyte may be electrophoresed through an aqueous buffer solution with or without the use of size exclusion gels, a viscous medium, filters, biological sieves or the like.

In some embodiments, the devices or electrophoresis assemblies thereof include at least a first electrode, e.g., a microelectrode, and a second electrode, e.g., a microelectrode, which are configured so that an electric field may be generated between them. Generation of such an electric field may move an analyte between the electrodes, e.g., microelectrodes, such as from a sample reservoir to a staging reservoir, with the device.

The subject devices may include one or more connector, such as a tube, configured to convey one or more liquids, e.g., convey one or more liquids, such as a liquid solution, such as a buffer and/or analyte, from a first location to a second location. The one or more connector through which a solution, e.g., a buffer solution, and/or an analyte travels, such as the connectors 112, 113, 114, 115, in FIG. 1, such as fluidic connectors, can have any of a variety of forms and be made of any material compatible with the solution and/or analyte and which does not interfere with movement of the charged analyte and/or the electrophoresis. In some embodiments, a connector has multiple regions with different dimensions. A connector extending between a sample reservoir, e.g., a large volume reservoir, and a staging reservoir and/or a staging reservoir and an analysis element may have sections of increasing and/or decreasing of one or more dimensions, e.g., length, width, or diameter. A connector can be formed, for example, by connecting straight or tapered tubes or channels of decreasing diameters and of different materials. In some embodiments, a connector may include a microfluidic channel, e.g., a microfluidic channel of a microfluidic assembly, with the analyte then being transported via the channel to an analysis element. The channels can be manufactured to have various shapes and dimensions using, for example, elastomer molding, photolithography and/or micro-machining methods. In some embodiments a connector is entirely separate from an analysis assembly, such as a microfluidic assembly.

For the subject connectors, e.g., tubing, there are a wide variety of sizes and materials to select from. Examples of methods for forming these types of connectors can be found, for example, in Douglas Smith, Engineering and Science, published by California Institute of Technology, 2003 volume LXVI, Number 2, page 8-18; Skelley A M et al., Proc Natl Acad Sci USA. 2005 Jan. 25; 102(4):1041-6; Manz, A. and Becker, H., “Microsystem Technology in Chemistry and Life Sciences” published by Springer-Verlag, 1999. Exemplary connectors, e.g., tubes, include stainless steel tubes, needles, tubing made of plastic material such as polypropylene, polytetrafluoroethylene, Teflon, polyvinylchloride, PEEK™ or PEEKsil™, fused silica or glass. Suitable connectors, e.g., tubes, may have an inner diameter ranging from several millimeters to microns, including but not limited to 10 mm, 9 mm, 8 mm, 7 mm, 6 mm, 5 mm, 4 mm, 3 mm, 2 mm, 1 mm, 0.5 mm, 0.1 mm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, or 1 μm. In some embodiments, the inner dimensions of a connector are in the range of 5 mm to 50 μm.

A staging reservoir according to the subject embodiments may have a variety of capacities and/or volumes. For example, a staging reservoir may have a capacity of, for example, from 1 picoliter to 2 microliters, including, but not limited to 1 picoliter to 1 microliter, and 10 picoliters to 0.5 microliter. A staging reservoir may also have a capacity ranging from, for example, 0.1 microliter to 10 mL, such as from 0.1 microliter to 1 mL, such as from 0.1 microliter to 10 microliters. A staging reservoir may also have a capacity ranging from, for example, 1 microliter to 10 mL, such as from 10 microliters to 10 mL or 5 microliters to 10 mL. A staging reservoir may also have a capacity ranging from, for example, 100 microliters to 2 mL. A staging reservoir may also have a capacity ranging from, for example, 1 nanoliter to 500 microliters, such as from 1 nanoliter to 100 microliters, such as from 1 nanoliter to 10 microliters, such as from 1 microliter to 10 microliters. A staging reservoir may also have a capacity ranging from, for example, 0.1 microliter to 100 microliters, such as from 5 microliters to 100 microliters, such as from 10 microliters to 100 microliters, or may range from 0.1 microliter to 10 microliters. A staging reservoir may also have a capacity ranging from, for example, 0.1 picoliter to 10 microliters, such as from 1 picoliter to 5 microliters, such as from 1 picoliter to 2 microliters. All of such listed ranges are inclusive. A staging reservoir may also have a capacity which is, for example, 5000 microliters or less, such as 1000 microliters or less, such as 500 microliters or less, such as 200 microliters or less, such as 100 microliters or less, such as 50 microliters or less, such as 25 microliters or less, such as 10 microliters or less, such as 1 microliter or less. A staging reservoir may also have a capacity which is, for example, 1 microliter or greater, such as 10 microliters or greater, such as 25 microliters or greater, such as 50 microliters or greater, such as 100 microliters or greater, such as 200 microliters or greater, such as 500 microliters or greater, such as 1000 microliters or greater, such as 5000 microliters or greater. A staging reservoir may also have a capacity which is, for example, greater than 1 microliter, greater than 10 microliters, or greater than 100 microliters. A staging reservoir may also have a capacity which is, for example, less than 1 microliter, less than 10 microliters, or less than 100 microliters.

Also, in one orientation of an actuator, a staging reservoir may be in operable, e.g., fluidic, communication with an analysis element of a device. In such an embodiment, an analyte can be transported from the staging reservoir to the analysis element when, for example, appropriate gates and/or valves are open and/or closed. As is described throughout this specification, the ratio of the volumes in a sample reservoir, e.g., large volume reservoir, and the staging reservoir can vary considerably, but in some embodiments, the ratios of the respective reservoirs are from 100:1 to 1000:1, including but not limited to, 100:1 or more, 200:1 or more, 300:1 or more, 500:1 or more, 800:1 or more, and 1000:1 or more, wherein the staging reservoir's volume is smaller than the sample reservoir's volume in each ratio.

To generate an electric field for transportation of an analyte from a sample reservoir, e.g., large volume reservoir, to a staging reservoir, electrodes, such as microelectrodes, may be situated to generate such a field. In some embodiments, electrodes are situated within each of the reservoirs, e.g., the sample reservoir and/or the staging reservoir, and/or within a first buffer reservoir, such as a low salt buffer reservoir, and a second buffer reservoir, such as a high salt buffer reservoir, of an electrophoresis apparatus. Electrodes may also be situated on first and second sides of the actuator and/or analysis element within an analysis assembly, such as a microfluidic assembly. The location of the electrode in the sample reservoir may vary, but may be a distance from an opening of a connector, such as at the furthest site or distance from an opening fluidically connecting the sample reservoir to the staging reservoir. The electrodes can be attached or positioned in a way that allows an electric field for electrophoresis to be generated when the current is on and solution is present to complete a circuit. For example and not limitation, electrodes can be inserted into the liquid in a reservoir. Also, an electrode can be integral to a device or one or more portions thereof, such as an electrophoresis assembly and/or an analysis assembly, such as a microfluidic assembly, e.g., incorporated into the wall of a reservoir or chamber. For more information of microelectrodes which may be applied with the subject embodiments, see, e.g., International Patent Publication WO04044575A2; Rongsheng et al., 2005, Anal. Chem 77:4338-47; and Abad-Villar et al, 2005, Electrophoresis 26:3602-3608, the disclosures of which are incorporated herein in their entireties and for all purposes.

In addition, the polarity of one or more electrodes in the device or system can be changed, e.g., changed from a negative to a positive charge, such as changed via a power supply controller, one or more times during the operation of the device. A charged analyte migrates away from one or more electrodes of like charge and towards one or more electrodes of opposite charge. Thus, for example, a positively charged analyte can be transported by electrophoresis from a reservoir, e.g., an LVR, having a positively charged electrode (anode) to a staging reservoir, such as a staging reservoir having a negatively charged electrode (cathode). The analyte can then be, for example, transported from the staging reservoir to an analysis element by turning off current to the electrodes in the electrophoresis assembly, actuating the actuator, and turning on electrodes positioned in the analysis assembly, such that the analyte is transported through or across the analysis element. The rate of migration depends on the strength of the field, the net charge, the size and shape of the molecules and also on the ionic strength, viscosity and temperature of the medium in which the molecules are moving.

Reagents used for capture and analysis of the analyte may be prepositioned in channels of the device or portions thereof, such as the analysis assembly, such as the microfluidic assembly, e.g., capture agents immobilized on the analysis elements substrate, may be introduced from the staging reservoir along with the analyte, and/or may be introduced on the analysis assembly, such as the microfluidic assembly. For example, reagents can be introduced into the staging reservoir, sample reservoir, analysis element, and/or channels by various methods at any point appropriate for the assay. Methods for introduction will vary with the specific design of the device and the conditions of the assay. For illustration, reagents may be introduced into the analysis element via an input channel in fluidic communication with the analysis element by opening a valve that separates the input channel and analysis element.

B. Systems

Embodiments of the subject disclosure include systems, such as systems including one or more, such as two, three, four, five or more, ten or more, or twenty or more, devices. In some embodiments, one or more of the devices of a system include one or more components, such as an analysis element, for detecting an analyte or one or more characteristics thereof.

In some embodiments of the systems, the systems include a first device and a second device. In some embodiments, a first device and/or a second device of a system is any of the devices described herein and/or may include any element described herein, or combination thereof. For example, in some embodiments, a first device may include an electrophoresis assembly including a sample reservoir. A first device may also include an actuator including a staging reservoir and which is actuable between a first configuration and a second configuration. In some embodiments of such a device, the staging reservoir is operably connected with the sample reservoir when the actuator is in the first configuration and the staging reservoir is accessible to extract a sample therefrom when the actuator is in a second configuration. In other words, in a second configuration, and/or in an additional configuration, a staging reservoir may be operably connected or operably connectable to a conduit or container for transferring a processed sample away from the staging reservoir. Such a conduit or container may be a beaker, a tube, and/or another container type and may be used to transfer the processed sample to a second location, such as within a second device and/or into a storage area for storage of the processed sample. Also, transferring a processed sample away from the staging reservoir may be automatic, such as controlled by one or more controller, such as a computer controller and/or manual. Furthermore, the subject methods include in some embodiments, using the systems. For example, the methods may include automatically and/or manually transferring a processed sample from a first device to a second or additional device or to a sample storage container and/or area configured for sample storage.

The systems of the subject disclosure may also include a second device, such as a detection device. Such a second device may include one or more elements configured for analyzing a sample, such as a processed sample, such as an analysis element. A second device may also include an analysis assembly or one or more components thereof.

Devices of the subject systems, such as a first device and an additional device, such as a second device, may be operably connectable and may be separate and/or independently functional devices. Devices of the subject systems may each be substantially contained within their own respective housing and/or may include one or more elements that extend outside of the housing for forming one or more operable connection, such as a fluidic and/or electrical connection. In some embodiments, devices of the systems are separate, unattached, and/or independently functional from one another. In some embodiments, devices of the systems are configured to be removably coupled to one another to form a reversibly operable connection. In some embodiments, devices of the systems are integral with one another, such as both contained within a single housing.

C. Methods

Methods for operation of the subject devices and systems are included herein. A method of operation of an embodiment of the device is shown in FIGS. 4A and 4B. In FIGS. 4A and 4B, the depicted apparatus includes a two-way valve with an actuator 401 and a channel, such as a staging reservoir 405, which, in a first position, such as a vertical position, connects a first reservoir 402, such as an upper reservoir, e.g., a sample reservoir, and a second reservoir 403, such as a lower reservoir, of the electrophoresis device 400. While directional terms such as “upper” and “lower” are used for convenience with reference to the figures, it should be noted that no particular directionality is required. Thus, for example, the reservoirs could equally be described as “left” and “right” reservoirs or vice versa. Charged analyte particles, such as molecules, in a large volume are placed in the first reservoir 402, which may be a low salt buffer reservoir, and a voltage is applied so that the molecules migrate toward the second reservoir 403, which may be a high salt buffer reservoir. In some embodiments, a sample, such as an unprocessed sample is placed within a first reservoir 402, or a portion thereof, which may be a sample reservoir, e.g., a large volume reservoir. Then a buffer, such as a low salt buffer, is layered on top of the sample in the first reservoir 402. Alternatively, a buffer, such as a low salt buffer, is placed in the first reservoir 402, or one or more portions thereof, which may include a sample reservoir, e.g., a large volume reservoir. Then, in a second step, a sample, such as an unprocessed sample is placed within the first reservoir 402, or a portion thereof, such as a sample reservoir, e.g., a large volume reservoir. After the buffer and unprocessed samples are added, a voltage is applied so that molecules, such as analyte molecules of the sample, migrate toward the second reservoir 403, such as a high salt reservoir. Positioned just below an end, such as a lower end, of the valve's channel, such as staging reservoir 405, is a filtering element 404, such as a membrane, such as a semi-permeable membrane. In some embodiments, a filtering element prevents and/or substantially inhibits one or more analyte of interest from flowing therethrough but allows other substances, e.g., a solution in which an analyte is suspended such as a buffer solution, therethrough. In some embodiments, the subject membranes include a plurality of pores which in turn allow the membranes to have a characteristic porosity and/or permeability. Furthermore, in some embodiments, the filtering element's, e.g., membrane's, porosity is such that it permits the passage of small ions and molecules but prevents the passage of the larger analyte molecules. In other words, a membrane according to the subject embodiments may be configured to inhibit movement of an analyte through the membrane. Thus, when a voltage is applied for a suitable length of time between the first reservoir 402, and the second reservoir 403, analyte molecules will migrate, e.g., migrate in a downward direction (in the context of FIG. 4), and concentrate on or immediately upstream of the filtering element 404 and within the two-way valve channel, such as staging reservoir 405. At this point, a voltage can be optionally applied in the reverse direction for a brief period of time to release concentrated analyte molecules from the filtering element 404, e.g., membrane, and move them into the valve's channel, such as staging reservoir 405. In other words, in some embodiments, the methods include reversing the movement, e.g., flow, of analyte molecules within the electrophoretic assembly to remove such molecules from the filtering element and/or further concentrate the molecules in the staging reservoir and/or remove or substantially remove the molecules from a connector between a filtering element and a staging reservoir. Also, the subject methods include processing a sample and thereby converting it from an unprocessed sample to a processed sample by concentrating an analyte in the sample within or near the staging reservoir 405.

After analyte molecules are concentrated and located within the valve's channel, such as a staging reservoir 405, the valve with the actuator 401 can be turned to a second, e.g., horizontal, position causing the valve's channel to be operably connected, e.g., fluidically connected, to and/or in line with a fluidic channel 406, such as a microfluidic channel, of an analysis assembly, such as a microfluidic assembly and an analysis element or detection area 407 thereof and/or to a detection device and an analysis element or detection area 407 thereof. The sample including the concentrated analyte molecules can then be propelled, e.g., pumped, into the analysis element and analyte detection and/or analysis conducted thereon.

Also, after analyte molecules are concentrated and located within the valve's channel, such as a staging reservoir 405, the valve with the actuator 401 can be turned to a second, e.g., horizontal, position causing the valve's channel to be operably connected, e.g., fluidically connected, to an analysis element and/or a detection device or a portion thereof, such as an input port or an analysis element. In some embodiments, actuating the actuator to a second position allows the sample to be accessed and transferred to a transferring element, such as a tube or pipette. Accordingly, the methods may include transferring, such as manually and/or automatically transferring, a sample from the sample reservoir to a detection device and/or an analysis element. Such a detection device and/or analysis element may be integral with or separate and independent from the device including the electrophoresis assembly. Also, in some embodiments, the methods include transferring a sample from the sample reservoir to a storage and storing the sample for a period of time, e.g., 1 min or less, 1 hour or less, 1 day or less, 1 week or less, 1 month or less, 6 months or less, 1 year or less, 5 years or less, or more than 5 years.

In addition, as noted above, analysis elements may be configured for detecting an analyte and/or analyzing properties of sample, such as one or more characteristics of an analyte or a component in a sample. Analysis elements may be independently operating instruments and may be configured to detect an analyte or analyze properties of a sample by performing one or more assays including, for example: well-plate based assays, enzyme-linked immunosorbent assay (ELISA), immunoassays, colorimetric assays, phosphogenic assays, luminogenic assays, fluorimetric assays, radionuclide-based assays, Nuclear magnetic resonance (NMR), mass spectrometry (MS), a type of spectrometry, capillary electrophoresis (CE), capillary zone electrophoresis (CZE), Polyacrylamide gel electrophoresis (PAGE), gel electrophoresis, biosensor-based device assays, fluidic device assays including lab-on-chip, lateral flow assays, passive flow assays, chromatography, quantitative polymerase chain reaction (qPCR), polynucleotide sequencing, e.g., DNA sequencing or RNA sequencing, or any combinations thereof. Accordingly, the subject methods include performing any one or combination of such assays to analyze a sample and/or detect an analyte or a characteristic thereof.

According to various embodiments of the methods, the subject apparatus or portions thereof, such as an electrophoresis assembly and/or an actuator, can be inserted in a fluidic, e.g., microfluidic, path of any fluidic detection device, such as a fluidic analyte detection device, and does not require any modification of the detection device itself. Furthermore, as is disclosed herein, the dimensions of the electrophoresis apparatus reservoirs, such as an upper reservoir and/or lower reservoir and/or sample reservoir, as well as the dimensions and volume of the valve's channel, e.g., staging reservoir, can be varied so that a first reservoir, such as an upper and/or sample, reservoir is suitable for the intended initial large sample volume, and the valve's channel, volume approximates that of a detection chamber and/or analysis element of the fluidic detection device in order to maximize analyte capture on a detection surface thereof. In some embodiments, the volume of the staging reservoir is equal to or larger than the detection chamber volume. It is also notable that according to various embodiments of the methods, electrophoresis may be performed horizontally and the reservoirs, such as the first and second buffer reservoirs, are to the left and right of the valve's channel, e.g., staging reservoir.

In some embodiments, the disclosed methods include introducing an analyte in a sample to an analysis assembly, such as a microfluidic assembly. Such methods may include electrophoresing by applying an electric potential to a sample including an analyte into a staging reservoir of an actuator. Applying such an electric current may include generating an electric field between two electrodes. Electrophoresis according to the subject embodiments may include applying an electrophoresis assembly, as such assemblies are described herein to move the analyte within the device. The analyte can be moved within the device, for example, by electrophoresing the analyte from the sample reservoir, e.g., large volume reservoir, to the staging reservoir. In some embodiments, the sample is moved from the sample reservoir, e.g., large volume reservoir, to the staging reservoir via connector, such as a conduit, such as a tube. In some embodiments, the staging reservoir has one or more characteristics of the large volume reservoirs described herein.

The methods also may include concentrating the analyte in the staging reservoir. Concentrating an analyte may be achieved by applying an electric field across a membrane or other filtering element which restricts flow of analyte therethough. Such an electric field may be generated between electrodes, e.g., microelectrodes on respective sides of the membrane.

The actuator can, in some aspects of the methods, be moved, e.g., rotated, to operably connect the staging reservoir to an analysis element of the analysis assembly, such as the microfluidic assembly. In some embodiments, the actuator includes a valve and actuating the actuator includes rotating the valve, e.g., rotating the valve 90°, from a first configuration to second configuration.

The methods, in some embodiments, include a step of propelling the sample into the analysis element of an analysis assembly, such as the microfluidic assembly and/or performing analysis on the sample. Such propelling may be achieved by applying an electric field between electrodes such as, for example, a first microelectrode at a first end of the analysis assembly, such as the microfluidic assembly, and a second microelectrode at a second end of the assembly. Propelling may also be achieved by applying a pressure, such as a fluidic pressure, to a sample using, for example, a pump.

As noted above, subject methods include applying microfluidic devices and assemblies as described herein. A number of methods and approaches are available for making microfluidic devices. Such approaches include microassembly, bulk micromachining methods, surface micro-machining methods, standard lithographic methods, wet etching, reactive ion etching, plasma etching, stereolithography and laser chemical three-dimensional writing methods, soft lithography methods, modular assembly methods, replica molding methods, injection molding methods, hot molding methods, laser ablation methods, combinations of methods, three-dimensional (3D) printing, and other methods. For general reviews providing further details of microfluidic devices and assemblies see, for example, the following references which are incorporated by reference herein in their entireties and for all purposes: Chovan, et al. “Microfabricated devices in biotechnology and biochemical processing” Trends Biotechnol. 2002 20:116-22; Anthony et al. “DNA array technology and diagnostic microbiology” Expert Rev. Mol. Diagn. 2001 1:30-8; Windman et al. “Microfluidics for ultrasmall-volume biological analysis” Adv. Chromatogr. 2003, 42:241-67; and Ng et al. “Biochips beyond DNA: technologies and applications” Biotechnol Annu Rev. 2003, 9:1-149; Fiorini and Chiu, 2005, “Disposable microfluidic devices: fabrication, function, and application” Biotechniques 38:429-46; Beebe et al., 2000, “Microfluidic tectonics: a comprehensive construction platform for microfluidic systems.” Proc. Natl. Acad. Sci. USA 97:13488-13493; Rossier et al., 2002, “Plasma etched polymer microelectrochemical systems” Lab Chip 2:145-150; Becker et al., 2002, “Polymer microfluidic devices” Talanta 56:267-287; Becker et al., 2000, “Polymer microfabrication methods for microfluidic analytical applications” Electrophoresis 21:12-26; U.S. Pat. No. 6,767,706 B2, e.g., Section 6.8 “Microfabrication of a Silicon Device”; Terry et al., 1979, A Gas Chromatography Air Analyzer Fabricated on a Silicon Wafer, IEEE Trans, on Electron Devices, v. ED-26, pp. 1880-1886; Berg et al., 1994, Micro Total Analysis Systems, New York, Kluwer; Webster et al., 1996, Monolithic Capillary Gel Electrophoresis Stage with On-Chip Detector in International Conference On Micro Electromechanical Systems, MEMS 96, pp. 491496; Unger et al., 2000, Science 288:113-16; U.S. Pat. No. 6,960,437 (Nucleic acid amplification utilizing microfluidic devices); Quake & Scherer, 2000, “From micro to nanofabrication with soft materials” Science 290:1536-40; Becker et al., 2000, “Polymer microfabrication methods for microfluidic analytical applications” Electrophoresis 21:12-26. Also described are electrodes, such as microelectrodes, suited for use in analysis assemblies, such as microfluidic assemblies and devices. Also described are methods for immobilizing one or more analyte, such as proteins, nucleic acids or other molecules, on a surface of the device, e.g., within a microfluidic channel.

Microfluidic devices (sometimes referred to as “chips”), such as those disclosed herein, can be used, for example, in a variety of biomedical and pharmaceutical applications, including analysis, preparation and synthesis of chemical compounds and analysis and manipulation of cells, proteins and nucleic acids. The advantages of the miniaturization generally offered by microfluidic devices include greatly reduced consumption of reagents, shorter reaction times, and the potential of very high throughput using massively parallel-testing. However, one aspect of this miniaturization also may be a significant obstacle that limits the sensitivity of these procedures. Microfluidic chips may, in some cases, handle only minute volumes of sample solutions, and there may be insufficient analyte molecules in a very small volume applied to the chip to be readily detected. Thus, an amplification step, such as PCR, may be used to treat the sample for use with a microfluidic device, either before or after introduction of the sample to the device. However, this has the disadvantage of increasing the cost and complexity, and allowing possible aberrant results due to PCR mistakes. Some microfluidic devices are configured to receive a somewhat larger sample volume by exposing the entire surface of the chip to the incoming sample solution. This allows a relatively large sample volume to be applied since the whole chip surface is used to receive the sample, but still limits the volume of the sample that can be processed. Moreover, this limits the detection to one sample at a time and requires different sample solutions to be tested sequentially. For testing multiple samples, this is not only time-consuming, but has the additional disadvantage of risking sample-to-sample cross-contamination.

The methods and apparatus disclosed herein may be applied to overcome the limitation of low volume capacity on microfluidic devices, for example, by using electrophoresis to concentrate and/or control the transport of the analyte material, so that it can be applied to facilitate reactions or analysis and/or used in a typical chip format. In some embodiments, an analyte is moved, e.g., electrophoresed, through an aqueous buffer solution, such as a buffer solution within an electrophoresis assembly and/or an analysis assembly, such as a microfluidic assembly and/or an actuator, with or without the use of size exclusion gels, a viscous medium, filters, biological sieves or the like. The method and associated devices allow multiple samples to be applied to multiple specific areas on an analysis element of, for example, a chip. In some embodiments, because each sample may be separately electrophoresed to a different part of a chip for analysis, separate samples do not come in contact with the same surface. Such an embodiment has the advantage of reducing cross-contamination.

The subject disclosure provides methods and devices for analysis, such as microfluidic analysis, of one or more analytes from, for example, a large volume sample. Because the sample volume capacity of a device may be increased by the methods disclosed herein, the minimal detectable concentration, which is the lowest analyte concentration the assay can reliably measure, for an assay using the device is reduced.

The methods and devices described herein allow an investigator or clinician to extract analyte from a large volume sample, detect and identify the analyte or one or more characteristics thereof. The methods are suitable for use with a wide variety of analytes, including specific proteins and polynucleotide sequences. In addition to the ability to analyze multiple samples on a single chip simultaneously, embodiments of the methods allow for concentration of a large sample onto the chip without continuously flowing the sample into the concentration reservoir or onto the chip.

### II. The Analyte

According to embodiments of the subject disclosure, the analyte (or “analyte of interest”) is a molecule, complex of molecules or particle that may be measured and/or detected using the methods and devices disclosed herein. As noted above, an analyte has a net charge and/or can be associated with a charged molecule, so that the analyte can be electrophoretically concentrated as described herein. In some embodiments the analyte is associated with one or more ionic moieties, which carry a charge. Alternatively, the analyte can be associated with a charged molecule by binding to a charged carrier molecule, such as an antibody, a receptor, a ligand, a nucleic acid, a substrate, or an antigen. The carrier molecule can be intrinsically charged or can be modified to be charged by attaching an ionic moiety.

Examples of analytes include, but are not limited to, proteins, protein complexes, viruses, nucleic acids, heavy metals, drugs, steroids, and pesticides, and carbohydrates. In some embodiments, the analytes are biomolecules, i.e., a class of molecules that are produced in or by a cell, such as proteins, peptides, polynucleotides (e.g., RNA or DNA), sugars, lipids, glycolipids, glycoproteins, and the like. Particular examples of analytes include biomolecules from pathogenic organisms such as viruses or bacteria, biomolecules associated with disease, toxins, drugs, small molecules, prions, nucleic acids containing mutations, antibodies, and antigens. Analytes that may be analyzed using the methods of the subject disclosure may or may not have a net charge under the conditions, e.g., pH, of the assay. A polynucleotide is an example of an analyte that is itself charged. An analyte, whether charged or not, can be modified by attachment of at least one ionic moiety, to increase its charge. For example, an analyte can be modified by attachment of a carrier molecule that carries an ionic moiety, for example, an antibody that carries a nucleic acid ionic moiety. Examples of ionic moieties are described below.

### III. The Sample Solution and Pre-Sample

As used herein, the “sample solution” is the analyte-containing aqueous liquid that is present in the device at the start of electrophoresis (i.e., the “starting material”). In general, the sample solution is generated by processing a “pre-sample” that contains the analyte. Such processing is carried out to, for example, partially purify or concentrate the analyte, remove impurities that would interfere with electrophoresis or the assay, and the like. Examples of specific processing steps include centrifugation to remove debris, or to fractionate the pre-sample, precipitation, filtration, chromatography, sonication, or any other process that results in analyte free in solution. In some embodiments, processing includes concentration of the analyte using beads, such as magnetic beads, treated to bind the analyte. In addition, reagents may be added to the sample solution to adjust the pH, ionic strength and/or composition of the solution to facilitate electrophoresis by, for example, adding buffering agents, acids, bases or salts. Addition may entail, for example, diluting the analyte-containing liquid with an appropriate solution such as water or buffer, such as a buffered salt solution, re-suspending the analyte in an appropriate solution, dissolving solids, e.g., salts, in the solution, and the like. In addition, the analyte in the sample solution may be modified by being associated with one or more ionic moieties, and optionally one or more carrier molecules.

The source of analyte can be any of a wide variety of materials, including for example, a biological fluid, cell, or tissue, environmental sample, e.g., a soil and/or water sample, or a synthetic product. Examples of biological pre-samples include, for example, blood, plasma, cerebro-spinal fluid, urine, saliva, cell extracts, tissue extracts, tissue culture extracts, cell extracts, cheek scrapings, and bacterial or viral cultures. Other examples of pre-sample includes lake or river water, food processing fluids, manufactured food preparations, fruit and vegetable extracts, and cosmetics. The pre-sample can be a liquid or a solid. If a solid, the pre-sample may be dissolved, solubilized and/or suspended in a liquid, e.g., aqueous liquid, and insoluble materials may be removed. Table 9 shows, for illustration and not limitation, exemplary samples and pre-samples.

TABLE 9
Exemplary Samples and Pre-samples

| Pre-sample | Sample | Analyte |
|---|---|---|
| blood | serum* | anti-HIV antibody** |
| urine | filtered urine* | hCG** |
| river water | filtered water* | cholera bacteria antigens** |
| PBMC | genomic DNA | DNA fragment** |

*In each case, optionally modified to adjust ionic strength/pH
**In each case, optionally modified to associate with an ionic moiety.

### IV. Ionic Moieties

An ionic moiety is a molecular structure that carries a charge. The ionic moiety can be anionic, such as polyanionic, or cationic, such as polycationic. It will be appreciated that the net charge of a charged molecule will depend in part on the environment, particularly the pH and salt composition of the sample solution. However, in some embodiments, the ionic moiety has a net charge of +5 or more or at least −5 or less. Although the ionic moiety may have a low or medium charge density, in some embodiments, the ionic moiety has a high charge density. Charge density is the amount of charge/per unit volume of a solution, material, and the like due to the presence of charged entities within the material. A material having a high charge density has more charge per unit volume, and is more likely to attract entities having an opposite charge, and repel entities having the same charge. Typically, having a higher charge density will result in faster migration times of charged entities during electrophoresis.

Examples of ionic moieties include, for illustration and not for limitation, nucleic acids and their natural and synthetic analogs, e.g., RNA, DNA, PNA, poly-amines such as poly-lysine, poly-glutamate, poly-aspartate, sulfated glycans and chemically modified proteins such as succinylated bovine serum albumin. Other ionic moieties include polyacrylic acid, polymethacrylic acid, polyethylacrylic acid, polypropylacrylic acid, polybutylacrylic acid, polymaleic acid, dextran sulfate, heparin, hyaluronic acid, polysulfates, polysulfonates, polyvinyl phosphoric acid, polyvinyl phosphonic acid, copolymers of polymaleic acid, polyhydroxybutyric acid and mixed polymers.

### V. Association of the Analyte with an Ionic Moiety

Prior to electrophoresis, an analyte can be modified to be associated with an ionic moiety. The combination of the analyte and ionic moiety may have a net charge greater than that of the analyte alone. The analyte can be modified directly with the ionic moiety or indirectly using a carrier molecule that carries an ionic moiety. Carrier molecules include analyte-binding antibodies, polynucleotides and other molecules, as discussed below.

A. Direct Association of an Ionic Moiety and Analyte

In some embodiments, an ionic moiety is associated directly, either covalently or noncovalently, with an analyte. For example, a nucleic acid ionic moiety may be non-covalently associated with a nucleic acid analyte based on sequence complementarity (partial or complete). The analyte can also be covalently modified to increase its ionic charge either chemically or enzymatically. Examples of chemical modifications include converting amino groups in a protein to carboxyl groups to increase the net negative charge of the protein using reagents such as anhydrides, e.g., succinic anhydride or tetrahydrylphthalic anhydride. Other groups in a protein such as thiol or histidyl groups can also be converted to negatively charged groups such as carboxyl groups using reagents such as iodoacetate. In addition, these functional groups can be converted to a number of other active groups to facilitate the association of ionic moieties. Details of these and other modification reagents and modification methods are provided by: “Chemical Modification of Proteins” by Gary E. Means and Robert E. Feeney; “Bioconjugate Techniques” by Greg T. Hermanson; and “Chemical Reagents for Protein Modification” by Roger L. Lundblad), the disclosures of which are incorporated by reference herein in their entireties and for all purposes.

Certain ionic moieties, such as nucleic acids, poly-lysine, poly-arginine, poly-glutamate, poly-aspartate and sulfated glycans, have functional groups (such as amino or carboxyl groups) that can facilitate covalent association with the analyte. Moreover, ionic moieties can be derivatized by design to have desirable functional groups for conjugation with the analytes.

In addition, a number of commercially available cross-linking agents exist which can be used to attach carrier molecules and ionic moieties, e.g., homo-bifunctional reagents that will cross-link amino-to-amino or sulfhydryl-to-sulfhydryl groups; hetero-bifunctional reagents that will cross-link the amino-to-sulfhydryl groups, and the like. Selection of these and other cross-linking methods can be based on the specific requirements of the assay.

B. Association of an Ionic Moiety and Analyte Indirectly via a Carrier Molecule or Carrier Complex

An analyte also can be associated with an ionic moiety indirectly, via a carrier molecule or carrier complex. A carrier molecule is a molecule that specifically binds the analyte. Thus, the analyte and carrier molecule together constitute a “specific binding pair” or carrier complex. In this embodiment, the ionic moiety is linked or conjugated to the carrier molecule instead of, or in addition to, the analyte. Examples of binding pairs include but are not limited to, antibody-antigen pairs, receptor-ligand pairs, and other ligand:anti-ligand complexes. Typically the carrier molecule is an antibody that specifically binds the analyte.

TABLE 10
Exemplary Specific Binding Pairs

| Analyte | Carrier Molecule |
|---|---|
| antigen (e.g., protein) | antibody |
| antibody | antigen |
| polynucleotide strand | complementary polynucleotide strand |
| ligand (e.g., hormone) | receptor (e.g., hormone receptor) |
| immunoglobulin | Protein A |
| enzyme | enzyme cofactor or substrate |
| carbohydrate | lectin |

A carrier molecule can be associated with an ionic moiety using any of a variety of methods for associating molecules, some of which are discussed above. The selected methods will depend in part on the nature of the analyte, ionic moiety and carrier molecule. For example, one or more ionic moieties can be attached to carrier molecules using standard chemistry. For example, ionic moieties such as nucleic acids, poly-lysine, poly-arginine, poly-glutamate, poly-aspartate and sulfated glycans have functional groups (such as amino or carboxyl) that facilitate cross-linking to antibodies or other carrier molecules, or can be derivatized to carry such functional groups. Some potential carrier molecules, such as nucleic acids and proteins have functional groups (such as amino [including, for example, lysine and/or arginine], carboxyl [including, for example, aspartic acid and/or glutamic acid] or sulfhydryl [including, for example, cysteine]) that facilitate cross-linking to ionic moieties, or can be derivatized to carry such functional groups. The carrier molecules can be associated with the ionic moiety or moieties using various bi-functional linkers. Carrier molecules can be chemically modified to have specific functional groups for cross linking. For example, the sulfated glycans can be oxidized to have aldehyde functional groups, which can be used to react with amino groups. Oligonucleotides can be synthesized with a sulfhydryl or a primary amino group on one end. With the amino or sulfhydryl functional groups, the oligonucleotide can be cross-linked to the amino or sulfhydryl groups on the antibody molecules using available cross-linking reagents.

An ionic moiety can also be associated with a carrier molecule (and thus an analyte) indirectly, via one or more intermediate molecule. For example, if the analyte is an antigen, “AntigenA,” it can be indirectly associated with an ionic moiety by, for example, binding AntigenA by an anti-AntigenA monoclonal antibody (AAA-mAb) (“carrier molecule”) and binding the AAA-mAb with an ionically-labeled second antibody (anti-AAA-mAb). It will be recognized that this type of second-antibody type labeling is routine in immunoassays. The complex of molecules including the analyte and ionic moiety (in this example, AntigenA+AAA-mAb+anti-AAA-mAb-ionic moiety) can be referred to as a “carrier complex.” Although antibody-antigen associations are described in this example, the method is not limited to antibodies. Any specific binding pair in which one of the partners is the analyte may be used.

A carrier molecule and ionic moiety also can be associated via a specific binding pair where one or both members of the specific binding pair is conjugated to a tag. For example, a carrier molecule can be biotinylated and labeled using an ionic moiety conjugated to avidin. The tag is either avidin or biotin and allows binding of the ionic moiety via the tag at any step in the process. In another embodiment, an antibody carrier molecule is associated with an ionic moiety, such as a nucleic acid, as follows: A charged avidin molecule is prepared by adding 1, 2, or 3 biotinylated oligonucleotides to its biotin binding sites, leaving at least one of its four biotin sites unoccupied. A biotinylated carrier molecule (e.g., anti-analyte antibody) is bound to the avidin-biotin-oligonucleotide complex. Other examples of tags include without limitation poly-histidine tag, Glutathione tag, digoxin, and fluorescein. Antibodies with specific affinity for the tags are available for purchase or can be produced as needed.

Any of the methods described above in the context of ionically labeling a carrier molecule, can be used to ionically label a protein, nucleic acid, or other component of the carrier complex.

A molecule that is bound directly, e.g., covalently, to an ionic moiety can be referred to as “ionically labeled.” The complex of the analyte, associated ionic moiety(s), carrier molecule(s), if present, and/or any other molecules used to associate the ionic moiety(s) and analyte can be referred to as the “Analyte-Ionic Moiety (IM) complex.”

In addition, the association of the analyte and ionic moieties is sufficiently stable under the conditions of concentrative electrophoresis and, optionally, subsequent concentration analytical steps that the two remain associated as the assay is conducted.

### VI. Association of an Ionic Moiety with an Immobilized Analyte

Specific embodiments, which utilize the concentrative electrophoresis technology described above in connection with an ionic moiety associated-analyte are described below in more detail. However, it should be noted that applicable steps of the methods described below can also be used in connection with a charged analyte which is not associated with an ionic moiety. According to various aspects of the methods, the analyte can be:

a) bound to a solid or immobilized phase

b) associated with an ionic moiety

c) released from the solid or immobilized phase

d) concentrated in a staging reservoir using electrophoresis

e) electrophoresed or pumped to the analysis assembly and/or other microfluidic device

f) bound or detected in an analysis element using a capture agent that specifically recognizes
i) the analyte or
ii) the ionic moiety.
   Steps (a) and (b) can take place in either order and Steps (b) and (c) can take place in either order, provided (c) occurs after (a). For example, the order can be a→b→c; b→a→c; or a→c→b. Steps (a)-(c) are described below, for illustration and not for limitation. This method includes two independent specific analyte concentration steps and provides a highly sensitive assay method.

a. Analyte is Bound to a Solid or Immobilized Phase

The analyte may be bound to a solid or immobilized phase (used interchangeably herein) in any conventional way, by treating the sample solution with a specific binding partner (SBP) of the analyte immobilized on a solid phase. The solid phase may be, for illustration and not limitation, a surface of a sample reservoir, e.g., a large volume reservoir, a surface of a microtiter plate well, or a surface of a microparticle. In some embodiments, microparticles are used. In some embodiments, magnetic microparticles are used.

Microparticles can be useful for purification. Microparticles are generally spherical particles having a diameter in an inclusive range of from 0.01 μm to 10,000 μm, such as 0.05 μm to 1000 μm, such as 0.1 μm to 500 μm, on which a SBP (e.g., antibody, polynucleotide) can be bound or coated. Microparticles can be manufactured using materials such as glass, zirconium silicate, silica, gold, polystyrene, latex, and PMMA, and may have physical characteristics such as being magnetic or magnitizable, dyed, biodegradable and fluorescent. Microparticles can be coated with a binding agent, or can include reactive groups, such as amino, carboxyl, cyanuric groups, that allow covalent attachment with a binding partner (e.g., antibody, polynucleotide, avidin). In addition, microparticles can be obtained with bound SBPs, such as microparticles coated with streptavidin, antibodies to human IgG and IgM, and anti-biotin antibodies from a company such as Indicia Biotechnology (Oullins France); microparticles with binding groups: avidin, streptavidin, protein A, albumin, biotin, PEG, and collagen from, for example, Kisker Biotechnology (Steinfurt Germany). Other solid phases, such as microtiter plates can also be purchased with, or derivatized to have, covalently bound binding partners (e.g., microtiter plates with bound streptavidin or anti-IgG antibodies from BD Biosciences (Bedford Md.)).

The analyte can be bound to the immobilized binding agent by contacting the solution containing the analyte with the immobilized SBP under conditions in which the analyte is bound to the SBP. For example, microparticles can be added to a pre-sample solution. After attachment of the analyte to the microparticles an analyte-enriched fraction can be prepared by segregating the microparticles using centrifugation, magnetic separation, or filtration, with appropriate washing steps. Removal of unbound contaminants using other solid phases (e.g., microplate wells) can be accomplished by removing the unbound supernatant, and other methods.

b. Analyte is Associated with an Ionic Moiety

The analyte can be associated with an ionic moiety using any suitable method, such as those described in previous sections. As indicated above, the analyte can be associated with the ionic moiety before or after binding to the immobilized phase, and can be associated via the binding agent or directly. Thus, binding agents as used herein bind the analyte to an immobilized phase such as a microparticle or substrate. The ionic moiety can be associated with the binding agent associated with solid phase first. In this case, the association of the ionic moiety and the analyte is facilitated by binding to the solid phase. Alternatively, after binding the analyte to the binding agent associated with the solid phase, a specific binding partner (SBP) associated with an ionic moiety, which specifically binds to the analyte can be added to result in the analyte to be indirectly associated with an ionic moiety.

c. Analyte is Released from the Solid or Immobilized Phase

The bound analytes, with any associated ionic moieties, can be released from the microparticles (or other solid phase) using various methods. For example, an analyte can be displaced using specific agents to compete with the analyte for binding to the immobilized binding agent. Alternatively, the analyte can be eluted with non-specific reagents such as denaturing agents, e.g., urea, extreme pH, temperature, high ionic strength buffers, and the like to disrupt the binding. This can be done using, for example, changes in buffer, changes in the electric field, pH, or addition of an elution buffer.

Alternatively, the binding agents having the analyte and any associated ionic moieties bound thereto can be freed as a complex by incorporating a cleavable or breakable bond in the linkage between the binding agents and the solid phase (such as the microparticle or substrate). In addition, capture agents which are used to capture the analyte during analysis with the analysis assembly, such as the microfluidic assembly, e.g., on the microfluidic chip, can incorporate a cleavable or breakable linkage between the capturing agents and the capture surface on the assembly and/or microfluidic chip. While the binding agents and capture agents can be the same types of molecules, their roles are different. The capture agent is used when the analyte is captured during analysis, e.g., immobilized in the analysis element. In either case, the bonds include disulfide bridges, diols, restriction enzyme sequences, and bonds that can be dissociated by chemicals or enzymes. For example, the binding agent can be cleaved using proteases or nucleases, such as sequence-specific proteases or nucleases. If a linker containing a disulfide bridge is used to anchor the antibodies on the solid phase, the binding complex can be released by using reducing agents such as mercaptoethanol or dithiothreitol. A nucleic acid molecule can be cleaved with nucleases.

The following examples are provided for illustration and are not intended to limit the invention.

1. Nucleic Acid Analyte

In some embodiments, the analyte is a nucleic acid. Nucleic acid analytes are themselves poly-ionic so, while there may be circumstances in which attachment of an additional ionic moiety might be advantageous, it may not be necessary to attach an ionic moiety to mobilize these molecules with an electrical field. However, it may be advantageous to initially concentrate the analytes and/or remove contaminants before electrophoresis. This can be done, for example, using nucleic acid binding agents with complementary sequences conjugated to magnetic microparticles. The magnetic microparticles can be added to a sample containing the nucleic acid analyte in the large volume reservoir. After analyte molecules are bound to the microparticles, a magnetic field is applied to gather the microparticles at a specific spot in the device. Contaminants and interfering substances are then removed. Denaturation conditions such as low ionic strength, urea, betaine, high pH or temperature, or a combination of these factors, are used to disrupt the hybridization thereby separating the nucleic acid strands and freeing the bound analytes from the microparticles. Electrophoresis drives the analyte nucleic acids into a reservoir, e.g., a staging reservoir. Once concentrated in the reservoir, the nucleic acid analytes are propelled on the analysis assembly, such as a microfluidic assembly, and/or microfluidic device to specific capture sites having capture agents that are complementary to a first portion, e.g., the 5′ end, of the nucleic acid analytes. The nucleic acid analytes bind and can be detected using a labeled nucleic acid detection agent that is complementary to a second portion, e.g., the 3′ end, of the nucleic acid analytes.

2. Antigen Analyte

In another example, analytes that can act as antigens can be associated with a solid substrate using antibodies as binding agents. Antibodies with affinity to, for example, a protein analyte are conjugated to microparticles. The microparticles are gathered such as by magnetic means (if they are magnetic microparticles) or centrifugal field to a specific spot in the device, e.g., a large volume reservoir, and the supernatant containing any unbound molecules, contaminants or interfering substances is removed. The protein analytes are freed from the microparticle as a complex. This can be achieved by using cleavable linkers (e.g. as described herein). Appropriate proteases can also be used with optimized reaction conditions to achieve the desired release without undesirable degradation of the analyte. In addition, it is conceivable to engineer specific proteolytic sites in the anchoring components, either the linker or the antibody, for proteases with stringent cleavage site requirements. These proteases can then be used to cleave the binding complex. In some embodiments, an analyte in a blood sample is detected according to the methods of the invention using a pre-concentration step in which magnetic microparticles coated with binding agents with affinity to the analyte are added to the blood sample in a portion of a device e.g., a large volume reservoir.

### VII. Concentrative Electrophoresis of the Analyte

Some or all of the steps of associating the analyte with an ionic moiety (IM) (as well as prior sample processing steps) can be carried out, for example, in a first reservoir, e.g., a sample reservoir, such as a large volume reservoir. Alternatively, some or all of the steps can be carried out in one or more different vessels and the analyte (and any associated molecules) can be transferred to the device, e.g., a first reservoir, such as a large volume reservoir, portion of a device, by, for example, pipetting. As a result, the Analyte-IM Complex may be located in the first reservoir, e.g., a large volume reservoir.

The sample reservoir, e.g., large volume reservoir; and/or staging reservoir liquid capacity can vary significantly. Possible volumes of the sample reservoir and/or staging reservoir and/or sample are provided above. In addition, such volumes may range from 1 microliter to 100 milliliters. In some embodiments, the capacity (and sample volume) is 5 microliters or larger, such as 10 microliters or larger, such as 50 microliters or larger, 100 microliters or larger, and 1 milliliter or larger. In some embodiments, the capacity or sample volume, ranges from 1 microliter to 20 milliliters, inclusive. In some embodiments, the sample volume will be less than the reservoir capacity, though will sometimes be at least 25%, at least 50%, and/or at least 75% of the reservoir volume. In some embodiments, the sample has a volume ranging from 10 microliters to 100 milliliters, such as 25 microliters to 5 milliliters, such as 50 microliters to 5 milliliters or 100 microliters to 25 milliliters. The reservoir capacity may be 50 microliters, 100 microliters, 200 microliters, 300 microliters, 500 microliters, 800 microliters, 1 milliliter, 2 milliliters, 3 milliliters, 4 milliliters, 5 milliliters, 6 milliliters, 10 milliliters, 20 milliliters, 30 milliliters, 40 milliliters, 50 milliliters, 60 milliliters, 70 milliliters, 80 milliliters, and 90 milliliters. In general, a volume that can reasonably be expected to be concentratable by electrophoresis in a reasonable amount of time can be used. This may be determined by the amount of time it takes a charged analyte to move the longest distance in the sample volume.

Electrophoretic transfer of the charged analyte or the Analyte-IM Complex is accomplished by generating an electric field that extends from, for example, a reservoir, e.g., a sample reservoir, such as a large volume reservoir, via a connector to another portion of a device, such as a second reservoir of an electrophoresis assembly. By generating such an electric field, the charged analyte or the Analyte-IM Complex can be moved into a staging reservoir. The charged analyte or the Analyte-IM Complex can subsequently be moved, e.g., moved by pumping and/or electrophoresis, from the staging reservoir to the analysis element.

The electric field may be applied at a strength such that the analyte will be driven through the device, e.g., from the sample reservoir, e.g., large volume reservoir, to the staging reservoir, at a selected rate. The position of the electrodes and the electric field applied can be determined to facilitate the best performance for the device, for instance to achieve sensitive detection in short time. As an example, electrodes on within and/or on an upstream and/or downstream side of a sample reservoir can be placed at a point that offers most symmetry and distance in relation to the actuator and/or a portion thereof, e.g., the staging reservoir. Such an electrode placement may offer a relatively uniform electric field to drive all analytes in the reservoir. As used herein the term “downstream” generally refers to a direction, e.g., a direction of fluid flow, within a subject device which a sample and/or analyte travels in according to the methods described herein. In addition, the term “upstream” generally refers to a direction, e.g., a direction of fluid flow, within a subject device which is opposite from that of which a sample and/or analyte travels in according to the methods described herein. The placement of one or more electrodes in the analysis assembly, such as the microfluidic assembly, will depend on the intended operation of the device. The electrode(s) can be placed close to, such as within 10 cm of, the actuator and/or portion thereof, e.g., staging reservoir. It can be also placed close to, such as within 10 cm of, the analysis element. Electrodes can also be placed in both places such that electric fields can be applied to drive the analytes sequentially, out of the actuators first and then to the analysis element.

The time frame for the electrophoretic transfer can vary significantly depending on the scale of the device, the characteristics of the analyte, the compositions of the buffer(s), etc., for example, the electrophoretic transfer may be performed for a period of from 1 second to 1 year, such as from 1 second to 10 days, such as from 1 second to 5 days, such as from 1 second to 1 day, such as from 1 second to 1 hour, such as from 1 second to 20 minutes, such as from 1 second to 10 minutes, such as from 1 second to 5 minutes, such as from 1 second to 1 minute, such as from 1 second to 10 seconds.

The staging reservoir, in various embodiments, is constructed so as to be in fluidic communication with the analysis element when the actuator is in a particular configuration. The volume of the staging reservoir may be smaller than the volume of the sample reservoir, e.g., large volume reservoir, and may have any suitable volume as described herein. For example, in some embodiments, the staging reservoir has a volume of 1 microliter or less, including but not limited to, from 1 picoliter to 2 μl, 1 picoliter to 1 μl, and 10 picoliter to 0.5 μl., such as 1 microliter or less, sometimes 0.1 microliter or less, and sometimes 1 picoliter or less.

The connectors provided herein may be capillary tubes. To facilitate electrophoresis, a connector is filled with conductive media such as a low salt buffer so that an electrical field can be established. When the electric field is applied, the charged analyte or complex is transported and driven through a portion of a device into, for example, a staging reservoir to concentrate before further analysis and/or into an analysis assembly, such as a microfluidic assembly and/or an analysis element. The electrical potential of the electrodes are selected to achieve the desired rate of transport for the analyte.

The duration of electrophoresis depends on factors such as the concentration and amount of the analyte of interest in the sample, the electrophoretic conditions, e.g., current, voltage, ionic strength of buffers, etc., the volume of the initial sample, the charge of the analyte, the sensitivity of the method of detection, and the buffer that is used. Electrophoresis is carried out for a time sufficient to transport an amount of the analyte of interest. When sufficient analyte is transferred, electrophoresis can be discontinued.

In some embodiments, the electrophoresis is carried out until the concentration of analyte in the staging reservoir is at least 2-times the concentration in the large volume reservoir. Sometimes the difference in concentration between the two is at least 3-times, and sometimes at least 5-times, 10-times, 25-times, 100-times, 1000-times or greater.

More than one sample reservoir, e.g., large volume reservoir, and more than one staging reservoir may be connected to achieve the best configuration for the assay in order to transport the analyte with ease and efficiency. With very large samples, it can be more efficient to use multiple large volume reservoirs in series. In such an embodiment LVRs of decreasing volume are connected by connectors in sequence, with electrodes configured so that an analyte is transported serially from one LVR to the next and ultimately to the analysis assembly, such as the microfluidic assembly, via the actuator. This allows for voltages to be applied in such a way that analytes can be transferred with speed and minimizing the voltage required to transport analytes into the analysis assembly, such as the microfluidic assembly. A smaller voltage may be applied because, for example, it reduces any problems with electrolysis, such as heating and/or gassing in a microfluidic device.

In some embodiments, the analyte is transported from a staging reservoir to one or more intermediate reservoirs of the analysis assembly, such as the microfluidic assembly, prior to transport to the analysis element. This, for example, allows multiple assays, e.g., assays carried out in different solutions or with incompatible reagents, to be carried out. For example, the contents of a staging reservoir can be divided and one portion used for nucleic acid detection and the other for protein detection. Using intermediate reservoirs may also allow use of a larger volume of sample.

### VIII. Transport of Analyte, Charged Analyte, or Analyte-IM Complex to the Analysis Element

For detection and analysis of the analyte, the analyte (which may be associated with an ionic moiety and/or part of an analyte complex) is transported from a first portion of a device, e.g., a staging reservoir, to an analysis element of the device. In various embodiments, the analysis element includes multiple capture agents (which may be the same or different, and which may or may not be arranged in an ordered array) that bind the analyte or analyte-IM complex (and may bind the carrier molecule, ionic moiety, or other component of the complex).

Once the analyte and/or the complex including the analyte has been moved, e.g., moved by an actuation of an actuator, to the analysis assembly, such as the microfluidic assembly and/or the microfluidic chip, the analyte/complex can be transported to the analysis element or portion thereof, e.g., capture site, using a variety of elements, for example micropumps, capillary action components, and/or electrophoresis components. In some embodiments, electrophoresis is used. It will be appreciated that electrophoresis out of the staging reservoir across the array will require that an electric field be formed between a first portion of an analysis assembly, such as a microfluidic assembly, such as a portion upstream of a staging reservoir, and a second portion of an analysis assembly, such as a microfluidic assembly, such as an electrode in or near the analysis element which is downstream from the staging reservoir.

### IX. Analysis Element and Detection of Analyte

One or more analytes can be detected, quantified or analyzed in an analysis element or area as described herein. As discussed previously herein, such an analysis element may be a component of an analysis assembly of a device including an electrophoresis assembly or a separate device distinct from the device containing the electrophoretic assembly. In some embodiments, the analysis is done without immobilizing the analyte in the analysis element, e.g., performed using light scattering, flow cytometry, or fluorescent detection or other detection methods carried out in solution. For example, the analytes can be labeled with fluorophores and analyzed in the analysis element with an apparatus which may be used in flow cytometers to analyze fluorescent entities by exciting the fluorophores with a laser of appropriate wavelength and measuring the emitted fluorescent light. It will be appreciated that, consistent with the design of some microfluidic devices, the analysis element may be a channel or chamber of the analysis assembly, such as the microfluidic assembly.

Alternatively, or in addition, detection may involve binding of the analyte by an immobilized capture agent in the analysis element. A capture agent is a molecule that can capture an analyte or analyte complex by specifically binding the analyte or a member of the complex. Members of the complex that can be bound by the capture agent include the analyte, the carrier molecule, the carrier molecule/analyte complex, an ionic moiety, a second antibody (if used) and a label. Thus, the capture agent can be, for example, one or more antibodies, nucleic acids, receptors, and/or ligands.

The capture agent can be immobilized with respect to the analysis element and/or analysis assembly, such as the microfluidic assembly, at a capture site using a variety of methods. For example, one or more antibody capture agents can be attached via the Fc portion of the antibody. The capture agent can be attached to the capture site by covalent or non-covalent attachment, but, in some embodiments, the attachment is strong enough that the movement of liquid over the capture site will not detach the capture agents. Details regarding various reagents and reaction conditions used may be found, for example, in: “Chemical Modification of Proteins” by Gary E. Means and Robert E. Feeney, “Bioconjugate Techniques” by Greg T. Hermanson and “Chemical Reagents for Protein Modification” by Roger L. Lundblad. Various methods of attachment of capture agents to capture sites on solid phase substrates can be found, for example, in U.S. Pat. Nos. 5,629,213, 5,688,642, 5,585,275, and International Patent Application WO9745730, the disclosures each of which are hereby incorporated by reference in their entireties and for all purposes.

Once attached at the capture site, the capture agents can capture the analyte or a member of the analyte complex as it is moved over the capture site. The capture agent and analyte or analyte complex may bind by a noncovalent interaction, but covalent binding may also be used. For nucleic acid analytes or nucleic acid ionic moieties, capture agents can be, for example, complementary nucleic acids. For protein analytes, capture agents can include antibodies, ligands, lectins and receptors specific for the protein analyte. Carbohydrate and lipid analytes can be captured using antibody capture agents. Antibody carrier molecules can be captured using antibodies, antigens or other molecules that specifically bind antibodies. For example, if the carrier molecule is a human antibody, the capture agent can be a goat anti-human antibody.

The conditions for capture can be manipulated to allow specific capture of a targeted member of the analyte complex. Conditions such as the buffer, the rate of movement over the capture site, the temperature and the capture time can be chosen to allow binding of only a specific analyte or to also bind to variants such as nucleic acids with single base changes or variant proteins. Alternatively, the capture agent can be chosen to bind only a specific analyte or to variants.

The capture agents can be directed to a specific location or capture site on an analysis assembly, such as a microfluidic assembly, by a variety of methods. For example, the capture agents can be directed and associated to a specific capture site using electrophoresis or pumping devices, such as pumping devices which are configured to pump a liquid and/or gas, such as water or air, or one or more other propulsion aspects.

In some embodiments, an analyte is detected based on a signal from a detectable label. A label refers to an atom, such as a radionuclide, molecule, such as fluorescein, or complex, that is or can be used to detect (e.g., due to a physical or chemical property), the presence of a molecule or to enable binding of another molecule to which it is covalently bound or otherwise associated. The term “label” also refers to covalently bound or otherwise associated molecules (e.g., a biomolecule such as an enzyme) that act on a substrate to produce a detectable physical signal, molecule or complex. Detectable labels suitable for use in association with the subject methods and devices include any composition detectable by spectroscopic, photochemical, biochemical, immunochemical, electrical, optical, chemical or physical means and the like. A detectable label, when used, can be added to any component of the final complex which may be bound to the capture agent. For example, the label can be bound to a carrier molecule, an analyte, an ionic moiety, or a binding agent. It will also be appreciated that multiple analytes can be detected in the same sample.

### X. Specificity of the Assay

The specificity of the assay can be provided at any one or more of the steps in the methods described herein. For example, one or more steps can involve binding to general classes of molecules and one or more steps can require specific binding to only the analyte of interest. When specificity is desired, a specific ionically-modified antibody can be used, a specific carrier molecule can be used, a specific binding agent can be used, a specific detection molecule can be used, and/or a specific capture agent can be used on the analysis assembly, such as the microfluidic assembly, and/or microfluidic device. Thus, for example, initially the analyte in the sample can be concentrated using microparticles coated with specific binding molecules. Alternatively, the microparticles can be coated with binding molecules that bind a general class of molecules that includes the analyte. Classes of general molecules includes: phosphorylated proteins, glycoproteins, nucleic acids, antibodies, lipids, sugars, shared receptor domains, shared motifs, conservative nucleic acid or amino acid motifs, shared carriers, and shared epitopes.

### XI. Antibodies Used in the Assay

It will be appreciated that antibodies may play several roles in the method of the subject disclosure. For example, an antibody can also be the analyte, as described in the example below. The term “antibody” refers to immunoglobulins including two heavy and two light chains, and antigen binding fragments thereof (including, e.g., Fab, Fab′ F(ab′)2, Fabc, and Fv). Fragments may be produced by recombinant DNA techniques, or by enzymatic or chemical separation of intact immunoglobulins. The term “antibody” also includes one or more immunoglobulin chains or fragments that may be chemically conjugated to, or expressed as, fusion proteins with other proteins, single chain antibodies, and bispecific antibodies. See, e.g., Harlow & Lane, Antibodies, A Laboratory Manual, Cold Spring Harbor Press, New York (1988); Current Protocols in Immunology (J. E. Coligan et al., eds., 1999, including supplements through 2005); Songsivilai & Lachmann, Clin. Exp. Immunol. 79:315-321 (1990); Kostelny et al., J. Immunol. 148, 1547-1553 (1992). Antibodies can be monoclonal or polyclonal. However, in some embodiments, monoclonal antibodies are used to ensure binding to a specific epitope on the protein.

In some embodiments, two or more antibodies are bound to the analyte (or other analyte-IM complex component) concurrently at some point in the assay. Under such conditions, the antibodies either bind different epitopes on the analyte, a second antibody binds to a first antibody, or a second antibody binds to the antibody/analyte complex. In other embodiments, a first antibody can be removed prior to addition of a second antibody.

Depending on the function they serve, antibodies can be ionically-labeled and/or be modified to include a detectable label for subsequent detection. As used herein, a “detectable label” has the ordinary meaning in the art and refers to an atom, such as a radionuclide, molecule, such as fluorescein, or complex, that is or can be used to detect (e.g., due to a physical or chemical property), the presence of a molecule or to enable binding of another molecule to which it is covalently bound or otherwise associated. The term “label” also refers to covalently bound or otherwise associated molecules (e.g., a biomolecule such as an enzyme) that act on a substrate to produce a detectable atom, molecule or complex. Detectable labels suitable for use in the present invention include any composition detectable by spectroscopic, photochemical, biochemical, immunochemical, electrical, optical, chemical or physical means and the like.

### XII. Apparatus and System

The subject disclosure provides an apparatus and system for carrying out the methods described herein. In some embodiments, a suitable apparatus includes one or more devices as described herein, e.g., a device including an electrophoresis assembly and an analysis assembly, such as a microfluidic assembly, and a staging reservoir in operable, such as fluidic, communication with an analysis element that includes capture agents that bind (i) the analyte, (ii) an antibody or (iii) a nucleic acid (e.g., carrier molecules or elements of the analyte-IM complex). In accordance with the subject disclosure, the subject devices or portions thereof, e.g., an electrophoresis assembly, are configured to move an analyte via electrophoresis from a first portion to a second portion of a device and to thereby concentrate the analyte at a particular location. For example, an analyte may be electrophoretically transported within a device, e.g., transported directly to a staging reservoir from a sample reservoir or via a connector to a staging reservoir. As indicated above, electrophoresis refers to the movement of charged molecules or particles in solution in response to an electric field. The mobility of an analyte is based on (1) a net charge of the analyte molecule itself and/or (2) a net charge of an ionic moiety associated with the analyte, as described below. A net charge is the combination ionic charge that a molecule has, and it can be positive, negative, or neutral. As noted above, the analyte may be electrophoresed through an aqueous buffer solution with or without the use of size exclusion gels, a viscous medium, filters, biological sieves or the like.

In some embodiments, such as the embodiment of the device shown in FIG. 1, the device is configured such that an analyte, including one or more particles associated with, e.g., bound to, an analyte, may be electrophoresed from sample reservoir 102 into the actuator 105 or a portion thereof, e.g., a staging reservoir, using the electrophoresis assembly 101. The actuator 105 is configured to be actuated, e.g., manually or automatically actuated, such as actuated by rotation, such as actuated by rotation within a vertical plane, e.g., a plane perpendicular to a ground surface, or a horizontal plane, to disconnect the staging reservoir from the electrophoresis assembly 101 or a portion thereof, such as a sample reservoir, and/or operably connect the staging reservoir with a microfluidic assembly 103 and/or an analysis element 104. In some embodiments, actuation includes rotating an actuator or a portion thereof along an arc length which defines a vertical or horizontal plane. The microfluidic assembly 103 is configured to thereafter move the analyte out of the staging reservoir of the actuator 105 to an analysis element 104. Additional actuator types and configurations are discussed previously herein.

Additionally, the elements shown in FIG. 1, e.g., sample reservoir and/or connector 112, etc., may be filled with fluid, such as one or more suitable buffers that serve the purpose of operably connecting the electrodes 107 and/or 108 to form an electrical field. Forming such an electric field may allow a charged analyte to be electrophoresed, for example, from a sample reservoir 102 to a staging reservoir of actuator 105.

In some embodiments, as described previously herein, a system according to the present disclosure may include an electrophoresis assembly in a first device and the analysis element in a second, distinct device. As noted above, the analysis element may include a region of a microfluidic channel where the capture agents are immobilized on at least a surface of the channel. The device may include, as appropriate, a propelling element, for example, upstream of the staging reservoir within the analysis assembly, such as the microfluidic assembly. The propelling element can be configured to drive one or more molecule from the staging reservoir to the analysis element. Alternatively, as discussed previously herein a concentrated analyte-containing sample or analyte contained therein may be transferred, such as transferred indirectly, to an analysis element, such as an analysis element of a separate device manually or using one or more robotic components and/or semi-automated methods, such as methods of which, at least a part, or only a part, involve a computer-controlled transfer. In such embodiments, a suitable system may include suitable transfer elements and/or devices, such as one or more robotic components or devices, and may also include an applicable computer processor and user interface.

In some embodiments, a suitable device includes multiple units of staging reservoirs and/or analysis elements, as described above. Each combination of staging reservoirs and analysis elements can be referred to as an “operational unit.”

In some embodiments, at least one analysis element of device suitable device includes capture agents that bind molecules from more than one pathogenic organism. In an embodiment, at least one, and optionally all of the pathogenic organisms, are viruses. In an embodiment, at least one, and optionally all of the pathogenic organisms, are bacteria. In some embodiments, molecules from pathogenic organisms are nucleic acids, proteins, or toxins.

In some embodiments, the subject disclosure includes a system that includes an analysis element and/or an analysis assembly, such as a microfluidic assembly, and further includes a sample reservoir, e.g., large volume reservoir, and/or connector and/or source of electric current and/or a computer-implemented control system that can be used to activate and/or switch polarity of the electrodes as needed for concentration, transport and/or analysis. In some embodiments, the system uses electrophoresis to concentrate a charged analyte and/or to introduce or apply the sample to an analysis element and/or an analysis assembly, such as a microfluidic assembly. Electrophoresis is used to move and concentrate a charged analyte from a sample reservoir, such as a large volume reservoir, into an actuator, or a portion thereof, such as a staging reservoir, and then optionally into an analysis assembly, such as a microfluidic assembly. In some embodiments, the analysis assembly, such as the microfluidic assembly also includes an effluent well for collection of any effluent left over from the sample analysis, e.g., an effluent well downstream of the analysis element.

Electrodes of the subject devices can be connected to portions of the device, e.g., reservoirs and/or analysis assembly portions, such as microfluidic assembly portions using a method which will produce an appropriate electric field. The electrodes may be operably coupled to the reservoirs of the device. For example, electrodes may be positioned in or near a reservoir so that application of a voltage or potential generates an electric field through which charged molecules are transported. In some embodiments, the electrode is embedded in the material, e.g., contained in the substrate, of a subject device or a portion thereof, such as a reservoir. Further, when more than two electrodes are present, a control system, such as a computer-implemented control system, can be used to switch polarity of the electrodes as needed for concentration, transport and/or analysis. The control system can be configured to be responsive to one or more detector that measures the presence and/or position of analyte in the analysis element.

In some embodiments, the system includes a multiplicity of operational units each in fluidic communication with a different sample reservoir, e.g., large volume reservoir. In some embodiments, the system includes a multiplicity of operational units, each with an actuator and staging reservoir thereof, an associated electrode, and a control system that applies a charge of the same magnitude and duration to each of the electrodes.

According to the subject embodiments, various types of instrumentation can be used for applying voltage, controlling fluid transport, flow rate and direction within the device. Detection instrumentation may also be applied for detecting or sensing the analyte of interest, such instrumentation may include processors, e.g. computers for instructing the controlling instrumentation, receiving data from the detectors, analyzing, storing and interpreting the data, and/or providing the data and interpretations in a readily accessible reporting format, such as on a display.

### XIII. Applications

Analyte detection systems, such as systems including electrophoretic and/or microfluidic components, have significant potential for use in a clinical laboratory setting. However, the efficiency of such devices is sometimes limited because they have capacity for only very small sample volumes. In other words, some systems cannot accept samples larger than a particular volume. When substances to be analyzed are found in a sample at a very low concentration, sensitivity can be limited. In other words, some systems cannot detect analytes below a certain concentration. One way to overcome this limitation is by using an analyte amplification step to increase the analyte concentration either before or after introduction of the sample to an analysis assembly, such as a microfluidic assembly and/or a microfluidic device. For example, very small amounts of nucleic acids can be amplified using methods such as PCR. However, not all analytes can be amplified and, even when possible, amplification may require additional reagents and increase the complexity of analysis. Methods that allow analytes from large volume samples to be assayed, for example in a microfluidic format, and/or allow analyses without an amplification step, such as those disclosed herein, are valuable for clinical and other laboratory assays. The subject matter of present disclosure meets this and many other needs.

In short, the disclosed subject matter significantly improves the limit of detection (“LOD”), which is the lowest analyte concentration in a sample that can be detected, by a detection device by enabling efficient capture of analyte molecules in a sample, such as a large volume sample, in a considerably smaller detection area and volume than that of conventional methods. For example, the disclosed subject matter includes a method and apparatus to concentrate a diluted analyte sample, and to reduce its volume down to a volume that approximates the volume of a detection chamber and/or analysis element. By concentrating the analyte molecules available in a large volume down to a volume approximating that of the detection chamber, the analyte can be detected with systems that have a limitation in sample volume and/or analyte concentration detection capability. Also, by taking such action, it is possible to expose a majority of analyte molecules from the original sample for an extended period of time to a capture surface, resulting in efficient analyte capture on the detection surface and thus greatly enhancing the LOD of the detection technology.

### XIV. Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-80 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

1. A device including:

an electrophoresis assembly including a sample reservoir; and

an actuator including a staging reservoir and actuable between a first configuration and a second configuration, wherein the staging reservoir is fluidically connected with the sample reservoir when the actuator is in the first configuration and not fluidically connected to the sample reservoir when the actuator is in the second configuration.

2. The device according to 1, wherein the electrophoresis assembly is configured to move the analyte via electrophoresis from the sample reservoir to the staging reservoir.
3. The device according to 2, wherein the electrophoresis assembly includes a first electrode and a second electrode, wherein the electrodes are configured so that an electric field may be generated between the electrodes, and wherein generating an electric field between the electrodes moves an analyte via electrophoresis from the sample reservoir to the staging reservoir.
4. The device according to any one of 1-3, wherein the electrophoresis assembly includes a first buffer reservoir operably connected to the sample reservoir and a second buffer reservoir.
5. The device according to 4, wherein the first buffer reservoir is operably connected to the second buffer reservoir via the staging reservoir when the actuator is in the first configuration.
6. The device according to 5, wherein the staging reservoir is operably connected to a fluidic connector when the actuator is in the second configuration.
7. The device according to 6, wherein the fluidic connector is operably connected to an analysis element.
8. The device according to 5, wherein the first buffer reservoir is not operably connected to the second buffer reservoir when the actuator is in the second configuration.
9. The device according to any one of 1-8, wherein the electrophoresis assembly includes a filtering element configured to inhibit movement of the analyte through the filtering element.
10. The device according to 9, wherein the filtering element is positioned downstream of the staging reservoir and configured to concentrate the analyte in or near the staging reservoir.
11. The device according to any one of 1-10, wherein the sample has a volume of 1 μL or greater.
12. The device according to any one of 1-11, wherein the sample reservoir is a large volume reservoir.
13. The device according to 12, wherein the large volume reservoir is configured to receive a sample having a volume of 1 μL or greater.
14. The device according to 12, wherein the staging reservoir is operably connected to the large volume reservoir via a connector when the actuator is in the first configuration.
15. The device according to 12, wherein the staging reservoir is not operably connected to the large volume reservoir via a connector when the actuator is in the second configuration.
16. The device according to any one of 1-15, wherein the actuator includes a valve configured such that the valve can be rotated or slid between the first configuration and the second configuration.
17. The device of 16, wherein the valve is configured such that a 90° rotation moves the actuator from the first configuration to the second configuration or from the second configuration to the first configuration.
18. The device according to any one of 1-17, wherein the device includes a housing, and wherein at least a portion of the sample reservoir is defined by the housing.
19. The device according to any one of 1-18, wherein the device includes a housing, and wherein at least a portion of the actuator including the staging reservoir is positioned within the housing.
20. The device according to any one of 1-15, wherein the actuator is a sliding actuator.
21. The device according to any one of 1-15, wherein the actuator is a disk actuator.
22. A device for detection of an analyte including:

an electrophoresis assembly including a sample reservoir;

an analysis assembly including an analysis element; and

an actuator including a staging reservoir and actuable between a first configuration and a second configuration, wherein the staging reservoir is operably connected with the sample reservoir when the actuator is in the first configuration and is operably connected with the analysis element when the actuator is in the second configuration.

23. The device according to 22, wherein the electrophoresis assembly is configured to move the analyte via electrophoresis from the sample reservoir to the staging reservoir.
24. The device according to 23, wherein the electrophoresis assembly includes a first electrode and a second electrode, wherein the electrodes are configured so that an electric field may be generated between the electrodes, and wherein generating an electric field between the electrodes moves an analyte via electrophoresis from the sample reservoir to the staging reservoir.
25. The device according to any one of 22-24, wherein the electrophoresis assembly includes a first buffer reservoir operably connected to the sample reservoir and a second buffer reservoir.
26. The device according to 25, wherein the first buffer reservoir is operably connected to the second buffer reservoir via the staging reservoir when the actuator is in the first configuration.
27. The device according to 26, wherein the staging reservoir is operably connected to a fluidic connector when the actuator is in the second configuration.
28. The device according to 27, wherein the fluidic connector is operably connected to an analysis element.
29. The device according to 26, wherein the first buffer reservoir is not operably connected to the second buffer reservoir when the actuator is in the second configuration.
30. The device according to any one of 22-29, wherein the electrophoresis assembly includes a filtering element configured to inhibit movement of the analyte through the filtering element.
31. The device according to 30, wherein the filtering element is positioned downstream of the staging reservoir and configured to concentrate the analyte in the staging reservoir.
32. The device according to any one of 22-31, wherein the analysis assembly includes a propulsion assembly configured to propel the analyte from the staging reservoir to the analysis element.
33. The device according to any one of 22-32, wherein the analysis assembly includes a first electrode and a second electrode, wherein the electrodes are configured such that an electric field may be generated between the electrodes, and wherein generating an electric field between the electrodes moves an analyte via electrophoresis from the staging reservoir to the analysis element.
34. The device according to any one of 22-33, wherein the sample has a volume of 1 μL or greater.
35. The device according to any one of 22-34, wherein the sample reservoir is a large volume reservoir.
36. The device according to 35, wherein the large volume reservoir is configured to receive a sample having a volume of 1 μL or greater.
37. The device according to 35, wherein the staging reservoir is operably connected to the large volume reservoir via a connector when the actuator is in the first configuration.
38. The device according to any one of 35-37, wherein the actuator includes a valve configured such that the valve can be rotated or slid between the first configuration and the second configuration.
39. The device according to 38, wherein the valve is configured such that a 90° rotation moves the actuator from the first configuration to the second configuration or from the second configuration to the first configuration.
40. The device according to any one of 22-38, wherein the actuator is a sliding actuator.
41. The device according to any one of 22-38, wherein the actuator is a disk actuator.
42. The device according to any one of 22-41, wherein the device includes a housing, and wherein at least a portion of the sample reservoir is defined by the housing.
43. The device according to any one of 22-42, wherein the device includes a housing, and wherein at least a portion of the actuator including the staging reservoir is positioned within the housing.
44. The device according to any one of 22-43, wherein the analysis assembly is a microfluidic assembly.
45. A method for introducing an analyte in a sample to an analysis element, the method including:

electrophoresing the sample including the analyte into an actuator including a staging reservoir;

actuating the actuator to operably connect the staging reservoir to the analysis element; and

propelling the sample into the analysis element.

46. The method according to 45, wherein the sample is electrophoresed into the actuator using an electrophoresis assembly.
47. The method according to 46, wherein electrophoresing the sample including the analyte into the actuator includes generating an electric field between a first electrode at a first end of the electrophoresis assembly and a second electrode at a second end of the electrophoresis assembly.
48. The method according to 46, wherein the electrophoresis assembly includes a sample reservoir and wherein electrophoresing the sample into the actuator includes electrophoresing the analyte from the sample reservoir to the staging reservoir.
49. The method according to 48, wherein the sample reservoir is a large volume reservoir.
50. The method according to 49, wherein the large volume reservoir is configured to receive a sample having a volume of 1 μL or greater.
51. The method according to any one of 45-50, wherein the sample has a volume of 1 μL or greater.
52. The method according to 49, wherein the staging reservoir is operably connected to the large volume reservoir by a connector, and wherein the sample is electrophoresed into the actuator via the connector.
53. The method according to any one of 45-52, wherein electrophoresing the sample into the actuator includes concentrating the analyte in the staging reservoir.
54. The method according to any one of 46-53, wherein the electrophoresis assembly includes a filtering element and electrophoresing the sample includes generating an electric field across the filtering element.
55. The method according to any one of 45-54, wherein the sample is propelled into the analysis element via an analysis assembly, and wherein propelling the sample into the analysis element includes generating an electric field between a first electrode at a first end of the analysis assembly and a second electrode at a second end of the analysis assembly.
56. The method according to any one of 45-55, wherein the sample is propelled into the analysis element via an analysis assembly, and wherein propelling the sample into the analysis element of the analysis assembly includes applying fluidic pressure to the sample.
57. The method according to any one of 45-56, wherein the actuator includes a valve and actuating the actuator includes rotating the valve from the first configuration to the second configuration.
58. The method according to 57, wherein actuating the actuator includes rotating the valve 90°.
59. The method according to 55 or 56, wherein the analysis assembly is a microfluidic assembly.
60. The method according to any one of 45-56, wherein the actuator is a sliding actuator and actuating the actuator includes sliding the actuator from the first configuration to the second configuration.
61. The method according to any one of 45-56, wherein the actuator is a disk actuator.
62. A system including:

a first device including:
an electrophoresis assembly including a sample reservoir; and
an actuator including a staging reservoir and actuable between a first configuration and a second configuration, wherein the staging reservoir is operably connected with the sample reservoir when the actuator is in the first configuration and wherein the staging reservoir is accessible for transfer of a sample therefrom when the actuator is in the second configuration; and

a second device including:
an analysis element configured for analyzing the sample.
   63. The system according to 62, wherein the second device is unattached to the first device.
   64. The system according to 62, wherein the second device is removably coupled to the first device.
   65. A method for introducing an analyte in a sample to an analysis element, the method including:

electrophoresing the sample including the analyte from a sample reservoir into an actuator including a staging reservoir, wherein the actuator is actuable between a first configuration and a second configuration, and wherein the staging reservoir is fluidically connected with the sample reservoir when the actuator is in the first configuration and not fluidically connected to the sample reservoir when the actuator is in the second configuration;

actuating the actuator, after the electrophoresing, to the second configuration; and

transferring the analyte to an analysis element when the actuator is in the second configuration.

66. The method according to 65, wherein the sample is electrophoresed into the actuator using an electrophoresis assembly.
67. The method according to 66, wherein electrophoresing the sample including the analyte into the actuator includes generating an electric field between a first electrode at a first end of the electrophoresis assembly and a second electrode at a second end of the electrophoresis assembly.
68. The method according to 65, wherein the electrophoresis assembly includes a sample reservoir and wherein electrophoresing the sample into the actuator includes electrophoresing the analyte from the sample reservoir to the staging reservoir.
69. The method according to 68, wherein the sample reservoir is a large volume reservoir.
70. The method according to 69, wherein the large volume reservoir is configured to receive a sample having a volume of 1 μL or greater.
71. The method according to any one of 65-70, wherein the sample has a volume of 1 μL or greater.
72. The method according to 70, wherein the staging reservoir is operably connected to the large volume reservoir by a connector, and wherein the sample is electrophoresed into the actuator via the connector.
73. The method according to any one of 65-72, wherein electrophoresing the sample into the actuator includes concentrating the analyte in the staging reservoir.
74. The method according to any one of 65-73, wherein the electrophoresis assembly includes a filtering element and electrophoresing the sample includes generating an electric field across the filtering element.
75. The method according to any one of 65-74, wherein the sample is transferred into the analysis element automatically.
76. The method according to any one of 65-74, wherein the sample is transferred into the analysis element manually.
77. The method according to any one of 65-76, wherein the actuator includes a valve and actuating the actuator includes rotating the valve from the first configuration to the second configuration.
78. The method according to 77, wherein actuating the actuator includes rotating the valve 90°.
79. The method of any one of 65-76, wherein the actuator is a sliding actuator and actuating the actuator includes sliding the actuator from the first configuration to the second configuration.
80. The method of any one of 65-77, wherein the actuator is a disk actuator.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### I. Example 1

Samples containing varying concentrations of target analyte were processed in the device described herein and subsequently analyzed in a microfluidic detection device as follows: A 50 μl sample containing the target analyte was loaded into the sample reservoir of a device as described in FIGS. 3 and 4. The remaining volume of the sample reservoir was filled with a buffer including: 10 mM Tris, 25 mM MES, pH6.0, 0.01% Tween20, 0.1 mM ethylenediaminetetraacetic acid (EDTA). The lower buffer reservoir and fluidic path connecting the sample reservoir and the lower reservoir were filled with a buffer including: 0.5M Tris, 90 mM phosphoric acid, pH 8.7, 0.01% Tween20, 1 mM EDTA. A 10,000 dalton molecular weight cut-off (MWCO) regenerated cellulose membrane was positioned below the staging reservoir. In this example, the target analyte consisted of a partially double stranded complex between a 100 nt and a 75 nt oligonucleotide with a streptavidin-HRP conjugate bound to a biotin on one of the nucleic acid strands. Like all nucleic acids, the complex has a highly negative charge due to its phosphate backbone. An electrical field of 100 V was applied between the sample reservoir and the lower buffer reservoir, with the positively charged electrode in the lower reservoir, for 25 minutes. The polarity was then reversed for 15 seconds. After the electrophoresis process is completed and the actuator valve is rotated by 90 degrees, the 0.4 μl processed sample in the staging reservoir can be transferred to any suitable detection device for analysis. In this case, the 0.4 μl processed sample in the staging reservoir was pumped to the detection zone of a microfluidic device that has a 0.2 μl detection chamber for analysis.

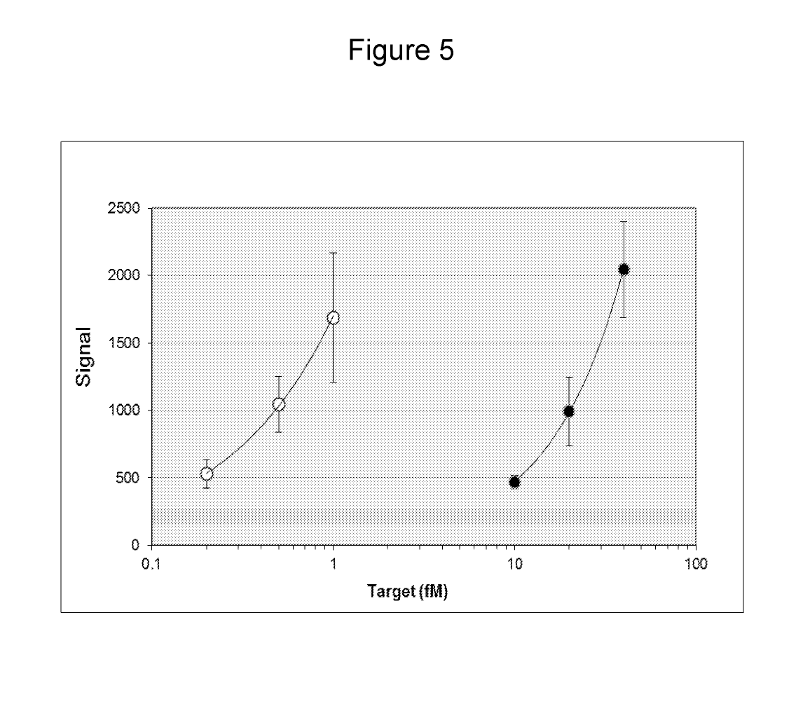

FIG. 5 compares the lowest analyte concentration measurable by direct measurement of a non-processed 0.4 μl sample in the microfluidic detection device (closed circles), with the lowest measurable concentration in a 50 μl sample which is processed down to 0.4 μl as described here and subsequently analyzed. The lowest analyte concentration measurable by direct analysis of a 0.4 μl sample was 10 fM, while the lowest concentration measurable in processed 50 μl sample was 0.2 fM. The shaded area corresponds to the background signal in this test.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A device comprising:
an electrophoresis assembly comprising a sample reservoir; and
an actuator comprising a staging reservoir and actuable between a first configuration and a second configuration, wherein the staging reservoir is fluidically connected with the sample reservoir when the actuator is in the first configuration and not fluidically connected to the sample reservoir when the actuator is in the second configuration.

2. The device according to claim 1, wherein the electrophoresis assembly is configured to move the analyte via electrophoresis from the sample reservoir to the staging reservoir.

3. The device according to claim 2, wherein the electrophoresis assembly comprises a first electrode and a second electrode, wherein the electrodes are configured so that an electric field may be generated between the electrodes, and wherein generating an electric field between the electrodes moves an analyte via electrophoresis from the sample reservoir to the staging reservoir.

4. The device according to claim 1, wherein the electrophoresis assembly comprises a first buffer reservoir operably connected to the sample reservoir and a second buffer reservoir.

5. The device according to claim 4, wherein the first buffer reservoir is operably connected to the second buffer reservoir via the staging reservoir when the actuator is in the first configuration.

6. The device according to claim 5, wherein the staging reservoir is operably connected to a fluidic connector when the actuator is in the second configuration.

7. The device according to claim 6, wherein the fluidic connector is operably connected to an analysis element.

8. The device according to claim 5, wherein the first buffer reservoir is not operably connected to the second buffer reservoir when the actuator is in the second configuration.

9. The device according to claim 1, wherein the electrophoresis assembly comprises a filtering element configured to inhibit movement of the analyte through the filtering element.

10. The device according to claim 9, wherein the filtering element is positioned downstream of the staging reservoir and configured to concentrate the analyte in or near the staging reservoir.

11. The device according to claim 1, wherein the sample has a volume of 1 μL or greater.

12. The device according to claim 1, wherein the sample reservoir is a large volume reservoir.

13.-15. (canceled)

16. The device according to claim 1, wherein the actuator comprises a valve configured such that the valve can be rotated or slid between the first configuration and the second configuration.

17. The device of claim 16, wherein the valve is configured such that a 90° rotation moves the actuator from the first configuration to the second configuration or from the second configuration to the first configuration.

18. The device according to claim 1, wherein the device comprises a housing, and wherein at least a portion of the sample reservoir is defined by the housing.

19. The device according to claim 1, wherein the device comprises a housing, and wherein at least a portion of the actuator including the staging reservoir is positioned within the housing.

20. The device according to any claim 1, wherein the actuator is a sliding actuator.

21. The device according to claim 1, wherein the actuator is a disk actuator.

22. A device for detection of an analyte comprising:
an electrophoresis assembly comprising a sample reservoir;
an analysis assembly comprising an analysis element; and
an actuator comprising a staging reservoir and actuable between a first configuration and a second configuration, wherein the staging reservoir is operably connected with the sample reservoir when the actuator is in the first configuration and is operably connected with the analysis element when the actuator is in the second configuration.

23.-44. (canceled)

45. A method for introducing an analyte in a sample to an analysis element, the method comprising:
electrophoresing the sample comprising the analyte into an actuator comprising a staging reservoir;
actuating the actuator to operably connect the staging reservoir to the analysis element; and
propelling the sample into the analysis element.

46.-64. (canceled)

65. A method for introducing an analyte in a sample to an analysis element, the method comprising:
electrophoresing the sample comprising the analyte from a sample reservoir into an actuator comprising a staging reservoir, wherein the actuator is actuable between a first configuration and a second configuration, and wherein the staging reservoir is fluidically connected with the sample reservoir when the actuator is in the first configuration and not fluidically connected to the sample reservoir when the actuator is in the second configuration;
actuating the actuator, after the electrophoresing, to the second configuration; and
transferring the analyte to an analysis element when the actuator is in the second configuration.

66.-80. (canceled)
